(19) United States
(12) Patent Application Publication
(10) Pub. No.: US 2020/0347140 A1
(43) Pub. Date: Nov. 5, 2020

(54) COMPOSITIONS AND METHODS FOR TREATING CANCER WITH ANTI-EGFR ANTIBODIES

(71) Applicant: SYMPHOGEN A/S, Ballerup (DK)

(72) Inventors: Michael Kragh, Copenhagen N (DK); Thomas Tuxen POULSEN, Dyssegaard (DK); Cliff DING, Princeton, NJ (US); Ivan David Horak, West Orange, NJ (US)

(73) Assignee: SYMPHOGEN A/S, Ballerup (DK)

(21) Appl. No.: 16/642,589

(22) PCT Filed: Aug. 29, 2018
(86) PCT No.: PCT/EP2018/073243

§ 371 Date: Feb. 27, 2020

Related U.S. Application Data
(60) Provisional application No. 62/552,125, filed on Aug. 30, 2017.

(51) Int. Cl. C07K 16/28; C07K 16/30; C12Q 1/6886; A61P 35/00

(52) U.S. Cl. CPC C07K 16/2863; C07K 16/3046; A61K 2039/507; A61P 35/00; C12Q 1/6886

(57) ABSTRACT

The present invention provides methods and uses of anti-EGFR antibody compositions for treatment of cancers that are negative for certain mutations in RAS, BRAF, and the EGFR extracellular domain and are resistant to other anti-EGFR therapies.

### CROSS-REFERENCE TO RELATED APPLICATION

This application claims priority from U.S. Provisional Patent Application 62/552,125, filed Aug. 30, 2017. The disclosure of that application is incorporated by reference herein in its entirety.

### SEQUENCE LISTING

The instant application contains a Sequence Listing which has been submitted electronically in ASCII format and is hereby incorporated by reference in its entirety. The electronic copy of the Sequence Listing, created on Aug. 20, 2018, is named 022675_WO059_SL.txt and is 50,335 bytes in size.

### BACKGROUND OF THE INVENTION

Epidermal Growth Factor Receptor (EGFR) plays an important role in cellular proliferation as well as apoptosis, angiogenesis, and metastatic spread, processes that are crucial to progression of cancers such as metastatic colorectal cancer (mCRC). Monoclonal antibodies (mAbs) directed to the ligand-binding domain of EGFR can block interaction with EGFR ligands and thus inhibit the resultant intracellular signaling pathway. A variety of approaches aimed at improving the inherent efficacy of individual anti-EGFR mAbs have been described, including enhancement of potential immune functions such as ADCC. However, so far none has demonstrated a clinical advantage. Combination of an anti-EGFR antibody with different chemotherapy regimens in first, second, and subsequent lines of therapy has led to clinically significant improvements in outcomes in mCRC patients with tumors having wildtype RAS (i.e., WT RAS tumors). However, a significant proportion of patients with mCRC treated with the currently available anti-EGFR mAbs alone or in combination with chemotherapy have intrinsic resistance to this therapeutic approach. Furthermore, patients who initially respond often develop acquired resistance over time (Misale et al., Nature 486:532-536 (2012); Diaz et al., Nature 486:537-540 (2012); Montagut et al., Nat. Med. 18:221-223 (2012); and Siravegna et al., Nat. Med. 21:795-801 (2015)).

Panitumumab and cetuximab are two anti-EGFR mAbs approved for treatment of WT RAS mCRC as single agents or in combination with irinotecan in chemotherapy-refractory patients, as well as in combination with FOLFOX (folinic acid, fluorouracil and oxaliplatin) or FOLFIRI (folinic acid, fluorouracil and irinotecan) in earlier lines of therapy (Van Cutsem et al., J. Clin. Oncol. 29:2011-9 (2011); Bokemeyer et al., Ann. Oncol. Off. J. Eur. Soc. Med. Oncol. 22:1535-46 (2011); and Peeters et al., Clin. Cancer Res. 21:5469-79 (2015)). However, in this setting, the response is transient due to the occurrence of acquired (secondary) resistance (Misale et al., supra; Diaz et al., supra; Montagut et al., supra; Misale et al., supra; and Arena et al., Clin. Cancer Res. 21:2157-66 (2015)).

Over the last two decades, the rapid development of technologies to evaluate potential genetic alterations in patients' tumors at baseline and during therapy have facilitated the delineation of genetic characteristics that are related to either de novo/intrinsic resistance or to the development of acquired resistance in response to a variety of therapies including anti-EGFR mAbs. Use of next generation sequencing (NGS) of tumors and circulating tumor DNA (ctDNA) in plasma isolated from a peripheral blood sample has greatly enhanced understanding of tumor heterogeneity and the evaluation of multiple genomic abnormalities. Longitudinal evaluation of ctDNA during the course of treatment has also provided an opportunity to prospectively and dynamically assess the breadth and frequency of genetic abnormalities that, compared to biopsies of a single metastatic lesion, may better reflect the heterogeneity of lesions within a single patient.

Alterations in components of the RAS signaling pathway, together with mutations in the extracellular domain (ECD) of the EGFR gene, are the most common mechanisms of acquired resistance to EGFR blockade in CRC (Montagut et al., supra; Misale et al., supra; Arena et al., supra; Misale et al., Cancer Discov. 4:1269-80 (2014); Dienstmann et al., Am. Soc. Clin. Oncol. Educ. book. Am. Soc. Clin. Oncol. Meet. 35:e149-56 (2015); and Van Emburgh et al., Nat. Commun. 7:13665 (2016)). In addition, a specific mutation of the BRAF gene at amino acid position 600 (BRAF V600E) is associated with poor prognosis in CRC patients receiving cetuximab treatment (Van Cutsem et al., supra; Pietrantonio et al., Eur. J. Cancer 51:587-94 (2015)).

Some anti-EGFR-refractory cancers may be treatable by agents such as Sym004, which is a composition comprising two anti-EGFR antibodies, futuximab and modotuximab, that bind to nonoverlapping epitopes in extracellular domain III of EGFR (U.S. Pat. Nos. 7,887,805 and 8,663,640). Binding of the Sym004 mAbs to EGFR leads to highly efficient receptor internalization and degradation, which in turn leads to profound inhibition of cancer cell growth. However, there is a significant need for a means of identifying specific patient populations (e.g., anti-EGFR treatment-refractory patient populations) likely to benefit from treatment with Sym004.

### SUMMARY OF THE INVENTION

The present invention is based on the inventors' discovery of certain genetic characteristics that define a population of patients with chemorefractory mCRC and acquired resistance to approved anti-EGFR antibodies who can obtain significant clinical benefit from treatment with the anti-EGFR antibody compositions described herein. The inventors have obtained data from a randomized, sponsor-blinded, multicenter Phase 2 study of Sym004 investigating two Sym004 dose regimens vs. IC (investigator's choice; capecitabine, 5-FU, or BSC) in patients with mCRC previously treated with fluoropyrimidine-, oxaliplatin- and irinotecan-based chemotherapy and anti-Vascular Endothelial Growth Factor therapy, and with acquired resistance to anti-EGFR therapy.

The present invention provides a method for treating cancer in a patient, comprising selecting a patient with said cancer from whom a tumor DNA sample:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146);
ii) has a MAF of less than 0.1% (e.g., no detectable levels) of BRAF mutation V600E; and
iii) has a MAF of less than 0.1% (e.g., no detectable levels) of EGFR ECD mutations V441D, V441G, S464L, G465E, G465R, and S492R, and administering to the patient an anti-EGFR antibody composition comprising two anti-human EGFR antibodies that bind to distinct epitopes in the EGFR extracellular domain (ECD).

The present invention also provides a method for treating cancer in a patient, comprising selecting a patient with said cancer from whom a tumor DNA sample:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146); and
ii) has a MAF of less than 0.1% (e.g., no detectable levels) of BRAF mutation V600E,
   and administering to the patient an anti-EGFR antibody composition comprising two anti-human EGFR antibodies that bind to distinct epitopes in the EGFR extracellular domain (ECD).

In certain embodiments, the tumor DNA sample also has been determined to be negative for gene amplification of MET, ERBB2, and optionally KRAS.

In some embodiments, the cancer is selected from the group consisting of colorectal cancer, non-small cell lung cancer (NSCLC), and squamous cell carcinoma of the head and neck (SCCHN). In certain embodiments, the cancer is colorectal cancer. In particular embodiments, the cancer is metastatic colorectal cancer.

In some embodiments, the patient has received prior treatment with an anti-EGFR antibody that is not an antibody in the antibody composition (e.g., cetuximab, panitumumab, zalutumumab, nimotuzumab, ICR62, mAb806, matuzumab, or an antibody capable of binding the same epitope as any of these). In certain embodiments, the patient has been treated with cetuximab, panitumumab, or both.

In some embodiments, the cancer is resistant or partially resistant to prior treatment with an anti-EGFR antibody that is not an antibody in said antibody composition (e.g., cetuximab, panitumumab, zalutumumab, nimotuzumab, ICR62, mAb806, matuzumab, or an antibody capable of binding the same epitope as any of these). In certain embodiments, the cancer is resistant or partially resistant to prior treatment with cetuximab, panitumumab, or both. The resistance or partial resistance may be determined, e.g., by assaying a sample of cancer cells isolated from the patient.

In some embodiments, the patient has demonstrated intolerance to, or failed on prior treatment with, at least one chemotherapy agent selected from the group consisting of 5-FU, oxaliplatin, irinotecan, FOLFOX (folinic acid, fluorouracil and oxaliplatin), and FOLFIRI (folinic acid, fluorouracil and irinotecan).

In some embodiments, the tumor DNA sample may be, e.g., a circulating tumor (ct) DNA sample from the patient, or may be obtained from a tumor tissue sample or circulating tumor cells from the patient.

In some embodiments, the anti-EGFR antibody composition enhances internalization and degradation of EGFR, induces complement-dependent cytotoxicity (CDC), induces differentiation of tumor cells in vivo; and/or increases involucrin expression in vivo. In certain embodiments, the anti-EGFR antibody composition has all of these properties.

In some embodiments, the anti-EGFR antibody composition comprises a first anti-human EGFR antibody and a second anti-human EGFR antibody, wherein the first anti-human EGFR antibody comprises the heavy chain CDR1, CDR2, and CDR3 in SEQ ID NO: 1 and the light chain CDR1, CDR2, and CDR3 in SEQ ID NO: 2; and the second anti-human EGFR antibody comprises the heavy chain CDR1, CDR2, and CDR3 in SEQ ID NO: 3 and the light chain CDR1, CDR2, and CDR3 in SEQ ID NO: 4. In certain embodiments, the first anti-human EGFR antibody comprises a heavy chain comprising the amino acid sequence of SEQ ID NO: 1 and a light chain comprising the amino acid sequence of SEQ ID NO: 2; and the second anti-human EGFR antibody comprises a heavy chain comprising the amino acid sequence of SEQ ID NO: 3 and a light chain comprising the amino acid sequence of SEQ ID NO: 4. In particular embodiments, the first anti-human EGFR antibody comprises a heavy chain comprising the amino acid sequence of SEQ ID NO: 26 and a light chain comprising the amino acid sequence of SEQ ID NO: 24; and the second anti-human EGFR antibody comprises a heavy chain comprising the amino acid sequence of SEQ ID NO: 27 and a light chain comprising the amino acid sequence of SEQ ID NO: 25. The first and second anti-human EGFR antibodies of the composition may be, e.g., of isotype IgG1 or IgG2. The ratio of the first anti-human EGFR antibody relative to the second anti-human EGFR antibody may be, e.g., 1:1.

In some embodiments of the methods of the invention, the antibody composition is administered to the patient at a loading dose of 9 mg/kg, followed by a weekly dose of 6 mg/kg. In other embodiments, the antibody composition is administered to the patient at a weekly dose of 12 mg/kg. In certain embodiments, the patient is human.

In a particular embodiment, the present invention provides a method for treating cancer (e.g., metastatic colorectal cancer) in a human patient, comprising administering to the patient an anti-EGFR antibody composition comprising a first anti-human EGFR antibody that comprises a heavy chain comprising the amino acid sequence of SEQ ID NO: 26 and a light chain comprising the amino acid sequence of SEQ ID NO: 24; and a second anti-human EGFR antibody that comprises a heavy chain comprising the amino acid sequence of SEQ ID NO: 27 and a light chain comprising the amino acid sequence of SEQ ID NO: 25; wherein the antibody composition is administered intravenously to the patient at a loading dose of 9 mg/kg, followed one week later by a weekly dose of 6 mg/kg. In certain embodiments, the cancer is resistant or partially resistant to prior treatment with an anti-EGFR antibody that is not an antibody in said antibody composition (e.g., cetuximab or panitumumab).

In a particular embodiment, the present invention provides a method for treating cancer (e.g., metastatic colorectal cancer) in a human patient, comprising selecting a patient with said cancer from whom a tumor DNA sample:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146),
ii) has a MAF of less than 0.1% (e.g., no detectable levels) of BRAF mutation V600E, and
iii) has a MAF of less than 0.1% (e.g., no detectable levels) of EGFR ECD mutations V441D, V441G, S464L, G465E, G465R, and S492R;
   and administering to the patient an anti-EGFR antibody composition comprising a first anti-human EGFR antibody that comprises a heavy chain comprising the amino acid sequence of SEQ ID NO: 26 and a light chain comprising the amino acid sequence of SEQ ID NO: 24; and a second anti-human EGFR antibody that comprises a heavy chain comprising the amino acid sequence of SEQ ID NO: 27 and a light chain comprising the amino acid sequence of SEQ ID NO: 25; wherein the antibody composition is administered intravenously to the patient at a loading dose of 9 mg/kg, followed one week later by a weekly dose of 6 mg/kg. In certain embodiments, the cancer is resistant or partially resistant to prior treatment with an anti-EGFR antibody that is not an antibody in said antibody composition (e.g., cetuximab or panitumumab).

In a particular embodiment, the present invention provides a method for treating cancer (e.g., metastatic colorectal cancer) in a patient, comprising selecting a patient with said cancer from whom a tumor DNA sample:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146), and
ii) has a MAF of less than 0.1% (e.g., no detectable levels) of BRAF mutation V600E;
   and administering to the patient an anti-EGFR antibody composition comprising a first anti-human EGFR antibody that comprises a heavy chain comprising the amino acid sequence of SEQ ID NO: 26 and a light chain comprising the amino acid sequence of SEQ ID NO: 24; and a second anti-human EGFR antibody that comprises a heavy chain comprising the amino acid sequence of SEQ ID NO: 27 and a light chain comprising the amino acid sequence of SEQ ID NO: 25; wherein the antibody composition is administered intravenously to the patient at a loading dose of 9 mg/kg, followed one week later by a weekly dose of 6 mg/kg. In certain embodiments, the cancer is resistant or partially resistant to prior treatment with an anti-EGFR antibody that is not an antibody in said antibody composition (e.g., cetuximab or panitumumab).

The invention also provides the use of an antibody composition comprising two anti-human EGFR antibodies that bind to distinct epitopes in the EGFR extracellular domain (ECD) for the manufacture of a medicament for treating cancer in a patient, wherein a tumor DNA sample from the patient:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146);
ii) has a MAF of less than 0.1% (e.g., no detectable levels) of BRAF mutation V600E; and
iii) has a MAF of less than 0.1% (e.g., no detectable levels) of EGFR ECD mutations V441D, V441G, S464L, G465E, G465R, and S492R.
   The invention also provides the use of an antibody composition comprising two anti-human EGFR antibodies that bind to distinct epitopes in the EGFR extracellular domain (ECD) for the manufacture of a medicament for treating cancer in a patient, wherein a tumor DNA sample from the patient:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146); and
ii) has a MAF of less than 0.1% (e.g., no detectable levels) of BRAF mutation V600E. In some embodiments, the medicament is for treating cancer in a patient in a method of the invention. In some embodiments, the cancer is metastatic colorectal cancer. In some embodiments, the cancer is resistant or partially resistant to prior treatment with an anti-EGFR antibody that is not an antibody in said antibody composition (e.g., cetuximab or panitumumab).

The invention also provides an antibody composition comprising two anti-human EGFR antibodies that bind to distinct epitopes in the EGFR extracellular domain (ECD) for use in treating cancer in a patient in a method comprising selecting a patient with said cancer from whom a tumor DNA sample:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146);
ii) has a MAF of less than 0.1% (e.g., no detectable levels) of BRAF mutation V600E; and
iii) has a MAF of less than 0.1% (e.g., no detectable levels) of EGFR ECD mutations V441D, V441G, S464L, G465E, G465R, and S492R,
   and administering to the patient an anti-EGFR antibody composition comprising two anti-human EGFR antibodies that bind to distinct epitopes in the EGFR extracellular domain (ECD). The invention also provides an antibody composition comprising two anti-human EGFR antibodies that bind to distinct epitopes in the EGFR extracellular domain (ECD) for use in treating cancer in a patient in a method comprising selecting a patient with said cancer from whom a tumor DNA sample:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146); and
ii) has a MAF of less than 0.1% (e.g., no detectable levels) of BRAF mutation V600E,
   and administering to the patient an anti-EGFR antibody composition comprising two anti-human EGFR antibodies that bind to distinct epitopes in the EGFR extracellular domain (ECD). In some embodiments, the antibody composition is for use in treating cancer in a patient in a method of the invention. In some embodiments, the cancer is metastatic colorectal cancer. In some embodiments, the cancer is resistant or partially resistant to prior treatment with an anti-EGFR antibody that is not an antibody in said antibody composition (e.g., cetuximab or panitumumab).

The invention also provides an article of manufacture suitable for treating cancer in a patient, comprising an antibody composition comprising two anti-human EGFR antibodies that bind to distinct epitopes in the EGFR extracellular domain (ECD), wherein said treatment comprises selecting a patient with said cancer from whom a tumor DNA sample:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146);
ii) has a MAF of less than 0.1% (e.g., no detectable levels) of BRAF mutation V600E; and
iii) has a MAF of less than 0.1% (e.g., no detectable levels) of EGFR ECD mutations V441D, V441G, S464L, G465E, G465R, and S492R,
   and administering to the patient the antibody composition. The invention also provides an article of manufacture suitable for treating cancer in a patient, comprising an antibody composition comprising two anti-human EGFR antibodies that bind to distinct epitopes in the EGFR extracellular domain (ECD), wherein said treatment comprises selecting a patient with said cancer from whom a tumor DNA sample:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146); and
ii) has a MAF of less than 0.1% (e.g., no detectable levels) of BRAF mutation V600E,
   and administering to the patient the antibody composition. In some embodiments, the article is suitable for treating cancer in a patient in a method of the invention. In some embodiments, the cancer is metastatic colorectal cancer. In some embodiments, the cancer is resistant or partially resistant to prior treatment with an anti-EGFR antibody that is not an antibody in said antibody composition (e.g., cetuximab or panitumumab).

The invention also provides a kit suitable for treating cancer in a patient from whom a tumor DNA sample:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146);
ii) has a MAF of less than 0.1% (e.g., no detectable levels) of BRAF mutation V600E; and
iii) has a MAF of less than 0.1% (e.g., no detectable levels) of EGFR ECD mutations V441D, V441G, S464L, G465E, G465R, and S492R,
   wherein the kit comprises an antibody composition comprising two anti-human EGFR antibodies that bind to distinct epitopes in the EGFR extracellular domain (ECD). The invention also provides a kit suitable for treating cancer in a patient from whom a tumor DNA sample:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146); and
ii) has a MAF of less than 0.1% (e.g., no detectable levels) of BRAF mutation V600E,
   wherein the kit comprises an antibody composition comprising two anti-human EGFR antibodies that bind to distinct epitopes in the EGFR extracellular domain (ECD). In some embodiments, the kit is suitable for treating cancer in a patient in a method of the invention. In some embodiments, the cancer is metastatic colorectal cancer. In some embodiments, the cancer is resistant or partially resistant to prior treatment with an anti-EGFR antibody that is not an antibody in said antibody composition (e.g., cetuximab or panitumumab).

In some embodiments of the uses, antibody compositions, articles of manufacture, and kits described above, the antibody composition comprises a first anti-human EGFR antibody and a second anti-human EGFR antibody, wherein:

the first anti-human EGFR antibody comprises the heavy chain CDR1, CDR2, and CDR3 in SEQ ID NO: 1 and the light chain CDR1, CDR2, and CDR3 in SEQ ID NO: 2; and

the second anti-human EGFR antibody comprises the heavy chain CDR1, CDR2, and CDR3 in SEQ ID NO: 3 and the light chain CDR1, CDR2, and CDR3 in SEQ ID NO: 4.

In some embodiments of the uses, antibody compositions, articles of manufacture, and kits described above, the antibody composition comprises a first anti-human EGFR antibody and a second anti-human EGFR antibody, wherein:

the first anti-human EGFR antibody comprises a heavy chain comprising the amino acid sequence of SEQ ID NO: 1 and a light chain comprising the amino acid sequence of SEQ ID NO: 2; and

the second anti-human EGFR antibody comprises a heavy chain comprising the amino acid sequence of SEQ ID NO: 3 and a light chain comprising the amino acid sequence of SEQ ID NO: 4.

In some embodiments of the uses, antibody compositions, articles of manufacture, and kits described above, the antibody composition comprises a first anti-human EGFR antibody and a second anti-human EGFR antibody, wherein:

the first anti-human EGFR antibody comprises a heavy chain comprising the amino acid sequence of SEQ ID NO: 26 and a light chain comprising the amino acid sequence of SEQ ID NO: 24; and

the second anti-human EGFR antibody comprises a heavy chain comprising the amino acid sequence of SEQ ID NO: 27 and a light chain comprising the amino acid sequence of SEQ ID NO: 25.

It is understood that any of the antibody compositions and antibodies and antigen-binding portions thereof described herein may be used in any method of treatment as described herein, may be for use in any treatment as described herein, and/or may be for use in the manufacture of a medicament for any treatment as described herein.

Other features and advantages of the invention will be apparent from the following description and embodiments.

### BRIEF DESCRIPTION OF THE DRAWINGS

FIGS. 1-5 depict analysis of genetic alterations detected in ctDNA from anti-EGFR refractory metastatic colorectal cancer patients.

FIG. 1 is a graph depicting the number of genetic alterations (single nucleotide variants, copy number variants, indels, and fusions) identified in ctDNA from patients (N=193), listed by gene.

FIG. 2 is a graph depicting mutant allele frequency (MAF) of APC+TP53; KRAS exon 2 (codons 12 and 13), exon 3 (codons 59 and 61), and exon 4 (codons 117 and 146); NRAS exon 2 (codons 12 and 13), exon 3 (codons 59 and 61), and exon 4 (codons 117 and 146); BRAF V600E; and the EGFR extracellular domain (ECD) mutations V441D, V441G, S464L, G465E, G465R, and S492R, in the 193 patients. For patients with more than one alteration in the same gene, only the highest MAF alteration for each gene is shown. Lines denote the median, black bars denote the interquartile range, and the dotted line depicts MAF=20%.

FIGS. 3A-3D depict lollipop plots of missense mutations identified in the EGFR (FIG. 3A), BRAF (FIG. 3B), KRAS (FIG. 3C), and NRAS (FIG. 3D) genes in the current study (top half of each plot) compared to data obtained from The Cancer Genome Atlas (TCGA) (bottom half of each plot). Amino acid alterations detected at mutational hotspots are depicted for each gene.

FIG. 4 is a pie chart showing patient distribution and frequency of EGFR single nucleotide variant missense mutations. N: Number of patients with each mutation; %: Percentage of the total number of non-silent single nucleotide variant mutations in EGFR detected in the patients.

FIG. 5 is a bar graph depicting number of genetic alterations for each genetically profiled patient, grouped by patients with EGFR ECD mutations to the left (box). The oncoprint denotes the six individual EGFR ECD mutations (V441D, V441G, S464L, G465E, G465R, and S492R), KRAS mutations in exon 2, 3, or 4 at all MAFs (KRAS) and at MAF>20% (KRAS>20), NRAS mutations in exon 2, 3, or 4 at all MAFs (NRAS) and at MAF>20% (NRAS>20), MET and ERBB2 gene amplifications (defined as copy number >5), and BRAF V600E. % denotes the fraction of patients harboring the defined alteration.

FIGS. 6-11 depict the binding and functional activity of Sym004 in cell lines harboring EGFR ECD mutations.

FIG. 6 is a heat map showing how the binding of different antibodies is affected by the different EGFR ECD mutations. The effect on binding was determined as the fold-reduction in EC₅₀ relative to WT EGFR.

FIG. 7 is a graph showing quantification of basal and EGF induced phosphorylated EGFR (pEGFR) levels in NIH-3T3 cells stably overexpressing WT or mutant EGFR by Simple Western analysis. pEGFR and total EGFR signal intensities were normalized to pan-actin (loading control). Data are presented as ratios.

FIG. 8 is a series of graphs showing dose-response curves showing the effect of the indicated antibodies on cell viability in NIH-3T3 cells stably overexpressing WT (Panel A) or mutant (Panels B-E) EGFR. Each data point represents the mean of three replicates±Standard Deviation (SD).

FIG. 9 is a series of graphs showing the ability of Sym004 to block ligand induced phosphorylation of EGFR in NIH-3T3 cells transiently transfected with either WT or mutant EGFR. Cells were cultured in the presence of the indicated antibodies for 4 hours and stimulated with 1 nM EGF for 10 minutes. pEGFR (Tyr1068) levels were determined by Simple Western analysis. The pEGFR signal intensity was normalized to pan-actin (loading control) and is presented as a percentage of the signal in unstimulated control cells. Each bar represents the mean of three replicates. Error bars represent SD.

FIG. 10 is a pair of graphs showing dose-response curves depicting the effect of the indicated antibodies on cell viability in DiFi (left) and DCR7 (right) cells stably overexpressing WT or S492R mutant EGFR, respectively. Each data point represents the mean of three replicates±SD.

FIG. 11 is a pair of graphs showing tumor growth curves upon treatment with Sym004 in BALB/c nude mice xenotransplanted with EGFR WT DiFi (left) or S492R EGFR-mutant DCR7 (right) cells. Tumor volumes were normalized individually to their volumes on the first day of treatment. Cetuximab and Sym004 significantly reduced tumor growth in DiFi injected mice (P<0.0001). In S492R EGFR-mutant DiFi injected mice Sym004 significantly suppressed tumor growth (P<0.0001).

FIG. 12 is a bar graph depicting the number of patients with each of the six EGFR ECD mutations who were treated with either cetuximab or panitumumab as the last anti-EGFR treatment prior to study enrollment.

FIG. 13 is a graph showing the dynamics of mutant allele frequencies (MAFs) of EGFR ECD mutations detected in serum samples obtained prior to treatment (week 1) and at week 3 of treatment with Sym004. For each EGFR ECD mutation, the EGFR ECD MAF was normalized to the TP53 MAF.

FIG. 14 is a graph showing EGFR ECD mutation dynamics in two patients (black and gray), demonstrating an increase in MAF for one EGFR ECD mutation and a decreased MAF for one or more other EGFR ECD mutations.

FIG. 15 shows Venn diagrams depicting the number (fraction of all profiled patients in parentheses) of patients harboring concurrent mutations in the EGFR ECD (V441D, V441G, S464L, G465E, G465R, and S492R) and KRAS/NRAS exons 2, 3, and 4 (RAS), as well as BRAF V600E, at various mutant allele frequencies (MAFs): RAS ALL MAF, RAS>1% MAF, RAS>5% MAF, and RAS>20% MAF.

FIGS. 16A-16C show bar graphs depicting overall survival (OS) for all genetically profiled patients. Patients are grouped by treatment and sorted by increasing OS from left to right. The oncoprints denote patients with EGFR ECD mutations (G465R, G465E, S464L, S492R, V441D, and V441G), KRAS mutations in exon 2, 3, or 4 at all MAFs (KRAS) and at MAF>20% (KRAS MAF>20%), NRAS mutations in exon 2, 3, or 4 at all MAFs (NRAS) and at MAF>20% (NRAS MAF>20%), MET and ERBB2 gene amplifications (copy number >5), and BRAF V600E.

FIG. 17 is a graph depicting Kaplan-Meier curves of overall survival in patients with DN (double negative) mCRC.

FIG. 18 is a graph depicting Kaplan-Meier curves of overall survival in patients with TN (triple negative) mCRC.

FIG. 19 depicts an overview of the 70 genes included in the Guardant360 version 2.9 panel. Genes were sequenced in critical exon regions except for those highlighted in bold, where the full exon was sequenced.

FIG. 20 is a series of bar graphs showing levels of specific lysis of NIH3T3 cells stably expressing EGFR WT or the indicated EGFR ECD mutants induced by futuximab, modotuximab, Sym004, cetuximab, or panitumumab (5 μg/mL) in the presence of human primary NK cells (ADCC assay). Each bar represents the mean of four replicates. Error bars represent SEM.

FIG. 21 is a series of bar graphs showing total EGFR levels for WT EGFR or the indicated EGFR ECD mutants after 48 hours of treatment with the indicated antibodies, as determined by Simple Western analysis. EGFR signal intensity was normalized to pan-actin (loading control) and is presented as a percentage of the signal in untreated control cells. Each bar represents the mean of three replicates. Error bars represent SD.

FIG. 22 is a bar graph showing the fraction of patients with each EGFR ECD mutation who had previously been treated with cetuximab, panitumumab, or both prior to enrollment.

FIG. 23 is a graph showing the number of genetic alterations in all ctDNA profiled patients compared to patients harboring EGFR ECD mutations.

FIG. 24 shows Venn diagrams depicting the number (fraction of all profiled patients in parentheses) of patients harboring concurrent mutations in the EGFR ECD (G465E, G465R, S464L, S492R, V441D, and V441G) and KRAS/NRAS exons 2, 3, and 4 (RAS), as well as BRAF V600E, at various mutant allele frequencies (MAFs): RAS ALL MAF, RAS>1% MAF, RAS>2% MAF, RAS>3% MAF, RAS>4% MAF, RAS>5% MAF, RAS>10% MAF, RAS>20% MAF, RAS>25% MAF, and RAS>50% MAF.

FIGS. 25-27 show graphs depicting examples of tumor growth curves in animals treated with vehicle (black circle), cetuximab (black square), or Sym004 (white circle) (30 mg/kg i.p. twice weekly). The gray area marks the treatment period.

FIG. 28 depicts in the top panel a waterfall plot showing tumor growth response at day 28, or the closest day to day 28, in 36 CRC PDX models treated with cetuximab (white) or Sym004 (black). PD: Progressive disease; SD: Stable disease; PR/CR: Partial response/complete response. The bottom panel shows mutations found in the PDX models: KRAS (top), NRAS (middle), and BRAF (bottom).

FIGS. 29-31 show oncoprints depicting the full ctDNA profiles of patients treated with Sym004 12 mg/kg (FIG. 29), Sym004 9/6 mg/kg (FIG. 30), or investigator's choice (FIG. 31). For all figures, the patients are sorted by overall survival, with poorest performing patients to the left. % denotes the fraction of patients in the treatment group with alterations in the specific gene. Amplifications are defined as more than five copies; gain is defined as copy number of more than 2.2 and less than five copies.

FIG. 32 is a box-and-whisker (5-95% percentile) plot of EGFR ECD MAF detected in ctDNA from anti-EGFR refractory mCRC patients.

FIG. 33 is a box-and-whisker (5-95% percentile) plot of EGFR ECD MAF detected in reference tumors from 8 anti-EGFR refractory mCRC patients.

### DETAILED DESCRIPTION OF THE INVENTION

The present invention is based on the inventors' discovery that the anti-human EGFR antibody compositions described herein, such as Sym004, are effective in treating cancer patients negative for certain RAS and BRAF mutations, or negative for certain RAS, BRAF, and EGFR ECD mutations. Some of these patients may have developed resistance to treatment with anti-EGFR antibodies cetuximab and panitumumab, but will respond to the present therapeutic composition. As described in the Examples, the inventors evaluated the efficacy of Sym004 in chemorefractory metastatic colorectal cancer (mCRC) patients with acquired resistance to EGFR blockade. Treatment with Sym004 (12 mg/kg weekly (arm A) or a 9 mg/kg loading dose followed by 6 mg/kg weekly (arm B)) did not demonstrate a clinical benefit over standard treatment with capecitabine, 5-FU, or best supportive care (BSC) (arm C) in that general patient population. Median overall survival (OS) in the population was 7.9 months (95% confidence interval [CI] 6.5-9.9), 10.3 months (95% CI 9-12.9), and 9.6 months (95% CI 8.3-12.2) for arms A, B, and C, respectively. The one-year survival rate was 37% (95% CI 26-47), 44% (95% CI 33-54%), and 40% (95% CI 29-51%), for arms A, B, and C, respectively. However, the inventors have found that Sym004 was surprisingly effective in treating mCRC tumors resistant to treatment with other anti-EGFR antibodies in patients who are negative for the above resistance-causing mutations (i.e., patients in whom the mechanism of resistance is unknown). Unpredictably, Sym004 succeeded in this population where cetuximab and panitumumab have failed. This discovery allows for accurate selection of mCRC patients who have developed resistance to known EGFR inhibitors such as cetuximab and panitumumab for a further line treatment with Sym004. Accordingly, detection of these RAS, BRAF, and EGFR ECD mutations and/or their frequency in treatment-refractory mCRC patients will be paramount in designing additional lines of therapy that include Sym004.

Sym004 is a 1:1 mixture of two recombinant, human-mouse chimeric mAbs directed against nonoverlapping EGFR epitopes. A unique feature of Sym004 is its ability to mediate rapid EGFR internalization and subsequent degradation of the receptor (Pedersen et al., Cancer Res. 70:588-597 (2010) and Koefoed et al., MAbs 3:584-95 (2011)). Preclinical studies with Sym004 showed superior antitumor activity as compared with other anti-EGFR antibodies as well as activity in models of acquired cetuximab resistance (Pedersen et al., supra, and lida et al., Neoplasia 15:1196-206 (2013)). Sym004 has shown promising responses in a phase I clinical trial involving mCRC patients with disease resistant or refractory to cetuximab and/or panitumumab. Sym004 is further described in PCT Patent Publications WO 2008/104183 and WO 2010/022736 (hereby incorporated by reference in their entirety) as the combination of antibodies 992 (futuximab) and 1024 (modotuximab). The invention also contemplates the use of other antibody compositions (e.g., as described in U.S. Patent Publication 2012/0308576; Kearns et al., Mol Cancer Ther 14(7):1625-1636 (2015); and Arena et al., Science Translational Medicine 8(324):324ra14 (2016)); hereby incorporated by reference).

### Antibody-Related Definitions

The term “antibody” (Ab) or “immunoglobulin” (Ig), as used herein, refers to a tetramer comprising two heavy (H) chains (about 50-70 kDa) and two light (L) chains (about 25 kDa) interconnected by disulfide bonds. Each heavy chain is comprised of a heavy chain variable domain (VH) and a heavy chain constant region (CH). Each light chain is composed of a light chain variable domain (VL) and a light chain constant region (CL). The VH and VL domains can be subdivided further into regions of hypervariability, termed “complementarity determining regions” (CDRs), interspersed with regions that are more conserved, termed “framework regions” (FRs). Each VH and VL is composed of three CDRs (H-CDR herein designates a CDR from the heavy chain; and L-CDR herein designates a CDR from the light chain) and four FRs, arranged from amino-terminus to carboxyl-terminus in the following order: FR1, CDR1, FR2, CDR2, FR3, CDR3, FR4. The assignment of amino acid numbers in the heavy or light chain may be in accordance with IMGT® definitions (Lefranc et al., Dev Comp Immunol 27(1):55-77 (2003)); or the definitions of Kabat, Sequences of Proteins of Immunological Interest (National Institutes of Health, Bethesda, Md. (1987 and 1991)); Chothia & Lesk, J. Mol. Biol. 196:901-917 (1987); or Chothia et al., Nature 342:878-883 (1989). Unless otherwise indicated, all antibody amino acid residue numbers referred to in this disclosure are those under the IMGT® numbering scheme.

The term “recombinant antibody” refers to an antibody that is expressed from a cell or cell line comprising the nucleotide sequence(s) that encode the antibody, wherein said nucleotide sequence(s) are not naturally associated with the cell.

The term “anti-EGFR antibody composition” refers to a composition comprising at least one anti-EGFR antibody as described herein or an antigen-binding portion thereof.

The term “isolated protein,” “isolated polypeptide,” or “isolated antibody” refers to a protein, polypeptide or antibody, respectively, that by virtue of its origin or source of derivation (1) is not associated with naturally associated components that accompany it in its native state, (2) is free of other proteins from the same species, (3) is expressed by a cell from a different species, and/or (4) does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The term “epitope” as used herein refers to a portion (determinant) of an antigen that specifically binds to an antibody or a related molecule such as a bispecific binding molecule. Epitopic determinants generally consist of chemically active surface groupings of molecules such as amino acids or carbohydrate or sugar side chains and generally have specific three-dimensional structural characteristics, as well as specific charge characteristics. An epitope may be “linear” or “conformational.” In a linear epitope, all of the points of interaction between a protein (e.g., an antigen) and an interacting molecule (such as an antibody) occur linearly along the primary amino acid sequence of the protein. In a conformational epitope, the points of interaction occur across amino acid residues on the protein that are separated from one another in the primary amino acid sequence. Once a desired epitope on an antigen is determined, it is possible to generate antibodies to that epitope using techniques well known in the art. For example, an antibody to a linear epitope may be generated, e.g., by immunizing an animal with a peptide having the amino acid residues of the linear epitope. An antibody to a conformational epitope may be generated, e.g., by immunizing an animal with a mini-domain containing the relevant amino acid residues of the conformational epitope. An antibody to a particular epitope can also be generated, e.g., by immunizing an animal with the target molecule of interest or a relevant portion thereof, then screening for binding to the epitope.

One can determine whether an antibody binds to the same epitope as, or competes for binding with, an anti-EGFR antibody as described herein by using methods known in the art, including, without limitation, competition assays, epitope binning, and alanine scanning. In one embodiment, one allows the anti-EGFR antibody as described herein to bind to EGFR under saturating conditions and then measures the ability of the test antibody to bind to EGFR. If the test antibody is able to bind to EGFR at the same time as the reference anti-EGFR antibody, then the test antibody binds to a different epitope than the reference anti-EGFR antibody. However, if the test antibody is not able to bind to EGFR at the same time, then the test antibody binds to the same epitope, an overlapping epitope, or an epitope that is in close proximity to the epitope bound by the anti-EGFR antibody as described herein. This experiment can be performed using, e.g., ELISA, RIA, BIACORE™ SPR, Bio-Layer Interferometry or flow cytometry. To test whether an anti-EGFR antibody cross-competes with another anti-EGFR antibody, one may use the competition method described above in two directions, i.e., determining if the known antibody blocks the test antibody and vice versa. Such cross-competition experiments may be performed, e.g., using an IBIS MX96 SPR instrument or the Octet™ system.

Examples of antibodies binding to the same epitope as antibody 992 are antibodies 1209, 1204, 996, 1033, and 1220 as defined in PCT Patent Publication WO 2010/022736 (incorporated herein by reference). Examples of antibodies binding to the same epitope as antibody 1024 are antibodies 1031, 1036, 1042, 984, 1210, 1217, 1221, and 1218 as defined in PCT Patent Publication WO 2010/022736.

The term “antigen-binding portion” of an antibody (or simply “antibody portion”), as used herein, refers to one or more portions or fragments of an antibody that retain the ability to specifically bind to an antigen (e.g., human EGFR, or a portion thereof). It has been shown that certain fragments of a full-length antibody can perform the antigen-binding function of the antibody. Examples of binding fragments encompassed within the term “antigen-binding portion” include (i) a Fab fragment: a monovalent fragment consisting of the V_{L}, V_{H}, C_{L} and C_{H}1 domains; (ii) a F(ab′)₂ fragment: a bivalent fragment comprising two Fab fragments linked by a disulfide bridge at the hinge region; (iii) an Fd fragment consisting of the V_{H} and C_{H}1 domains; (iv) a Fv fragment consisting of the V_{L} and V_{H} domains of a single arm of an antibody, (v) a dAb fragment, which consists of a V_{H} domain; and (vi) an isolated complementarity determining region (CDR) capable of specifically binding to an antigen. Furthermore, although the two domains of the Fv fragment, V_{L} and V_{H}, are encoded by separate genes, they can be joined, using recombinant methods, by a synthetic linker that enables them to be made as a single protein chain in which the V_{L} and V_{H} domains pair to form monovalent molecules (known as single chain Fv (scFv)). Also within the invention are antigen-binding molecules comprising a V_{H} and/or a V_{L}. In the case of a V_{H}, the molecule may also comprise one or more of a CH1, hinge, CH2, or CH3 region. Such single chain antibodies are also intended to be encompassed within the term “antigen-binding portion” of an antibody. Other forms of single chain antibodies, such as diabodies, are also encompassed. Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen-binding sites.

Antibody portions, such as Fab and F(ab′)₂ fragments, can be prepared from whole antibodies using conventional techniques, such as papain or pepsin digestion of whole antibodies. Moreover, antibodies, antibody portions and immunoadhesion molecules can be obtained using standard recombinant DNA techniques, e.g., as described herein.

The class (isotype) and subclass of anti-EGFR antibodies described herein may be determined by any method known in the art. In general, the class and subclass of an antibody may be determined using antibodies that are specific for a particular class and subclass of antibody. Such antibodies are available commercially. The class and subclass can be determined by ELISA and Western Blot as well as other techniques. Alternatively, the class and subclass may be determined by sequencing all or a portion of the constant regions of the heavy and/or light chains of the antibodies, comparing their amino acid sequences to the known amino acid sequences of various classes and subclasses of immunoglobulins, and determining the class and subclass of the antibodies. A preferred isotype of the present invention is an IgG isotype.

### Patient Populations

The invention provides means and materials for treating specific patient populations with a disorder such as cancer using an anti-EGFR antibody composition. The invention also provides means of selecting patients for such treatment.

In some embodiments, the cancer is selected from the group consisting of cancer of the bladder, breast, uterus/cervix, colon, kidney, ovary, prostate, renal cell, pancreas, colon, rectum, stomach, squamous cell, lung (non-small cell), esophagus, head and neck, and skin. In particular embodiments, the cancer is metastatic colorectal cancer (mCRC).

In some embodiments, the patient is resistant or partially resistant to therapy with an anti-EGFR antibody, e.g., an antibody to the extracellular domain of EGFR. In certain embodiments, the anti-EGFR antibody is selected from the group consisting of cetuximab, panitumumab, zalutumumab, nimotuzumab, ICR62, mAb806, matuzumab, and anti-EGFR antibodies capable of binding the same epitope as any of these. In certain embodiments, the antibody is selected from the group consisting of cetuximab, panitumumab, and zalutumumab (which bind to the same epitope of EGFR). In particular embodiments, the antibody is cetuximab, panitumumab, or both. In some embodiments, the patient has acquired resistance to the anti-EGFR antibody therapy.

Symptoms that may be associated with resistance include, for example, a decline in the well-being of the patient, an increase in the size of a tumor or in the rate of tumor growth, and/or spread of cancer cells from one location in the body to other organs or tissues. Symptoms may also include an increase in EGFR activity (e.g., EGFR overexpression or hyperactivity).

The patient optionally also has demonstrated prior intolerance to or failure of a chemotherapeutic regimen. In some embodiments, the chemotherapeutic regimen comprises or consists of 5-fluorouracil (5-FU), oxaliplatin, or irinotecan, or any combination of said agents. In some embodiments, the patient has not received prior therapy with regorafenib and/or trifluridine/tipiracil (TAS-102).

In some embodiments, a tumor DNA sample from the patient is negative for certain mutations in the RAS and BRAF genes, or the RAS, BRAF, and EGFR ECD genes.

The term “RAS” includes KRAS and NRAS. The amino acid sequence of human KRAS may be found at SwissProt Accession No. P01116 (SEQ ID NO: 28). The amino acid sequence of human NRAS may be found at SwissProt Accession No. P01111 (SEQ ID NO: 29).

The term “BRAF” refers to serine/threonine-protein kinase B-raf. The amino acid sequence of human BRAF may be found at SwissProt Accession No. P15056 (SEQ ID NO: 30).

The terms “epidermal growth factor receptor” and “EGFR” are used interchangeably herein. The amino acid sequence of mature human EGFR may be found at SwissProt Accession Number P00533 (SEQ ID NO: 31). The extracellular domain (ECD) of EGFR is generally considered to consist of four sub-domains, and is found at residues 25-645 of SEQ ID NO: 31.

As used herein, the tumor DNA sample may be considered “negative for” RAS mutations if the following mutations are detected in the sample at a mutant allele frequency (MAF) of less than 20% (i.e., if in a patient sample, less than 20% of the tumor DNA analyzed for a specific gene contains the mutation): mutations in KRAS exon 2 (codons 12 and 13), exon 3 (codons 59 and 61), and exon 4 (codons 117 and 146); and mutations in NRAS exon 2 (codons 12 and 13), exon 3 (codons 59 and 61), and exon 4 (codons 117 and 146). The codons correspond to the amino acid residues at the recited positions of the protein sequence (e.g., “codon 12” of KRAS corresponds to residue 12 of SEQ ID NO: 28).

In certain embodiments, a tumor DNA sample from a patient selected for treatment with the methods of the invention has a MAF<20% for a KRAS mutation at residue 12 (e.g., G12A/C/D/F/R/V), residue 59 (e.g., A59E/G/T), residue 61 (e.g., Q61H/K/L), residue 117 (e.g., K117N), residue 146 (e.g., A146T/P/V), or any combination thereof. In certain embodiments, a tumor DNA sample from a patient selected for treatment with the methods of the invention has a MAF<20% for a NRAS mutation at residue 12 (e.g., G12A/C/D/R/S/V), residue 61 (e.g., Q61H/K/L/R), residue 117 (e.g., K117N), residue 146 (e.g., A146T), or any combination thereof.

In certain embodiments, a tumor DNA sample from a patient selected for treatment with the methods of the invention has a MAF of less than 50%, 40%, 30%, 25%, 20%, 15%, 10%, 5%, 4%, 3%, 2%, 1%, 0.5%, 0.1%, 0.01%, 0.005%, 0.001%, or 0.0001% (e.g., a MAF of less than 20%), or undetectable levels, for mutations in KRAS exon 2 (e.g., codons 12 and 13), exon 3 (e.g., codons 59 and 61), and/or exon 4 (e.g., codons 117 and 146); mutations in NRAS exon 2 (e.g., codons 12 and 13), exon 3 (e.g., codons 59 and 61), and/or exon 4 (e.g., codons 117 and 146); or any combination thereof. Any combination of MAFs and RAS mutations is also contemplated.

In some embodiments, the tumor DNA sample may be considered “negative for” a BRAF mutation if the V600E mutation is detected in the sample at a MAF of less than 0.1%, less than 0.01%, less than 0.005%, less than 0.001%, or less than 0.0001%. In some embodiments, the tumor DNA sample is considered “negative for” a BRAF mutation if the V600E mutation cannot be detected in the sample.

In some embodiments, a tumor DNA sample from a patient selected for treatment with the methods of the invention has a MAF of less than 50%, 40%, 30%, 25%, 20%, 15%, 10%, 5%, 4%, 3%, 2%, 1%, 0.5%, 0.1%, 0.01%, 0.005%, 0.001%, or 0.0001%, or undetectable levels, for the BRAF V600E mutation.

In some embodiments, the tumor DNA sample may be considered “negative for” EGFR ECD mutations if the V441D, V441G, S464L, G465E, G465R, and S492R mutations are detected in the sample at a MAF of less than 0.1%, less than 0.01%, less than 0.005%, less than 0.001%, or less than 0.0001%. In some embodiments, the tumor DNA sample is considered “negative for” EGFR ECD mutations if the V441D, V441G, S464L, G465E, G465R, and S492R mutations cannot be detected in the sample.

In some embodiments, a tumor DNA sample from a patient selected for treatment with the methods of the invention has a MAF of less than 50%, 40%, 30%, 25%, 20%, 15%, 10%, 5%, 4%, 3%, 2%, 1%, 0.5%, 0.1%, 0.01%, 0.005%, 0.001%, or 0.0001%, or undetectable levels, for one, two, three, four, five, or all six of said EGFR ECD mutations; any combination thereof is also contemplated. In certain embodiments, the tumor DNA sample has undetectable levels of one, two, three, four, five, or all six of said EGFR ECD mutations. In some embodiments, the tumor DNA sample may also have a MAF of less than 50%, 40%, 30%, 25%, 20%, 15%, 10%, 5%, 4%, 3%, 2%, 1%, 0.5%, 0.1%, 0.01%, 0.005%, 0.001%, or 0.0001%, or undetectable levels, for EGFR ECD mutations F404V, S442R, G465V, or I491R.

In some embodiments, a tumor DNA sample from a patient selected for treatment with the methods of the invention has a MAF of less than 50%, 40%, 30%, 25%, 20%, 15%, 10%, 5%, 4%, 3%, 2%, 1%, 0.5%, 0.1%, 0.01%, 0.005%, 0.001%, or 0.0001%, or undetectable levels, for mutations at positions 441, 464, 465, and 492 of EGFR; any combination thereof is also contemplated. In certain embodiments, the tumor DNA sample has undetectable levels of mutations at one, two, three, or all four of said positions.

In certain embodiments, a tumor DNA sample from a patient selected for treatment with the methods of the invention will have any MAF(s) for RAS mutations as described herein and any MAF for BRAF mutation V600E as described herein. In certain embodiments, a tumor DNA sample from a patient selected for treatment with the methods of the invention will have any MAF(s) for RAS mutations as described herein, any MAF for BRAF mutation V600E as described herein, and any MAF(s) for EGFR ECD mutations as described herein.

In some embodiments, the tumor DNA sample from the patient is negative for RAS mutations (i.e., has a MAF of less than 20% for mutations in KRAS exon 2 (codons 12 and 13), exon 3 (codons 59 and 61), and/or exon 4 (codons 117 and 146) and mutations in NRAS exon 2 (codons 12 and 13), exon 3 (codons 59 and 61), and/or exon 4 (codons 117 and 146)), and is negative for (i.e., has a MAF of less than 0.1% for, e.g., does not show detectable levels of) BRAF mutation V600E. In certain embodiments, the tumor DNA sample is also negative for (i.e., has a MAF of less than 0.1% for, e.g., does not show detectable levels of) EGFR ECD mutations V441D, V441G, S464L, G465E, G465R, and S492R.

In some embodiments, the tumor DNA sample from the patient is negative for (e.g., shows no detectable levels of, or has a copy number of <2, 3, 4, or 5 for) gene amplification of MET, ERBB2, KRAS, or any combination thereof (e.g., MET and ERBB2).

A tumor DNA sample refers to circulating tumor DNA (ctDNA) or DNA obtained from a tumor sample from the patient. A tumor sample can be, for example, a tumor biopsy (i.e., a biopsy of the tumor tissue) or a liquid biopsy (i.e., circulating tumor cells). Tumor DNA from a patient may be genotyped for RAS, BRAF, and/or EGFR ECD mutations, as well as any other genetic alterations discussed herein, by any method known in the art. Any suitable biological sample with tumor DNA from the patient may be used for genotype analysis, including, for example, a body fluid sample (e.g., plasma with ctDNA), cell sample, tissue sample, or circulating tumor cells (CTCs). Suitable body fluids include, but are not limited to, pleural fluid samples, pulmonary or bronchial lavage fluid samples, synovial fluid samples, peritoneal fluid samples, bone marrow aspirate samples, lymph, cerebrospinal fluid, ascites fluid samples, amniotic fluid samples, sputum samples, bladder washes, semen, urine, saliva, tears, blood and its components (serum and plasma), and the like. Cell samples obtained from a tumor biopsy or resection may also be used. In certain embodiments, a plasma sample may be obtained from the patient and ctDNA in the plasma may be tested for RAS, BRAF, and/or EGFR ECD mutations, as well as any other genetic alterations discussed herein. In certain embodiments, a sample of tumor tissue may be obtained from the patient and tested for RAS, BRAF, and/or EGFR ECD mutations, as well as any other genetic alterations discussed herein. In certain embodiments, a blood sample may be obtained from the patient and circulating tumor cells isolated from the blood sample may be tested for RAS, BRAF, and/or EGFR ECD mutations or other genetic alterations discussed herein.

Any method known in the art may be used to detect RAS, BRAF, and EGFR ECD mutations, or other genetic alterations discussed herein, in a tumor DNA sample (e.g., a ctDNA sample), including methods involving analysis of a nucleic acid (either DNA or RNA) or analysis of a protein product. In some embodiments, the mutations or genetic alterations are detected using next generation sequencing, a high-throughput technology where large numbers of DNA fragments are sequenced in parallel (e.g., Guardant360™, Illumina (Solexa) sequencing, Roche 454 sequencing, ion torrent: proton/PGM sequencing, or SOLiD sequencing). Mutant allele frequency then can be calculated by methods known in the art. For example, next generation sequencing may provide hundreds or thousands of reads at a given genomic position; the frequency of mutant alleles then can be calculated by dividing the number of times a mutant allele is detected by the total number of reads at that position (see, e.g., Sallman et al., Hematol Oncol Stem Cell Ther 9:89-95 (2016)).

In some embodiments, a mutation may be detected by contacting a nucleic acid sample with a probe that is capable of specifically hybridizing to the mutant sequence and then detecting hybridization of the probe. The probe generally is detectably labeled, such as with a radioisotope, a fluorescent agent, or a chromogenic agent to facilitate detection of hybridization. The skilled worker would readily be able to design a suitable probe for detecting a mutation of interest, e.g., a RAS, BRAF, or EGFR ECD mutation described herein.

Detection of point mutations may also be accomplished by molecule cloning and sequencing of polynucleotides using techniques well known in the art. Polymerase chain reaction (PCR) can also be used to amplify gene sequences directly from a genomic DNA preparation from a biological sample, e.g., ctDNA-containing plasma, a tumor tissue sample or circulating tumor cell sample. In some embodiments, the PCR may be digital droplet PCR. The DNA sequence of the amplified sequences can then be determined and mutations identified.

Mismatch detection (e.g., detection of duplexes that are not 100% complementary) can also be used to detect point mutations in a DNA or RNA sequence. RNase protection, which involves mismatch cleavage, is another means of detection mutations, including point mutations. This method involves use of a labeled riboprobe which is complementary to a wild-type sequence. The riboprobe and either mRNA or DNA isolated from a sample are annealed (hybridized) together and subsequently digested with enzyme RNase A, which is able to detect some mismatches in duplex RNA structure and cleave at the mismatch site. The cleaved fragments can be detected using, e.g., gel electrophoresis.

### Anti-EGFR Antibody Compositions

The antibody compositions used in the present invention may comprise mAbs targeting non-overlapping epitopes of EGFR. An exemplary composition is Sym004, a mixture of two mAbs, 992 (futuximab) and 1024 (modotuximab), that bind to non-overlapping epitopes in EGFR extracellular domain (ECD) III. Sym004 represents a differentiated antibody product targeting the EGFR pathway with documented superiority over the clinically approved antibodies cetuximab and panitumumab in preclinical studies (Sanchez-Martin et al., supra; Pedersen et al., supra; and lida et al., supra). As discussed in PCT Patent Publication WO 2008/104183, Sym004 treatment provides a novel mechanism of action involving terminal differentiation accompanied by increased involucrin expression and the appearance of keratin pearls in an animal model. Further, in vivo studies have shown that tumors continue to diminish after termination of Sym004 treatment; by contrast, in a control group receiving Erbitux, tumors start growing soon after termination of treatment. In addition to retaining all of the classical properties of anti-EGFR mAbs, including inhibition of ligand binding, inhibition of EGFR phosphorylation, inhibition of downstream signaling, and induction of ADCC, binding of the Sym004 mAbs to EGFR leads to highly efficient receptor internalization and degradation, which in turn leads to profound inhibition of cancer cell growth (Pedersen et al., supra and Koefoed et al., MAbs 3:1-12 (2011)). This novel synergistic mechanism of EGFR elimination results in more effective blockade of EGFR signaling pathways and higher antitumor activity than that observed with single mAbs (Pedersen et al., supra, and lida et al., supra). Sym004 also induces complement-dependent cytotoxicity (CDC), an additional mechanism not observed with single mAbs that may lead to tumor cell cytotoxicity (Koefoed et al., supra). These novel properties have been documented to result in enhanced anti-tumor activity in a variety of cancer models mimicking clinical resistance to cetuximab (Dienstmann et al., Cancer Discov. 5:598-609 (2015); Pedersen et al., supra; and lida et al., supra). The preclinical data were confirmed and extended by documentation of clinical activity in mCRC patients with acquired resistance to anti-EGFR mAbs in the Sym004 Phase 1/2 study (Dienstmann et al., supra).

In a recent Phase 1 study, Sym004 was shown to be well tolerated at doses up to 12 mg/kg weekly, with grade 3 skin toxicities and hypomagnesemia as mechanism-based dose-limiting toxicities. Notably, Sym004 showed early signs of clinical activity in an expansion cohort of anti-EGFR antibody pre-treated mCRC patients. Tumor shrinkage was observed in 44% of patients (17 of 39) and 13% (5 patients) achieved partial responses (Dienstmann et al., supra).

The present invention uses compositions comprising at least a first anti-human EGFR antibody. In some embodiments, the compositions further comprise a second anti-human EGFR antibody that binds to an epitope of EGFR distinct from that bound by said first antibody. In certain embodiments, the first and second antibodies bind to distinct epitopes on the extracellular domain of EGFR (e.g., in domain III of the extracellular domain). The term “anti-EGFR antibody composition” refers to a composition comprising at least one anti-EGFR antibody or antigen-binding portion thereof. In some embodiments, the composition comprises two anti-EGFR antibodies or antigen-binding portions thereof. In some embodiments, the composition comprises three anti-EGFR antibodies or antigen-binding portions thereof.

In one embodiment, the antibody composition comprises a first anti-EGFR antibody or an antigen-binding portion thereof and a second anti-EGFR antibody or an antigen-binding portion thereof, wherein the first anti-EGFR antibody is selected from the group consisting of:
an anti-EGFR antibody or an antigen-binding portion thereof that competes for binding to human EGFR with an antibody having a heavy chain comprising the amino acid sequence of SEQ ID NO: 1 and a light chain comprising the amino acid sequence of SEQ ID NO: 2;
an anti-EGFR antibody or an antigen-binding portion thereof that binds to the same epitope of human EGFR as an antibody having a heavy chain comprising the amino acid sequence of SEQ ID NO: 1 and a light chain comprising the amino acid sequence of SEQ ID NO: 2;
an anti-EGFR antibody or an antigen-binding portion thereof having an H-CDR1, H-CDR2, and H-CDR3 comprising the amino acid sequences of SEQ ID NOs: 5, 6, and 7, respectively;
an anti-EGFR antibody or an antigen-binding portion thereof having an L-CDR1, L-CDR2, and L-CDR3 comprising the amino acid sequences of SEQ ID NOs: 8, 9, and 10, respectively;
an anti-EGFR antibody or an antigen-binding portion thereof having an H-CDR1, H-CDR2, and H-CDR3 comprising the amino acid sequences of SEQ ID NOs: 5, 6, and 7, respectively, and an L-CDR1, L-CDR2, and L-CDR3 comprising the amino acid sequences of SEQ ID NOs: 8, 9, and 10, respectively;
an anti-EGFR antibody or an antigen-binding portion thereof having a heavy chain comprising the amino acid sequence of SEQ ID NO: 1;
an anti-EGFR antibody or an antigen-binding portion thereof having a light chain comprising the amino acid sequence of SEQ ID NO: 2;
an anti-EGFR antibody or an antigen-binding portion thereof having a heavy chain comprising the amino acid sequence of SEQ ID NO: 1 and a light chain comprising the amino acid sequence of SEQ ID NO: 2;
an anti-EGFR antibody or an antigen-binding portion thereof having a heavy chain variable domain at least 90%, 91%, 92%, 93%, 94%, 95%, 96%, 97%, 98%, or 99% identical to the amino acid sequence of SEQ ID NO: 1 and a light chain variable domain at least 90%, 91%, 92%, 93%, 94%, 95%, 96%, 97%, 98%, or 99% identical to the amino acid sequence of SEQ ID NO: 2; and
an anti-EGFR antibody or an antigen-binding portion thereof having a heavy chain comprising the amino acid sequence of SEQ ID NO: 26 and a light chain comprising the amino acid sequence of SEQ ID NO: 24;
   and wherein the second anti-EGFR antibody is selected from the group consisting of:
an anti-EGFR antibody or an antigen-binding portion thereof that competes for binding to human EGFR with an antibody having a heavy chain comprising the amino acid sequence of SEQ ID NO: 3 and a light chain comprising the amino acid sequence of SEQ ID NO: 4;
an anti-EGFR antibody or an antigen-binding portion thereof that binds to the same epitope of human EGFR as an antibody having a heavy chain comprising the amino acid sequence of SEQ ID NO: 3 and a light chain comprising the amino acid sequence of SEQ ID NO: 4;
an anti-EGFR antibody or an antigen-binding portion thereof having an H-CDR1, H-CDR2, and H-CDR3 comprising the amino acid sequences of SEQ ID NOs: 11, 12, and 13, respectively;
an anti-EGFR antibody or an antigen-binding portion thereof having an L-CDR1, L-CDR2, and L-CDR3 comprising the amino acid sequences of SEQ ID NOs: 14, 15, and 16, respectively;
an anti-EGFR antibody or an antigen-binding portion thereof having an H-CDR1, H-CDR2, and H-CDR3 comprising the amino acid sequences of SEQ ID NOs: 11, 12, and 13, respectively, and an L-CDR1, L-CDR2, and L-CDR3 comprising the amino acid sequences of SEQ ID NOs: 14, 15 and 16, respectively;
an anti-EGFR antibody or an antigen-binding portion thereof having a heavy chain comprising the amino acid sequence of SEQ ID NO: 3;
an anti-EGFR antibody or an antigen-binding portion thereof having a light chain comprising the amino acid sequence of SEQ ID NO: 4;
an anti-EGFR antibody or an antigen-binding portion thereof having a heavy chain comprising the amino acid sequence of SEQ ID NO: 3 and a light chain comprising the amino acid sequence of SEQ ID NO: 4;
an anti-EGFR antibody or an antigen-binding portion thereof having a heavy chain variable domain at least 90%, 91%, 92%, 93%, 94%, 95%, 96%, 97%, 98%, or 99% identical to the amino acid sequence of SEQ ID NO: 3 and a light chain variable domain at least 90%, 91%, 92%, 93%, 94%, 95%, 96%, 97%, 98%, or 99% identical to the amino acid sequence of SEQ ID NO: 4; and
an anti-EGFR antibody or an antigen-binding portion thereof having a heavy chain comprising the amino acid sequence of SEQ ID NO: 27 and a light chain comprising the amino acid sequence of SEQ ID NO: 25.
   Any combination of the above first and second anti-EGFR antibodies is contemplated.

In one embodiment, the antibody composition comprises: an anti-EGFR antibody or an antigen-binding portion thereof that competes for binding to human EGFR with an antibody having a heavy chain comprising the amino acid sequence of SEQ ID NO: 1 and a light chain comprising the amino acid sequence of SEQ ID NO: 2; and

an anti-EGFR antibody or an antigen-binding portion thereof that competes for binding to human EGFR with an antibody having a heavy chain comprising the amino acid sequence of SEQ ID NO: 3 and a light chain comprising the amino acid sequence of SEQ ID NO: 4.

In one embodiment, the antibody composition comprises: an anti-EGFR antibody or an antigen-binding portion thereof that binds to the same epitope of human EGFR as an antibody having a heavy chain comprising the amino acid sequence of SEQ ID NO: 1 and a light chain comprising the amino acid sequence of SEQ ID NO: 2; and

an anti-EGFR antibody or an antigen-binding portion thereof that binds to the same epitope of human EGFR as an antibody having a heavy chain comprising the amino acid sequence of SEQ ID NO: 3 and a light chain comprising the amino acid sequence of SEQ ID NO: 4.

In one embodiment, the antibody composition comprises: an anti-EGFR antibody or an antigen-binding portion thereof having an H-CDR1, H-CDR2, and H-CDR3 comprising the amino acid sequences of SEQ ID NOs: 5, 6, and 7, respectively; and

an anti-EGFR antibody or an antigen-binding portion thereof having an H-CDR1, H-CDR2, and H-CDR3 comprising the amino acid sequences of SEQ ID NOs: 11, 12, and 13, respectively.

In one embodiment, the antibody composition comprises:

an anti-EGFR antibody or an antigen-binding portion thereof having an L-CDR1, L-CDR2, and L-CDR3 comprising the amino acid sequences of SEQ ID NOs: 8, 9, and 10, respectively; and
an anti-EGFR antibody or an antigen-binding portion thereof having an L-CDR1, L-CDR2, and L-CDR3 comprising the amino acid sequences of SEQ ID NOs: 14, 15, and 16, respectively.

In one embodiment, the antibody composition comprises:

an anti-EGFR antibody or an antigen-binding portion thereof having an H-CDR1, H-CDR2, and H-CDR3 comprising the amino acid sequences of SEQ ID NOs: 5, 6, and 7, respectively, and an L-CDR1, L-CDR2, and L-CDR3 comprising the amino acid sequences of SEQ ID NOs: 8, 9, and 10, respectively; and
an anti-EGFR antibody or an antigen-binding portion thereof having an H-CDR1, H-CDR2, and H-CDR3 comprising the amino acid sequences of SEQ ID NOs: 11, 12, and 13, respectively, and an L-CDR1, L-CDR2, and L-CDR3 comprising the amino acid sequences of SEQ ID NOs: 14, 15, and 16, respectively.

In one embodiment, the antibody composition comprises: an anti-EGFR antibody or an antigen-binding portion thereof having a heavy chain comprising the amino acid sequence of SEQ ID NO: 1 and a light chain comprising the amino acid sequence of SEQ ID NO: 2; and an anti-EGFR antibody or an antigen-binding portion thereof having a heavy chain comprising the amino acid sequence of SEQ ID NO: 3 and a light chain comprising the amino acid sequence of SEQ ID NO: 4.

In one embodiment, the antibody composition comprises: an anti-EGFR antibody or an antigen-binding portion thereof having a heavy chain comprising the amino acid sequence of SEQ ID NO: 26 and a light chain comprising the amino acid sequence of SEQ ID NO: 24; and

an anti-EGFR antibody or an antigen-binding portion thereof having a heavy chain comprising the amino acid sequence of SEQ ID NO: 27 and a light chain comprising the amino acid sequence of SEQ ID NO: 25.

In one embodiment, the antibody composition comprises: an anti-EGFR antibody or an antigen-binding portion thereof having a heavy chain variable domain at least 90%, 91%, 92%, 93%, 94%, 95%, 96%, 97%, 98%, or 99% identical to the amino acid sequence of SEQ ID NO: 1 and a light chain variable domain at least 90%, 91%, 92%, 93%, 94%, 95%, 96%, 97%, 98%, or 99% identical to the amino acid sequence of SEQ ID NO: 2; and

an anti-EGFR antibody or an antigen-binding portion thereof having a heavy chain variable domain at least 90%, 91%, 92%, 93%, 94%, 95%, 96%, 97%, 98%, or 99% identical to the amino acid sequence of SEQ ID NO: 3 and a light chain variable domain at least 90%, 91%, 92%, 93%, 94%, 95%, 96%, 97%, 98%, or 99% identical to the amino acid sequence of SEQ ID NO: 4.
Any combination of the above identity percentages of the first and second antibodies is contemplated.

The ratio of the first antibody relative to the second antibody, or of the second antibody to the first antibody, may be between 5 and 95%, such as between 10 and 90%, between 20 and 80%, between 30 and 70%, between 40 and 60%, between 45 and 55%, or approximately 50% (i.e., a 1:1 ratio).

In some embodiments, the antibody composition comprises:

a first anti-EGFR antibody selected from the group consisting of:
an anti-EGFR antibody or an antigen-binding portion thereof that competes for binding to human EGFR with an antibody having a heavy chain comprising the amino acid sequence of SEQ ID NO: 3 and a light chain comprising the amino acid sequence of SEQ ID NO: 4;
an anti-EGFR antibody or an antigen-binding portion thereof that binds to the same epitope of human EGFR as an antibody having a heavy chain comprising the amino acid sequence of SEQ ID NO: 3 and a light chain comprising the amino acid sequence of SEQ ID NO: 4;
an anti-EGFR antibody or an antigen-binding portion thereof having an H-CDR1, H-CDR2, and H-CDR3 comprising the amino acid sequences of SEQ ID NOs: 11, 12, and 13, respectively;
an anti-EGFR antibody or an antigen-binding portion thereof having an L-CDR1, L-CDR2, and L-CDR3 comprising the amino acid sequences of SEQ ID NOs: 14, 15, and 16, respectively;
an anti-EGFR antibody or an antigen-binding portion thereof having an H-CDR1, H-CDR2, and H-CDR3 comprising the amino acid sequences of SEQ ID NOs: 11, 12, and 13, respectively, and an L-CDR1, L-CDR2, and L-CDR3 comprising the amino acid sequences of SEQ ID NOs: 14, 15 and 16, respectively;
an anti-EGFR antibody or an antigen-binding portion thereof having a heavy chain comprising the amino acid sequence of SEQ ID NO: 3;
an anti-EGFR antibody or an antigen-binding portion thereof having a light chain comprising the amino acid sequence of SEQ ID NO: 4;
an anti-EGFR antibody or an antigen-binding portion thereof having a heavy chain comprising the amino acid sequence of SEQ ID NO: 3 and a light chain comprising the amino acid sequence of SEQ ID NO: 4;
an anti-EGFR antibody or an antigen-binding portion thereof having a heavy chain variable domain at least 90%, 91%, 92%, 93%, 94%, 95%, 96%, 97%, 98%, or 99% identical to the amino acid sequence of SEQ ID NO: 3 and a light chain variable domain at least 90%, 91%, 92%, 93%, 94%, 95%, 96%, 97%, 98%, or 99% identical to the amino acid sequence of SEQ ID NO: 4; and
an anti-EGFR antibody or an antigen-binding portion thereof having a heavy chain comprising the amino acid sequence of SEQ ID NO: 27 and a light chain comprising the amino acid sequence of SEQ ID NO: 25; and
   a second anti-EGFR antibody selected from the group consisting:
an anti-EGFR antibody or an antigen-binding portion thereof that competes for binding to human EGFR with cetuximab or panitumumab;
an anti-EGFR antibody or an antigen-binding portion thereof that binds to the same epitope of human EGFR as cetuximab or panitumumab;
an anti-EGFR antibody or an antigen-binding portion thereof having the H-CDR1, H-CDR2, and H-CDR3 amino acid sequences of cetuximab or panitumumab;
an anti-EGFR antibody or an antigen-binding portion thereof having the L-CDR1, L-CDR2, and L-CDR3 amino acid sequences of cetuximab or panitumumab;
an anti-EGFR antibody or an antigen-binding portion thereof having the H-CDR1, H-CDR2, and H-CDR3 and L-CDR1, L-CDR2, and L-CDR3 amino acid sequences of cetuximab or panitumumab;
an anti-EGFR antibody or an antigen-binding portion thereof having the heavy chain variable amino acid sequence of cetuximab or panitumumab;
an anti-EGFR antibody or an antigen-binding portion thereof having the light chain variable domain amino acid sequence of cetuximab or panitumumab;
an anti-EGFR antibody or an antigen-binding portion thereof having the heavy and light chain variable domain amino acid sequences of cetuximab or panitumumab;
an anti-EGFR antibody or an antigen-binding portion thereof having a heavy chain variable domain at least 90%, 91%, 92%, 93%, 94%, 95%, 96%, 97%, 98%, or 99% identical to the heavy chain variable domain amino acid sequence, and a light chain variable domain at least 90%, 91%, 92%, 93%, 94%, 95%, 96%, 97%, 98%, or 99% identical to the light chain variable domain amino acid sequence, of cetuximab or panitumumab; and
an anti-EGFR antibody or an antigen-binding portion thereof having the heavy and light chain amino acid sequences of cetuximab or panitumumab.
   Any combination of the above first and second anti-EGFR antibodies is contemplated.

The ratio of the first antibody relative to the second antibody, or of the second antibody to the first antibody, may be between 5 and 95%, such as between 10 and 90%, between 20 and 80%, between 30 and 70%, between 40 and 60%, between 45 and 55%, or approximately 50% (i.e., a 1:1 ratio).

In one embodiment, the antibody composition comprises a first anti-EGFR antibody or an antigen-binding portion thereof, a second anti-EGFR antibody or an antigen-binding portion thereof, and a third anti-EGFR antibody or an antigen-binding portion thereof, wherein the first anti-EGFR antibody is selected from the group consisting of:
an anti-EGFR antibody or an antigen-binding portion thereof that competes for binding to human EGFR with an antibody having a heavy chain comprising the amino acid sequence of SEQ ID NO: 18 and a light chain comprising the amino acid sequence of SEQ ID NO: 17;
an anti-EGFR antibody or an antigen-binding portion thereof that binds to the same epitope of human EGFR as an antibody having a heavy chain comprising the amino acid sequence of SEQ ID NO: 18 and a light chain comprising the amino acid sequence of SEQ ID NO: 17;
an anti-EGFR antibody or an antigen-binding portion thereof having an H-CDR1, H-CDR2, and H-CDR3 comprising the H-CDR1, H-CDR2, and H-CDR3 amino acid sequences shown in SEQ ID NO: 18;
an anti-EGFR antibody or an antigen-binding portion thereof having an L-CDR1, L-CDR2, and L-CDR3 comprising the L-CDR1, L-CDR2, and L-CDR3 amino acid sequences shown in SEQ ID NO: 17;
an anti-EGFR antibody or an antigen-binding portion thereof having an H-CDR1, H-CDR2, and H-CDR3 comprising the H-CDR1, H-CDR2, and H-CDR3 amino acid sequences shown in SEQ ID NO: 18, and an L-CDR1, L-CDR2, and L-CDR3 comprising the L-CDR1, L-CDR2, and L-CDR3 amino acid sequences shown in SEQ ID NO: 17;
an anti-EGFR antibody or an antigen-binding portion thereof having a heavy chain comprising the amino acid sequence of SEQ ID NO: 18;
an anti-EGFR antibody or an antigen-binding portion thereof having a light chain comprising the amino acid sequence of SEQ ID NO: 17;
an anti-EGFR antibody or an antigen-binding portion thereof having a heavy chain comprising the amino acid sequence of SEQ ID NO: 18 and a light chain comprising the amino acid sequence of SEQ ID NO: 17; and
an anti-EGFR antibody or an antigen-binding portion thereof having a heavy chain variable domain at least 90%, 91%, 92%, 93%, 94%, 95%, 96%, 97%, 98%, or 99% identical to the amino acid sequence of SEQ ID NO: 18 and a light chain variable domain at least 90%, 91%, 92%, 93%, 94%, 95%, 96%, 97%, 98%, or 99% identical to the amino acid sequence of SEQ ID NO: 17;
   wherein the second anti-EGFR antibody is selected from the group consisting of:
an anti-EGFR antibody or an antigen-binding portion thereof that competes for binding to human EGFR with an antibody having a heavy chain comprising the amino acid sequence of SEQ ID NO: 20 and a light chain comprising the amino acid sequence of SEQ ID NO: 19;
an anti-EGFR antibody or an antigen-binding portion thereof that binds to the same epitope of human EGFR as an antibody having a heavy chain comprising the amino acid sequence of SEQ ID NO: 20 and a light chain comprising the amino acid sequence of SEQ ID NO: 19;
an anti-EGFR antibody or an antigen-binding portion thereof having an H-CDR1, H-CDR2, and H-CDR3 comprising the H-CDR1, H-CDR2, and H-CDR3 amino acid sequences shown in SEQ ID NO: 20;
an anti-EGFR antibody or an antigen-binding portion thereof having an L-CDR1, L-CDR2, and L-CDR3 comprising the L-CDR1, L-CDR2, and L-CDR3 amino acid sequences shown in SEQ ID NO: 19;
an anti-EGFR antibody or an antigen-binding portion thereof having an H-CDR1, H-CDR2, and H-CDR3 comprising the H-CDR1, H-CDR2, and H-CDR3 amino acid sequences shown in SEQ ID NO: 20, and an L-CDR1, L-CDR2, and L-CDR3 comprising the L-CDR1, L-CDR2, and L-CDR3 amino acid sequences shown in SEQ ID NO: 19;
an anti-EGFR antibody or an antigen-binding portion thereof having a heavy chain comprising the amino acid sequence of SEQ ID NO: 20;
an anti-EGFR antibody or an antigen-binding portion thereof having a light chain comprising the amino acid sequence of SEQ ID NO: 19;
an anti-EGFR antibody or an antigen-binding portion thereof having a heavy chain comprising the amino acid sequence of SEQ ID NO: 20 and a light chain comprising the amino acid sequence of SEQ ID NO: 19; and
an anti-EGFR antibody or an antigen-binding portion thereof having a heavy chain variable domain at least 90%, 91%, 92%, 93%, 94%, 95%, 96%, 97%, 98%, or 99% identical to the amino acid sequence of SEQ ID NO: 20 and a light chain variable domain at least 90%, 91%, 92%, 93%, 94%, 95%, 96%, 97%, 98%, or 99% identical to the amino acid sequence of SEQ ID NO: 19;
   and wherein the third anti-EGFR antibody is selected from the group consisting of:
an anti-EGFR antibody or an antigen-binding portion thereof that competes for binding to human EGFR with an antibody having a heavy chain comprising the amino acid sequence of SEQ ID NO: 22 and a light chain comprising the amino acid sequence of SEQ ID NO: 21;
an anti-EGFR antibody or an antigen-binding portion thereof that binds to the same epitope of human EGFR as an antibody having a heavy chain comprising the amino acid sequence of SEQ ID NO: 22 and a light chain comprising the amino acid sequence of SEQ ID NO: 21;
an anti-EGFR antibody or an antigen-binding portion thereof having an H-CDR1, H-CDR2, and H-CDR3 comprising the H-CDR1, H-CDR2, and H-CDR3 amino acid sequences shown in SEQ ID NO: 22;
an anti-EGFR antibody or an antigen-binding portion thereof having an L-CDR1, L-CDR2, and L-CDR3 comprising the L-CDR1, L-CDR2, and L-CDR3 amino acid sequences shown in SEQ ID NO: 21;
an anti-EGFR antibody or an antigen-binding portion thereof having an H-CDR1, H-CDR2, and H-CDR3 comprising the H-CDR1, H-CDR2, and H-CDR3 amino acid sequences shown in SEQ ID NO: 22, and an L-CDR1, L-CDR2, and L-CDR3 comprising the L-CDR1, L-CDR2, and L-CDR3 amino acid sequences shown in SEQ ID NO: 21;
an anti-EGFR antibody or an antigen-binding portion thereof having a heavy chain comprising the amino acid sequence of SEQ ID NO: 22;
an anti-EGFR antibody or an antigen-binding portion thereof having a light chain comprising the amino acid sequence of SEQ ID NO: 21;
an anti-EGFR antibody or an antigen-binding portion thereof having a heavy chain comprising the amino acid sequence of SEQ ID NO: 22 and a light chain comprising the amino acid sequence of SEQ ID NO: 21; and
an anti-EGFR antibody or an antigen-binding portion thereof having a heavy chain variable domain at least 90%, 91%, 92%, 93%, 94%, 95%, 96%, 97%, 98%, or 99% identical to the amino acid sequence of SEQ ID NO: 22 and a light chain variable domain at least 90%, 91%, 92%, 93%, 94%, 95%, 96%, 97%, 98%, or 99% identical to the amino acid sequence of SEQ ID NO: 21.

In certain embodiments, the ratio of the first, second, and third antibodies is 2:2:1.

In certain embodiments, the first, second, and third antibodies are P1X, P2X, and P3X as described in U.S. Patent Publication US20120308576, hereby incorporated by reference.

In one embodiment, the antibody composition comprises a first anti-EGFR antibody or an antigen-binding portion thereof and a second anti-EGFR antibody or an antigen-binding portion thereof, wherein the first and second anti-EGFR antibodies are selected from the chimeric anti-EGFR antibodies described in U.S. Pat. No. 7,887,805. In some embodiments, the first and second anti-EGFR antibodies bind to different epitopes of EGFR. In certain embodiments, the first and second anti-EGFR antibodies compete for binding to EGFR with, bind to the same epitope of EGFR as, have heavy and light chain variable domains that are at least 90%, 92%, 95%, 96%, 97%, 98%, or 99% identical in amino acid sequence to the heavy and light chain variable domains of, comprise the six CDRs of, or comprise the heavy and light chain variable domains of, a first and second antibody selected from the chimeric anti-EGFR antibodies described in U.S. Pat. No. 7,887,805. In particular embodiments, the first antibody is selected from the group consisting of antibodies 992, 1209, 1204, 996, 1033, and 1220 and the second antibody is selected from the group consisting of antibodies 1024, 1031, 1036, 1042, 984, 1210, 1217, 1221, and 1218.

Any of the anti-EGFR antibodies described herein can be an IgG, an IgM, an IgE, an IgA, or an IgD molecule, but is typically of the IgG isotype, e.g., of IgG subclass IgG1, IgG2a, or IgG2b. In a particular embodiment, the antibody is an IgG1. In another embodiment, the antibody is an IgG2.

In some embodiments, an anti-EGFR antibody described herein may comprise at least one mutation in the Fc region. A number of different Fc mutations are known, where these mutations provide altered effector function. For example, in many cases it will be desirable to reduce or eliminate effector function, e.g., where ligand/receptor interactions are undesired or in the case of antibody-drug conjugates.

In some embodiments, an anti-EGFR antibody described herein comprises at least one mutation in the Fc region that reduces effector function. Fc region amino acid positions that may be advantageous to mutate in order to reduce effector function include one or more of positions 228, 233, 234 and 235, where amino acid positions are numbered according to the IMGT® numbering scheme.

In some embodiments, one or both of the amino acid residues at positions 234 and 235 may be mutated, for example, from Leu to Ala (L234A/L235A). These mutations reduce effector function of the Fc region of IgG1 antibodies. Additionally or alternatively, the amino acid residue at position 228 may be mutated, for example to Pro. In some embodiments, the amino acid residue at position 233 may be mutated, e.g., to Pro, the amino acid residue at position 234 may be mutated, e.g., to Val, and/or the amino acid residue at position 235 may be mutated, e.g., to Ala. The amino acid positions are numbered according to the IMGT® numbering scheme.

In another embodiment, where the antibody is of the IgG4 subclass, it may comprise the mutation S228P, i.e., having a proline in position 228, where the amino acid position is numbered according to the IMGT® numbering scheme. This mutation is known to reduce undesired Fab arm exchange.

In certain embodiments, an antibody or antigen-binding portion thereof as described herein may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov et al., Human Antibodies and Hybridomas 6:93-101 (1995)) and use of a cysteine residue, a marker peptide and a C-terminal polyhistidine tag to make bivalent and biotinylated scFv molecules (Kipriyanov et al., Mol. Immunol. 31:1047-1058 (1994)). Other examples include where one or more CDRs from an antibody are incorporated into a molecule either covalently or noncovalently to make it an immunoadhesin that specifically binds to an antigen of interest. In such embodiments, the CDR(s) may be incorporated as part of a larger polypeptide chain, may be covalently linked to another polypeptide chain, or may be incorporated noncovalently.

In another embodiment, a fusion antibody or immunoadhesin may be made that comprises all or a portion of an anti-EGFR antibody described herein linked to another polypeptide. In certain embodiments, only the variable domains of the anti-EGFR antibody are linked to the polypeptide. In certain embodiments, the VH domain of an anti-EGFR antibody is linked to a first polypeptide, while the VL domain of an anti-EGFR antibody is linked to a second polypeptide that associates with the first polypeptide in a manner such that the VH and VL domains can interact with one another to form an antigen-binding site. In another preferred embodiment, the VH domain is separated from the VL domain by a linker such that the VH and VL domains can interact with one another (e.g., single-chain antibodies). The VH-linker-VL antibody is then linked to the polypeptide of interest. In addition, fusion antibodies can be created in which two (or more) single-chain antibodies are linked to one another. This is useful if one wants to create a divalent or polyvalent antibody on a single polypeptide chain, or if one wants to create a bispecific antibody.

To create a single chain antibody (scFv), the VH- and VL-encoding DNA fragments are operatively linked to another fragment encoding a flexible linker, e.g., encoding the amino acid sequence (Gly4-Ser)3 (SEQ ID NO: 32), such that the VH and VL sequences can be expressed as a contiguous single-chain protein, with the VL and VH domains joined by the flexible linker. See, e.g., Bird et al., Science 242:423-426 (1988); Huston et al., Proc. Natl. Acad. Sci. USA 85:5879-5883 (1988); and McCafferty et al., Nature 348:552-554 (1990). The single chain antibody may be monovalent, if only a single VH and VL are used; bivalent, if two VH and VL are used; or polyvalent, if more than two VH and VL are used. Bispecific or polyvalent antibodies may be generated that bind specifically to human EGFR and to another molecule, for instance.

In other embodiments, other modified antibodies may be prepared using anti-EGFR antibody-encoding nucleic acid molecules. For instance, “kappa bodies” (III et al., Protein Eng. 10:949-57 (1997)), “minibodies” (Martin et al., EMBO J. 13:5303-9 (1994)), “diabodies” (Holliger et al., Proc. Natl. Acad. Sci. USA 90:6444-6448 (1993)), or “Janusins” (Traunecker et al., EMBO J. 10:3655-3659 (1991) and Traunecker et al., Int. J. Cancer (Suppl.) 7:51-52 (1992)) may be prepared using standard molecular biological techniques following the teachings of the specification.

An anti-EGFR antibody or antigen-binding portion described herein can be derivatized or linked to another molecule (e.g., another peptide or protein). In general, the antibodies or portions thereof are derivatized such that EGFR binding is not affected adversely by the derivatization or labeling. Accordingly, the antibodies and antibody portions that may be used in the therapies of the invention are intended to include both intact and modified forms of the human anti-EGFR antibodies described herein. For example, an antibody or antibody portion described herein can be functionally linked (by chemical coupling, genetic fusion, noncovalent association or otherwise) to one or more other molecular entities, such as another antibody (e.g., a bispecific antibody or a diabody), a detection agent, a pharmaceutical agent, and/or a protein or peptide that can mediate association of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available, e.g., from Pierce Chemical Company, Rockford, Ill.

An anti-EGFR antibody can also be derivatized with a chemical group such as polyethylene glycol (PEG), a methyl or ethyl group, or a carbohydrate group. These groups may be useful to improve the biological characteristics of the antibody, e.g., to increase serum half-life.

An antibody described herein may also be labeled. As used herein, the terms “label” or “labeled” refer to incorporation of another molecule in the antibody. In one embodiment, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). In another embodiment, the label or marker can be therapeutic, e.g., a drug conjugate or toxin. Various methods of labeling polypeptides and glycoproteins are known in the art and may be used. Examples of labels for polypeptides include, but are not limited to, the following: radioisotopes or radionuclides (e.g., 3H, 14C, 15N, 35S, 90Y, 99Tc, 111In, 125I, 131I), fluorescent labels (e.g., FITC, rhodamine, lanthanide phosphors), enzymatic labels (e.g., horseradish peroxidase, β-galactosidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), magnetic agents, such as gadolinium chelates, toxins such as pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. In some embodiments, labels are attached by spacer arms of various lengths to reduce potential steric hindrance.

In certain embodiments, the antibodies described herein may be present in a neutral form (including zwitter ionic forms) or as a positively or negatively-charged species. In some embodiments, the antibodies may be complexed with a counterion to form a pharmaceutically acceptable salt.

The term “pharmaceutically acceptable salt” refers to a complex comprising one or more antibodies and one or more counterions, wherein the counterions are derived from pharmaceutically acceptable inorganic and organic acids and bases.

### Bispecific and Trispecific Binding Molecules

In a further aspect, the binding specificities of any two individual antibodies disclosed herein may be combined in one bispecific binding molecule, or the binding specificities of any three individual antibodies disclosed herein may be combined in one trispecific binding molecule. For example, a bispecific binding molecule may have the binding specificities of anti-EGFR antibodies 992 and 1024. In some embodiments, the bispecific binding molecule may have the binding specificities of: an anti-EGFR antibody or an antigen-binding portion thereof having a heavy chain variable domain comprising the amino acid sequence of SEQ ID NO: 1 and a light chain variable domain comprising the amino acid sequence of SEQ ID NO: 2; and an anti-EGFR antibody or an antigen-binding portion thereof having a heavy chain variable domain comprising the amino acid sequence of SEQ ID NO: 3 and a light chain variable domain comprising the amino acid sequence of SEQ ID NO: 4.

In some embodiments, the bispecific binding molecule may comprise the CDR amino acid sequences of SEQ ID NOs: 5-10 and the CDR amino acid sequences of SEQ ID NOs: 11-16. In some embodiments, the bispecific binding molecule may comprise the heavy and light chain variable domain amino acid sequences of SEQ ID NOs: 1 and 2 and the heavy and light chain variable domain amino acid sequences of SEQ ID NOs: 3 and 4.

The bispecific binding molecule may be a dual variable domain antibody, i.e., wherein the two arms of the antibody comprise two different variable domains, or may be in the form of an antibody fragment such as a bispecific Fab fragment or a bispecific scFv.

In some embodiments, the trispecific binding molecule may have the binding specificities of:
an anti-EGFR antibody or an antigen-binding portion thereof having a heavy chain variable domain comprising the amino acid sequence of SEQ ID NO: 18 and a light chain variable domain comprising the amino acid sequence of SEQ ID NO: 17;
an anti-EGFR antibody or an antigen-binding portion thereof having a heavy chain variable domain comprising the amino acid sequence of SEQ ID NO: 20 and a light chain variable domain comprising the amino acid sequence of SEQ ID NO: 19; and
an anti-EGFR antibody or an antigen-binding portion thereof having a heavy chain variable domain comprising the amino acid sequence of SEQ ID NO: 22 and a light chain variable domain comprising the amino acid sequence of SEQ ID NO: 21.

In some embodiments, the trispecific binding molecule may comprise the six CDRs in the heavy and light chain variable domain amino acid sequences of SEQ ID NOs: 18 and 17, the six CDRs in the heavy and light chain variable domain amino acid sequences of SEQ ID NOs: 20 and 19, and the six CDRs in the heavy and light chain variable domain amino acid sequences of SEQ ID NOs: 22 and 21. In some embodiments, the trispecific binding molecule may comprise the heavy and light chain variable domain amino acid sequences of SEQ ID NOs: 18 and 17, the heavy and light chain variable domain amino acid sequences of SEQ ID NOs: 20 and 19, and the heavy and light chain variable domain amino acid sequences of SEQ ID NOs: 22 and 21.

The trispecific binding molecule may be in the form of an antibody fragment such as a trispecific Fab fragment or a trispecific scFv.

### Pharmaceutical Compositions

Another aspect of the invention is a pharmaceutical composition comprising as active ingredients (e.g., as the sole active ingredients) one or more anti-EGFR antibody molecules or antigen-binding portions thereof, or anti-EGFR antibody compositions, described herein.

Generally, the antibodies, antigen-binding portions thereof, and compositions are suitable to be administered as a formulation in association with one or more pharmaceutically acceptable excipient(s), e.g., as described below.

The term “excipient” is used herein to describe any ingredient other than the compound(s) of the invention. The choice of excipient(s) will to a large extent depend on factors such as the particular mode of administration, the effect of the excipient on solubility and stability, and the nature of the dosage form. As used herein, “pharmaceutically acceptable excipient” includes any and all solvents, dispersion media, coatings, antibacterial and antifungal agents, isotonic and absorption delaying agents, and the like that are physiologically compatible. Some examples of pharmaceutically acceptable excipients are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody.

Pharmaceutical compositions described herein and methods for their preparation will be readily apparent to those skilled in the art. Such compositions and methods for their preparation may be found, for example, in Remington's Pharmaceutical Sciences, 19th Edition (Mack Publishing Company, 1995). Pharmaceutical compositions are preferably manufactured under GMP (good manufacturing practices) conditions.

A pharmaceutical composition described herein may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

Any method for administering peptides, proteins or antibodies accepted in the art may suitably be employed for the antibodies and antigen-binding portions described herein.

The pharmaceutical compositions described herein are typically suitable for parenteral administration. As used herein, “parenteral administration” of a pharmaceutical composition includes any route of administration characterized by physical breaching of a tissue of a subject and administration of the pharmaceutical composition through the breach in the tissue, thus generally resulting in the direct administration into the blood stream, into muscle, or into an internal organ. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal, intravenous, intraarterial, intrathecal, intraventricular, intraurethral, intracranial, intratumoral, and intrasynovial injection or infusions; and kidney dialytic infusion techniques. Regional perfusion is also contemplated. Particular embodiments include the intravenous and the subcutaneous routes.

Formulations of a pharmaceutical composition suitable for parenteral administration typically comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampoules or in multi-dose containers containing a preservative. Formulations for parenteral administration include, but are not limited to, suspensions, solutions, emulsions in oily or aqueous vehicles, pastes, and the like. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition. Parenteral formulations also include aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water. Exemplary parenteral administration forms include solutions or suspensions in sterile aqueous solutions, for example, aqueous propylene glycol or dextrose solutions. Such dosage forms can be suitably buffered, if desired. Other parentally-administrable formulations which are useful include those which comprise the active ingredient in microcrystalline form, or in a liposomal preparation. Formulations for parenteral administration may be formulated to be immediate and/or modified release. Modified release formulations include delayed-, sustained-, pulsed-, controlled-, targeted and programmed release.

For example, in one aspect, sterile injectable solutions can be prepared by incorporating an anti-EGFR antibody composition described herein in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin, and/or by using modified-release coatings (e.g., slow-release coatings).

### Therapeutic Uses of Antibodies and Compositions Described Herein

The anti-EGFR antibodies and antibody compositions described herein may be used for the treatment or amelioration of a disease in a mammal, in particular a human. In one aspect, the anti-EGFR antibodies and antibody compositions described herein are used in the treatment of a disorder that can be affected by EGFR activity. Typical EGFR-related diseases which can be treated, ameliorated, and/or prevented using the antibodies described herein include, but are not limited to, autoimmune diseases and cancers. For example, cancers which can be treated, ameliorated, and/or prevented include cancer of the bladder, breast, uterus/cervix, kidney, ovary, prostate, renal cell, pancreas, colon, rectum, stomach, squamous cell, lung (non-small cell), esophagus, head and neck, and skin. Autoimmune diseases which may be treated include, for example, psoriasis.

In some embodiments, the invention relates to a method for the treatment, amelioration, and/or prevention of glioblastoma, including glioblastoma multiforme; astrocytoma, including childhood astrocytoma; glioma; neuroblastoma; neuroendocrine tumors of the gastrointestinal tract; bronchoalveolar carcinoma; follicular dendritic cell sarcoma; salivary gland carcinoma; ameloblastoma; malignant peripheral nerve sheet tumor; endocrine pancreatic tumors; or testicular germ cell tumors, including seminoma, embryonal carcinoma, yolk sac tumor, teratoma and choriocarcinoma. Antibodies described herein are indicated in the treatment of certain solid tumours. Based upon a number of factors, including EGFR expression levels, among others, the following tumour types appear to present exemplary indications: breast, ovarian, colon, rectum, prostate, bladder, pancreas, head and neck, and non-small cell lung cancer. In certain embodiments, the invention relates to a method for the treatment of metastatic colorectal cancer.

In some embodiments, the invention relates to a method of treating a carcinoma or sarcoma. Carcinomas include, e.g., epithelial neoplasms, squamous cell neoplasms, squamous cell carcinoma, basal cell neoplasms, basal cell carcinoma, transitional cell papillomas and carcinomas, adenomas and adenocarcinomas, adenocarcinoma, linitis plastica, insulinoma, glucagonoma, gastrinoma, vipoma, cholangiocarcinoma, hepatocellular carcinoma, adenoid cystic carcinoma, carcinoid tumor of appendix, prolactinoma, oncocytoma, Hurthle cell adenoma, renal cell carcinoma, Grawitz tumor, multiple endocrine adenomas, endometrioid adenoma, adnexal and skin appendage neoplasms, mucoepidermoid neoplasms, cystic, mucinous and serous neoplasms, cystadenoma, pseudomyxoma peritonei, ductal, lobular and medullary neoplasms, acinar cell neoplasms, complex epithelial neoplasms, Warthin's tumor, thymoma, specialized gonadal neoplasms, sex cord-stromal tumor, thecoma, granulosa cell tumor, arrhenoblastoma, Sertoli-Leydig cell tumor, paragangliomas and Glomus tumors, pheochromocytoma, nevi and melanomas, melanocytic nevus, melanoma, nodular melanoma, dysplastic nevus, lentigo maligna melanoma, superficial spreading melanoma, and acral lentiginous melanoma.

“Treat”, “treating” and “treatment” refer to a method of alleviating or abrogating a biological disorder and/or at least one of its attendant symptoms. As used herein, to “alleviate” a disease, disorder or condition means reducing the severity and/or occurrence frequency of the symptoms of the disease, disorder, or condition. Further, references herein to “treatment” include references to curative, palliative and prophylactic treatment.

“Therapeutically effective amount” refers to the amount of the therapeutic agent being administered that will relieve to some extent one or more of the symptoms of the disorder being treated. A therapeutically effective amount of an anti-cancer therapeutic may, for example, result in tumor shrinkage, increased survival, elimination of cancer cells, decreased disease progression, reversal of metastasis, or other clinical endpoints desired by healthcare professionals.

The antibody compositions or antibodies or antigen-binding portions thereof described herein may be administered alone (e.g., as a combination of two or more antibodies or antigen-binding portions thereof, co-administered without other active agents) or in combination with one or more other drugs or antibodies (or as any combination thereof). The pharmaceutical compositions, methods and uses described herein thus also encompass embodiments of combinations (co-administration) with other active agents, as detailed below.

As used herein, the terms “co-administration,” “co-administered,” and “in combination with,” referring to a combination of the antibodies or antigen-binding portions thereof described herein, or referring to one or more antibody compositions or antibodies and antigen-binding portions thereof described herein with one or more other therapeutic agents, is intended to mean, and does refer to and include the following:
simultaneous administration of such combination of antibodies or antigen-binding portions, or such antibody composition/antibody/antigen-binding portion and therapeutic agent(s), to a patient in need of treatment, when such components are formulated together into a single dosage form which releases said components at substantially the same time to said patient,
substantially simultaneous administration of such combination of antibodies or antigen-binding portions, or such antibody composition/antibody/antigen-binding portion and therapeutic agent(s), to a patient in need of treatment, when such components are formulated apart from each other into separate dosage forms which are taken at substantially the same time by said patient, whereupon said components are released at substantially the same time to said patient,
sequential administration of such combination of antibodies or antigen-binding portions, or such antibody composition/antibody/antigen-binding portion and therapeutic agent(s), to a patient in need of treatment, when such components are formulated apart from each other into separate dosage forms which are taken at consecutive times by said patient with a significant time interval between each administration, whereupon said components are released at substantially different times to said patient; and
sequential administration of such combination of antibodies or antigen-binding portions, or such antibody composition/antibody/antigen-binding portion and therapeutic agent(s), to a patient in need of treatment, when such components are formulated together into a single dosage form which releases said components in a controlled manner whereupon they are concurrently, consecutively, and/or overlappingly released at the same and/or different times to said patient, where each part may be administered by either the same or a different route.

The antibody compositions and antibodies and antigen-binding portions thereof described herein may be administered without additional therapeutic treatments, i.e., as a stand-alone therapy (i.e., monotherapy). Alternatively, treatment with the antibody compositions and antibodies and antigen-binding portions thereof described herein may include at least one additional therapeutic treatment (combination therapy). In some embodiments, the antibody composition or antibody or antigen-binding portion thereof may be used in combination with another medication/drug for the treatment of cancer. The additional therapeutic treatment may comprise, e.g., a chemotherapeutic, anti-neoplastic, or anti-angiogenic agent, a different anti-cancer antibody, and/or radiation therapy.

In some embodiments, the additional therapeutic treatment is an agent capable of inducing terminal differentiation of cancer cells. The agent may, for example, be selected from the group consisting of retinoic acid, trans-retinoic acids, cis-retinoic acids, phenylbutyrate, nerve growth factor, dimethyl sulfoxide, active form vitamin D3, peroxisome proliferator-activated receptor gamma, 12-O-tetradecanoyl phorbol 13-acetate, hexamethylene-bis-acetamide, transforming growth factor-beta, butyric acid, cyclic AMP, and vesnarinone. In some embodiments, the compound is selected from the group consisting of retinoic acid, phenylbutyrate, all-trans-retinoic acid and active form vitamin D.

In some embodiments, the additional therapeutic treatment is a chemotherapeutic agent suitable for treatment of the particular cancer in question, for example, an agent selected from the group including, but not limited to, adriamycin, taxol, doxorubicin, topotecan, alkylating agents, e.g., platinum derivatives such as cisplatin, carboplatin and/or oxaliplatin; plant alkoids, e.g., paclitaxel, docetaxel and/or irinotecan; antitumor antibiotics, e.g., doxorubicin (adriamycin), daunorubicin, epirubicin, idarubicin mitoxantrone, dactinomycin, bleomycin, actinomycin, luteomycin, and/or mitomycin; topoisomerase inhibitors such as topotecan; and/or antimetabolites, e.g., fluorouracil and/or other fluoropyrimidines.

In some embodiments, the additional therapeutic treatment is a tyrosine kinase inhibitor selected from the group including, but not limited to, regorafenib (Stivarga), gefitinib (Iressa, ZD1839), erlobtinib (Tarceva, OSI-774), lapatinib, (Tykerb, GW572016), canertinib (CI-1033), pelitinib (EKB-569), and PKI-166.

In some embodiments, the additional therapeutic treatment is a small molecule inhibitor selected from the group including, but not limited to, sorafinib, sunitinib, temsirolimus, everolimus (RAD001), and cediranib (AZD217).

In some embodiments, the additional therapeutic treatment is another antibody therapeutic. Examples of these include, e.g., antibodies against HER2 (e.g., Herceptin HER3, and VEGF (e.g., Avastin®). Other anti-EGFR antibodies are also contemplated, wherein such anti-EGFR antibodies are neither the antibodies of the anti-EGFR antibody composition nor antibodies to which the patient has developed resistance.

In some embodiments, the additional therapeutic treatment is an agent known to stimulate cells of the immune system. Examples of such immune-stimulating agents include but are not limited to recombinant interleukins (e.g., IL-21 and IL-2)

In some embodiments, the additional therapeutic treatment is a MET inhibitor (e.g., an antibody or small molecule), a BRAF inhibitor (e.g., vemurafenib or dabrafenib), and/or a MEK inhibitor (e.g., trametinib or cobimetinib).

An anti-EGFR antibody composition described herein may also be used in combination with other anti-cancer therapies such as vaccines, cytokines, enzyme inhibitors and T cell therapies. In the case of a vaccine, it may e.g., be a protein, peptide or DNA vaccine containing one or more antigens which are relevant for the cancer being treated, or a vaccine comprising dendritic cells along with an antigen. Suitable cytokines include, for example, IL-2, IFN-gamma and GM-CSF. An example of a type of enzyme inhibitor that has anti-cancer activity is an indoleamine-2,3-dioxygenase (IDO) inhibitor, for example 1-methyl-D-tryptophan (1-D-MT). Adoptive T cell therapy refers to various immunotherapy techniques that involve expanding or engineering patients' own T cells to recognize and attack their tumors.

It is also contemplated that an anti-EGFR antibody composition described herein may be used in adjunctive therapy in connection with tyrosine kinase inhibitors.

In some embodiments, an anti-EGFR antibody composition described herein is used in a combination therapy together with chemotherapy, at least one tyrosine kinase inhibitor, at least one angiogenesis inhibitor, at least one hormone, at least one differentiation inducing agent, or any combination of the above. In certain embodiments, the angiogenesis inhibitor is bevacizumab. In certain embodiments, the anti-EGFR antibody composition is used in a combination therapy with, e.g., FOLFIRI, FOLFOX, RTx, TAS102, an inhibitor of PD1 and/or of another immune checkpoint protein, or any combination thereof.

It is understood that the antibody compositions and antibodies and antigen-binding portions thereof described herein may be used in a method of treatment as described herein, may be for use in a treatment as described herein, and/or may be for use in the manufacture of a medicament for a treatment as described herein. The invention also provides kits and articles of manufacture comprising the antibody compositions, antibodies, and antigen-binding portions thereof as described herein.

### Dose and Route of Administration

The antibody compositions described herein will be administered in an effective amount for treatment of the condition in question, i.e., at dosages and for periods of time necessary to achieve a desired result. A therapeutically effective amount may vary according to factors such as the particular condition being treated, the age, sex and weight of the patient, and whether the antibodies are being administered as a stand-alone treatment or in combination with one or more additional anti-cancer treatments.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the patients/subjects to be treated; each unit containing a predetermined quantity of active compound calculated to produce the desired therapeutic effect in association with the required pharmaceutical carrier. The specification for the dosage unit forms of the invention are generally dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, the skilled artisan would appreciate, based upon the disclosure provided herein, that the dose and dosing regimen are adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the embodied composition. Further, the dosage regimen with the compositions of this invention may be based on a variety of factors, including the type of disease, the age, weight, sex, medical condition of the patient, the severity of the condition, the route of administration, and the particular antibody employed. Thus, the dosage regimen can vary widely, but can be determined routinely using standard methods. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regimens are well-known in the relevant art and would be understood to be encompassed by the skilled artisan once provided the teachings disclosed herein.

Examples of suitable routes of administration are provided above.

It is contemplated that a suitable dose of an antibody composition described herein will be in the range of 0.1-100 mg/kg, such as about 0.5-50 mg/kg, e.g., about 1-20 mg/kg. The antibody composition may for example be administered at a dosage of at least 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, or 20 mg/kg. In some embodiments, the antibody composition may for example be administered at a dosage of up to at most 20 mg/kg, 15 mg/kg, 14 mg/kg, 13 mg/kg, 12 mg/kg, 11 mg/kg, 10 mg/kg, 9 mg/kg, 8 mg/kg, 7 mg/kg, 6 mg/kg, 5 mg/kg, 4 mg/kg, 3 mg/kg, 2 mg/kg, or 1 mg/kg. In some embodiments, the antibody composition may for example be administered at a dosage of at least 100 mg, 110 mg, 120 mg, 130 mg, 140 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, 1000 mg, 1250 mg, 1500 mg, 2000 mg, or 3000 mg. In some embodiments, the antibody composition may for example be administered at a dosage of up to at most 3000 mg, 2000 mg, 1500 mg, 1250 mg, 1000 mg, 950 mg, 900 mg, 850 mg, 800 mg, 750 mg, 700 mg, 650 mg, 600 mg, 550 mg, 500 mg, 450 mg, 400 mg, 350 mg, 300 mg, 250 mg, 200 mg, 150 mg, 140 mg, 130 mg, 120 mg, 110 mg, or 100 mg.

In certain embodiments, the antibody composition may be administered as one or more loading doses and subsequent doses at suitable intervals, wherein the loading dose(s) are different (e.g., higher) than the subsequent doses. The loading doses may be successively increasing, successively decreasing, or the same in amount. In one embodiment, the patient is given one loading dose of 9 mg/kg. The subsequent doses also may be successively increasing, successively decreasing, or the same. For example, after reaching a desired clinical endpoint (e.g., tumor reduction or cancer remission), a lower maintenance dose may be administered to the patient, to prevent cancer recurrence or minimize residual disease. In one embodiment, the subsequent doses are administered starting at 6 mg/kg, which may stay the same or may decrease after reaching a desired clinical endpoint.

In certain embodiments, the antibody composition is administered in doses starting at 12 mg/kg, which may stay the same or may decrease after reaching a desired clinical endpoint.

Administration will normally be repeated at suitable intervals, e.g., once every week, once every two weeks, once every three weeks, or once every four weeks, and for as long as deemed appropriate by the responsible doctor, who may optionally increase or decrease the dosage as necessary.

In some embodiments, the antibody compositions described herein are administered at a dose of 12 mg/kg weekly. In some embodiments, the antibody compositions described herein are administered at a 9 mg/kg loading dose followed one week later by 6 mg/kg weekly.

A therapeutically effective amount for cancer therapy may be measured by its ability to slow down or stabilize disease progression and/or ameliorate symptoms in a patient, and preferably to reverse disease progression, e.g., by reducing tumor size or preventing metastasis. The ability of an antibody or composition described herein to inhibit cancer may be evaluated by in vitro assays (e.g., as described in the Examples), as well as in suitable animal models that are predictive of the efficacy in human tumors (see, e.g., the Examples). Suitable dosage regimens will be selected in order to provide an optimum therapeutic response in each particular situation, for example, administered as a single bolus or as a continuous infusion, and with possible adjustment of the dosage as indicated by the exigencies of each case.

### Articles of Manufacture and Kits

The present invention also provides articles of manufacture and kits comprising an anti-EGFR composition as described herein. In some embodiments, the articles and kits are suitable for treating a patient as described herein, e.g., a mCRC patient from whom a tumor DNA sample lacks any combination of the genetic alterations described herein. For example, the articles and kits may be suitable for treating chemorefractory metastatic colorectal cancer in a patient with acquired resistance to other anti-EGFR antibody therapy, wherein a tumor DNA sample from the patient has a MAF of less than 20% for RAS, has a MAF of less than 0.1% for (e.g., is negative for) the BRAF mutation V600E, and has a MAF of less than 0.1% for (e.g., is negative for) the EGFR ECD mutations V441D, V441G, S464L, G465E, G465R, and S492R. In some embodiments, the pharmaceutically active ingredients in the articles and kits are prepared for administration at doses described herein, and are formulated for administration by methods described herein.

Unless otherwise defined herein, scientific and technical terms used in connection with the present invention shall have the meanings that are commonly understood by those of ordinary skill in the art. Exemplary methods and materials are described herein, although methods and materials similar or equivalent to those described herein can also be used in the practice or testing of the present invention. In case of conflict, the present specification, including definitions, will control.

Generally, nomenclature used in connection with, and techniques of, cell and tissue culture, molecular biology, immunology, microbiology, genetics, analytical chemistry, synthetic organic chemistry, medicinal and pharmaceutical chemistry, and protein and nucleic acid chemistry and hybridization described herein are those well-known and commonly used in the art. Enzymatic reactions and purification techniques are performed according to manufacturer's specifications, as commonly accomplished in the art or as described herein.

Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers.

All publications and other references mentioned herein are incorporated by reference in their entirety. Although a number of documents are cited herein, this citation does not constitute an admission that any of these documents forms part of the common general knowledge in the art.

In order that this invention may be better understood, the following examples are set forth. These examples are for purposes of illustration only and are not to be construed as limiting the scope of the invention in any manner.

### EXAMPLES

### Example 1: Protocols for Pre-Clinical and Clinical Studies of SYM004 Patient Selection

Male and female patients at least 18 years of age with histologically or cytologically confirmed mCRC that was exon 2 KRAS WT at the time of initial diagnosis, and who gave written informed consent, were screened for enrollment to the below-described trial. Patients were required to have demonstrated prior intolerance to or failure of standard first-line chemotherapy regimens including 5-FU, oxaliplatin, and irinotecan, and were allowed to have received bevacizumab or ziv-aflibercept. Prior therapy with regorafenib was not permitted. In addition, patients with acquired resistance to prior therapy with a marketed anti-EGFR mAb, as defined by having achieved either an objective partial response (PR), complete response (CR), or stable disease for >16 weeks followed by documented progressive disease (PD) during or within 6 months of completion of this therapy, were randomized within 6 months of their last therapy with either cetuximab or panitumumab. Patients were required to have measurable disease according to Response Evaluation Criteria in Solid Tumors (RECIST), a life expectancy of at least 3 months, and an ECOG performance status of 0 or 1. Further, patients were required to have acceptable organ function and to have recovered from toxicities associated with prior therapy, including a serum magnesium level of >0.9 mg/dL and skin rash of grade <1 according to the Common Terminology Criteria for Adverse Events (CTCAE) prior to enrollment. Patients who had hypersensitivity to any of the components of the investigational product or who had grade 3 or 4 hypersensitivity reactions during prior therapy with cetuximab or panitumumab were excluded.

### Study Design and Patient Populations Evaluated

This was a multinational, multicenter Phase 2 RCT (Sym004-05) in 254 patients with treatment-refractory mCRC and acquired resistance to therapy with anti-EGFR mAbs. In this study, patients randomly assigned to one of two dose regimens of Sym004, 12 mg/kg weekly (arm A, 83 patients) or 9 mg/kg followed by 6 mg/kg weekly (arm B, 86 patients) were compared to a control group, which included IC (investigator's choice) of capecitabine, 5-FU, or BSC (best supportive care) (arm C, 85 patients).

The study was designed to detect a 3 month improvement in OS (6 vs. 9 months) between arm A or arm B and arm C. A median OS of 6.8 months was reported for capecitabine in the same treatment line in an earlier study (Bendell et al., J. Clin. Oncol. 30:LBA3501-LBA3501 (2012)). Based on an assumed rate of accrual and a 5% drop-out rate the study analysis was scheduled for after 181 deaths had occurred. The primary analysis milestone for the trial was defined as the time at which at least 181 events (deaths) had been reported or 12 months after the last patient was randomized in the trial, whichever occurred later.

### Evaluation of Genetic Characteristics

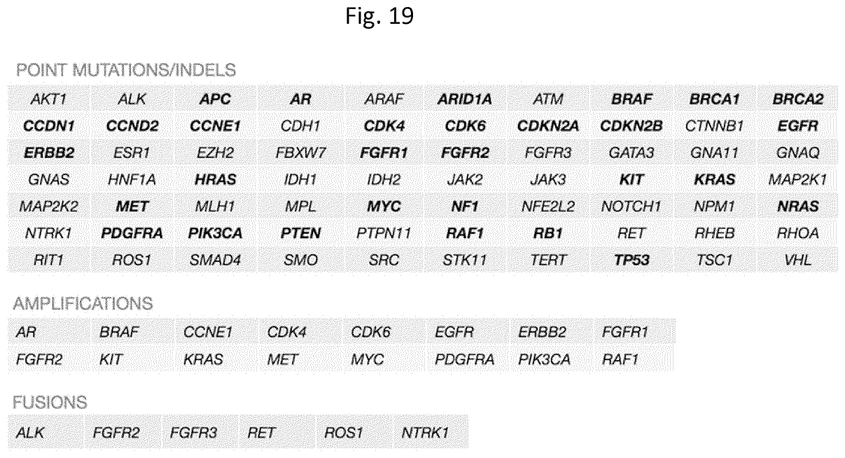

Baseline ctDNA profiles (Guardant360 version 2.9, Guardant Health) were obtained from blood samples collected from patients in the trial (N=193 patients). The Guardant360 assay version 2.9 (FIG. 19) sequences 70 genes, including all exons in 30 cancer genes, critical exons in 40 genes, amplifications (16 genes), and fusions (6 genes) in plasma samples derived from 10 mL of peripheral blood. The average depth of sequencing at each base is >10,000 times.

### Digital Droplet PCR Analysis of Serum Samples

Serial samples obtained prior to and after 3 weeks of treatment were analyzed for EGFR ECD mutation dynamics. Serum-isolated circulating free DNA was amplified using ddPCR™ Supermix for Probes with EGFR ECD mutation assays (Bio-Rad). ddPCR was then performed according to the manufacturer's protocol and the results reported as percentage or fractional abundance of mutant DNA alleles compared to total (mutant plus WT) DNA alleles. Droplets were generated using Auto-DG ddPCR system, where the reaction mix was added together with Droplet Generation Oil for Probes (Bio-Rad). Droplets were then thermally cycled under the following conditions: 5 minutes at 95° C., 40 cycles of 94° C. for 30 seconds and 55° C. for 1 minute, and finally 98° C. for 10 minutes (ramp rate 2° C./second). Droplets were analyzed with the QX200 Droplet Reader (Bio-Rad) for fluorescent measurement of FAM and HEX probes. Gating was performed based on positive and negative controls, and mutant populations were identified. The ddPCR data were analyzed with QuantaSoft analysis software (Bio-Rad) to obtain the fractional abundance of the mutated alleles in the WT or normal background. Quantification of the target molecule is presented as total number of copies (mutant plus WT) per sample in each reaction. The number of positive and negative droplets was used to calculate the concentration of the target and reference DNA sequences and their Poisson-based 95% CIs. Normal control DNA (from cell lines) and no DNA template controls were included in ddPCR analyses. Samples with too low positive events were repeated at least twice in independent experiments to validate the results obtained.

### Statistical Analyses

OS, defined as time from randomization to date of death or censored at last day of contact, was the primary efficacy endpoint. The Kaplan-Meier product-limit method was used to estimate median OS, and a Cox proportional hazard model was used to estimate the HR.

### In Vitro Antibody Binding Studies

A 4-fold serial dilution of unlabeled antibodies starting from 100 μg/mL was used to generate dose-response curves. Cells were transfected with WT EGFR or one of the different EGFR mutants 24 hours before 1 hour of incubation with the unlabeled antibodies. The cells were then washed and incubated with goat anti-human IgG (H+L)-Alexa Fluor® 647 (R&D Systems) at a 1:250 dilution for 30 minutes, before a fluorescence readout was measured using the iQue Screener platform (IntelliCyt). PBS+2% FBS was used as the incubation/washing buffer.

### Generation of Stable Cell Lines

NIH-3T3 cells (ATCC) stably overexpressing WT EGFR or EGFR ECD mutations were generated using retroviral transduction. Full length human EGFR constructs were synthesized (by Genscript) and subcloned into the retroviral vector pQCXIP (Clontech) using restriction enzymes NotI and AgeI. The presence of specific EGFR ECD mutations was confirmed by sequencing. A VSV-G pseudotyped retrovirus (Cell Biolabs) was produced using the Phoenix-AMPHO packaging cell line (ATCC), and filtered supernatants containing 8 μg/mL polybrene (Sigma) were used to infect NIH-3T3 cells. To generate stable pools, the cells were selected in 2 μg/mL puromycin (Thermo Fisher) for 7 days.

### Cell Viability Assay

The cell lines DiFi and DCR7 (S492R) were obtained as previously described (Sanchez-Martin et al., supra). 2-fold serial dilution of antibodies starting from 25 μg/mL was used to generate dose-response curves for EGFR ECD mutated DiFi and DCR7 cell lines. Cells were cultured in the presence of antibodies in medium containing 2% FBS. After 96 hours of culture, cell viability was determined using the WST-1 assay (Roche Diagnostics).

### Transient Transfection

A standard Lipofectamine 2000 transfection protocol was used to transiently transfect NIH-3T3 cells with plasmids containing either human full length EGFR or the human EGFR ECD. A control plasmid containing green fluorescent protein was used to determine the transfection efficiency. After 48 hours, the cells were used for experiments.

Quantification of Total EGFR and pEGFR Levels by Simple Western

Lysates were generated using Pierce RIPA buffer containing protease inhibitors (Thermo Scientific) and phosphatase inhibitors (Calbiochem). Samples for Simple Western analysis were diluted to 0.2 μg/μL in a master mix containing internal fluorescent standards and reducing agent, and were processed per standard protocol using a Sally Sue instrument (ProteinSimple). Antibodies against total EGFR (C74139), pEGFR (Y1068), and Pan-Actin (all from Cell Signaling Technology) were diluted 1:50.

In vivo Experiments

Five-week-old male BALB/c nude mice were purchased from Charles River Laboratories and housed under standard conditions in the pathogen-free animal facility at the Barcelona Biomedical Research Park (PRBB). Mice were treated humanely and with regard for alleviation of suffering according to institution-approved protocols. 5×10⁶ cells suspended in sterile PBS containing 50% Matrigel (BD Biosciences) were injected subcutaneously into the mouse flanks. Tumor volume was determined biweekly from caliper measurements of tumor length (L) and width (W) according to the formula L×W²/2. Tumors were allowed to grow until the volume reached approximately 200-300 mm³. Mice were randomized to 6 groups with 5 mice in each group. 3 groups were injected with DiFi and 3 groups with DCR7. Treatment groups consisted of DiFi control and DCR7 control (both IgG isotype control), DiFi+cetuximab (40 mg/kg), DCR7+cetuximab (40 mg/kg), DiFi+Sym004 (40 mg/kg), and DCR7+Sym004 (40 mg/kg). Mice were treated intraperitoneally twice a week.

Analysis of the Patient Subgroup Excluded Due to Medical Practice Inconsistent with the Standard Therapy of Patients with mCRC

For patients enrolled in the Sym004 Phase 2 study, there was a notable disparity in OS between patients treated in Russia and those treated in other countries. Median OS for all treatments combined was 8.9 months for all patients excluding those in Russia (i.e., the EU and US only) vs. 13.9 months in Russia. The median duration of treatment (all arms) was nearly 4 times longer for patients from Russia (36 months) than for the EU and US patients (9.1 months). Also, 25% of the EU and US patients had EGFR ECD mutations vs. none of the patients from Russia. Because of these disparities, ad hoc analyses excluding patients enrolled by the Russian sites were done to remove this confounding country effect. The data obtained support the suggestion that the patients in Russia were more sensitive/less refractory to therapy in general and to treatment on the three arms of this protocol specifically.

### Antibody-Dependent Cellular Cytotoxicity (ADCC)

The indicated cell lines were loaded with ⁵¹Cr for 1 hour and were then incubated with single antibodies or antibody mixture (50 μg/ml) and isolated natural killer (NK) cells for 4 hours. Levels of ⁵¹Cr in the supernatant were measured using MicroBeta2 (PerkinElmer). Specific lysis was calculated as a percentage of maximum lysis with spontaneous lysis subtracted. The use of human donors followed ethical permission.

### PDX Models

CRC PDX tumor xenografts were derived from surgical specimens from cancer patients and were established and characterized at EPO-GmbH, Germany or Oncotest, Germany. After transplantation of 2×2 mm tumor fragments to NMRI-Foxn1nu mice, tumors were measured at least twice weekly. When tumors reached 50-250 mm³, preferably 80-200 mm³, animals were distributed into experimental groups with the aim of having comparable median and mean group tumor volumes of approximately 100-200 mm³, and treatment was initiated. The experiment was performed with 10 animals/group and three groups/model: vehicle control, Sym004, and cetuximab. Sym004 and cetuximab were administered at a dose of 30 mg/kg intraperitoneally (i.p.) twice weekly for 5 weeks (9-10 doses in total).

### Example 2: Patients in Clinical Study of Sym004

A total of 299 patients were screened. 254 of them were eligible and were defined as the intent-to-treat (ITT) population. Patients were randomly allocated to three treatment arms: a Sym004 regimen of 12 mg/kg weekly (12, arm A), Sym004 as a 9 mg/kg loading dose followed by 6 mg/kg weekly (9/6, arm B), or IC of capecitabine, 5-fluorouracil (5-FU), or BSC (arm C). Of the eligible patients, 245 (96.5%) received the study-designated therapy, with 2 patients in arm B and 7 patients in arm C withdrawing prior to receiving treatment. Of the 254 randomized patients, 30 patients were excluded from efficacy analyses due to major study protocol violations of patient inclusion/exclusion criteria (See above: “Analysis of the Patient Subgroup Excluded due to Medical Practice Inconsistent with the Standard Therapy of Patients with mCRC”). Patients were well-balanced among the three treatment groups, with no evidence of disparities in mean age, sex distribution, race, or Eastern Cooperative Oncology Group (ECOG) performance status. In particular, the time since the last prior therapy, the number of prior therapies, and prior therapy with either cetuximab or panitumumab anti-EGFR mAbs did not show any imbalances.

The clinical study shows that Sym004's adverse event (AE) profile was consistent with other anti-EGFR mAbs, although the frequency and severity of both dermatologic AEs and hypomagnesemia were higher. This trial only included patients who had benefited from prior therapy with cetuximab or panitumumab and they hence also had a higher incidence of skin toxicity. However, the frequency of gastrointestinal (GI) AEs appeared to be lower than has been reported for cetuximab or panitumumab.

### Example 3: Baseline Biomarker ctDNA Analysis

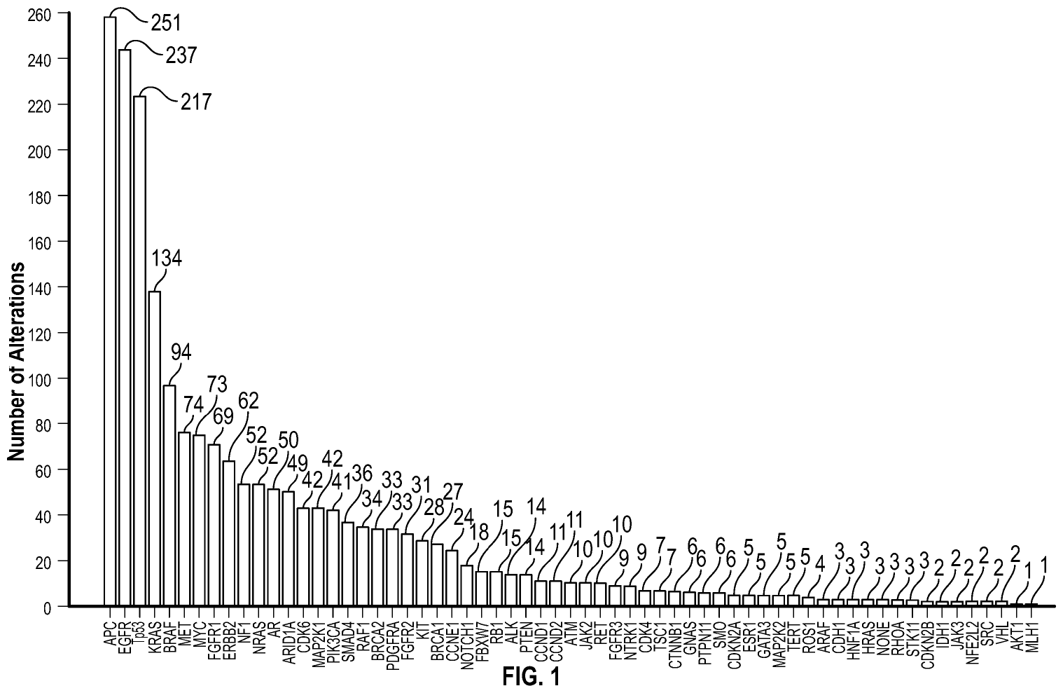

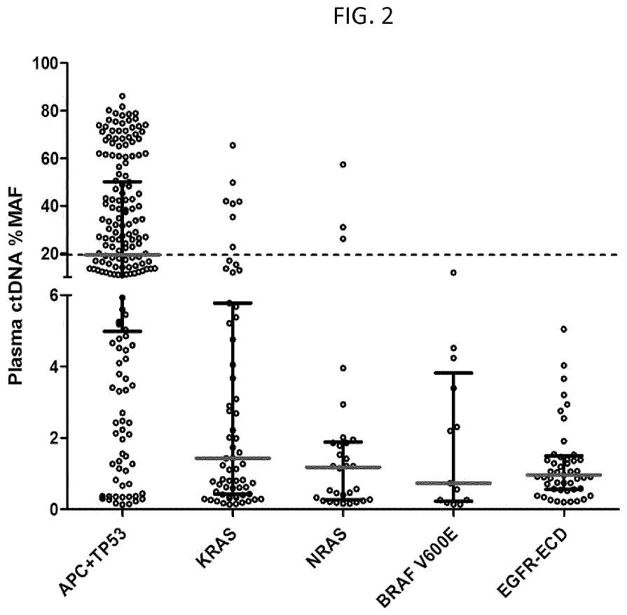

Over the last decade, studies have elucidated the factors responsible for de novo and acquired resistance to anti-EGFR mAbs and have clearly documented both the heterogeneity of tumors as well as their complex genetic evolution in the presence of EGFR pathway inhibition. Genotyping of baseline ctDNA confirmed previously reported mechanisms of acquired resistance to cetuximab and panitumumab, including BRAF V600E (6.7%), RAS (29.5%) and the EGFR ECD (25%) mutations, as well as amplification of ERBB2 and MET (FIGS. 1 and 2).

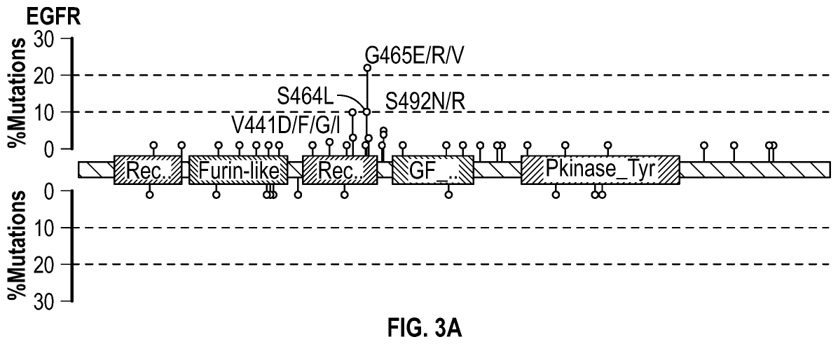

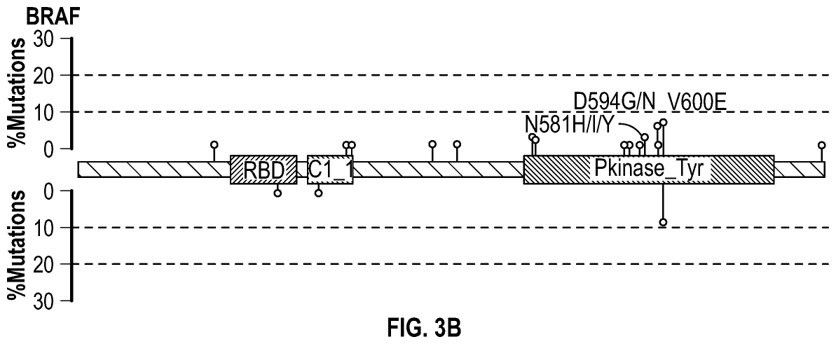

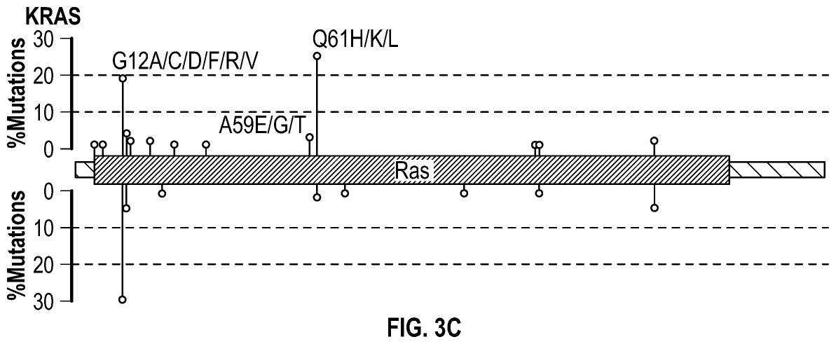

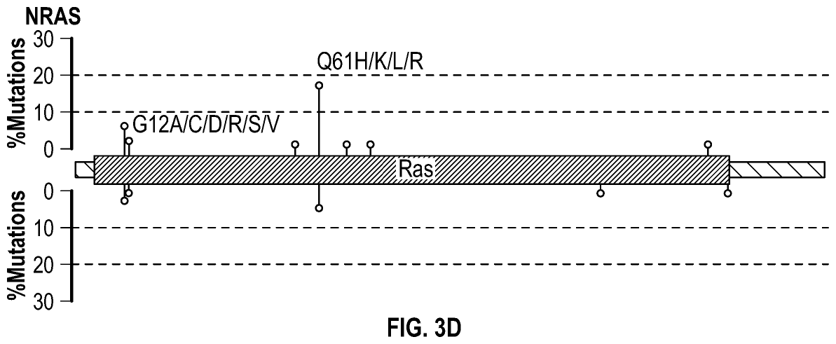

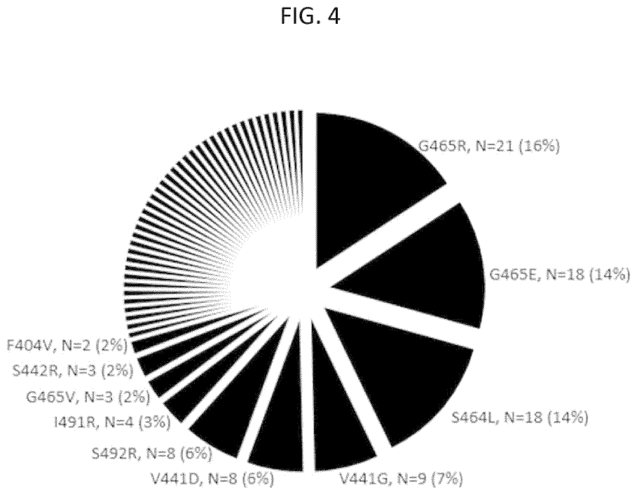

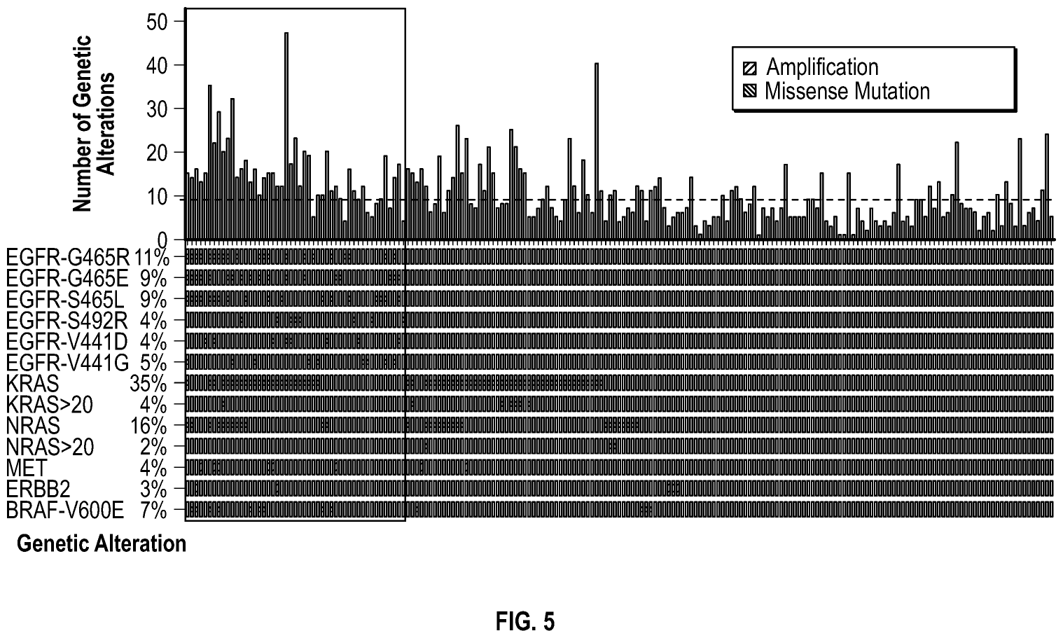

Inactivation of APC and/or TP53 is an early event in the development of CRC and the highest mutant allele frequency (MAF) APC/TP53 alteration in a patient's ctDNA can therefore serve as an arbitrary marker for clonal mutations (present in all tumor cells). FIG. 2 depicts MAF for the most prevalent TP53, APC, KRAS, and NRAS mutations, as well as BRAF V600E, in the 193 patients. The median MAF for the most prevalent TP53/APC alterations was close to 20%, suggesting that mutations with a MAF above 20% are clonal. The median MAFs for KRAS, NRAS, and BRAF were much lower than 20%, indicating that these mutations are primarily subclonal, although a subset of 10 patients harbored RAS mutations at allele frequencies above 20%. Of note, analysis of specific missense mutations in ctDNA confirmed the differential genomic landscape of primary vs. acquired resistance to anti-EGFR therapy, including the predominance of RAS Q61 and EGFR ECD mutations compared to data from the Cancer Genome Atlas (TCGA) from patients naïve to anti-EGFR therapies (FIGS. 3A-3D). Six EGFR ECD mutations (V441D, V441G, S464L, G465E, G465R and S492R) impacting four distinct amino acid positions of the EGFR ECD were frequent among the anti-EGFR-refractory patients (FIG. 4). Comprehensive liquid profiling of genetic biomarkers in the 193 mCRC patients revealed cancer cell clonal evolution and high intrapatient heterogeneity following anti-EGFR therapy, reflecting genomic complexity (FIG. 5).

### Example 4: Impact of EGFR ECD Mutations on Anti-EGFR Antibody Activity

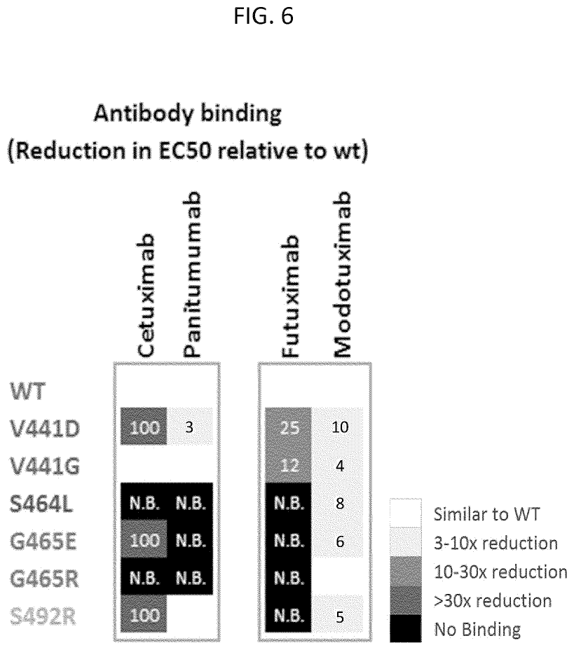

The four most frequently mutated EGFR positions (which harbor the six mutations V441D, V441G, S464L, G465E, G465R and S492R) are located at the surface of domain III of EGFR and are tightly clustered in an area known to be involved in cetuximab and panitumumab binding (Voigt et al., Neoplasia 14:1023-1031 (2012) and Sickmier et al., PLoS One 11:e0163366 (2016)). Impaired binding of cetuximab and panitumumab to specific EGFR mutants was confirmed (FIG. 6). Binding of cetuximab to all EGFR ECD mutants, with the exception of the V441G mutant, was reduced compared to WT EGFR. Similar results were obtained for panitumumab, with a reduction in binding to all mutants apart from S492R and V441G. Futuximab, which binds to an epitope in domain IIIB of EGFR, also had significantly reduced binding to the majority of the EGFR ECD mutants. In contrast, binding of modotuximab, which binds to a different region on domain III of EGFR, was largely unaffected by the EGFR ECD mutations (FIG. 6).

To investigate how the observed changes in antibody binding affect the functional activity of the antibodies, cells overexpressing WT EGFR or representative EGFR ECD mutations were generated.

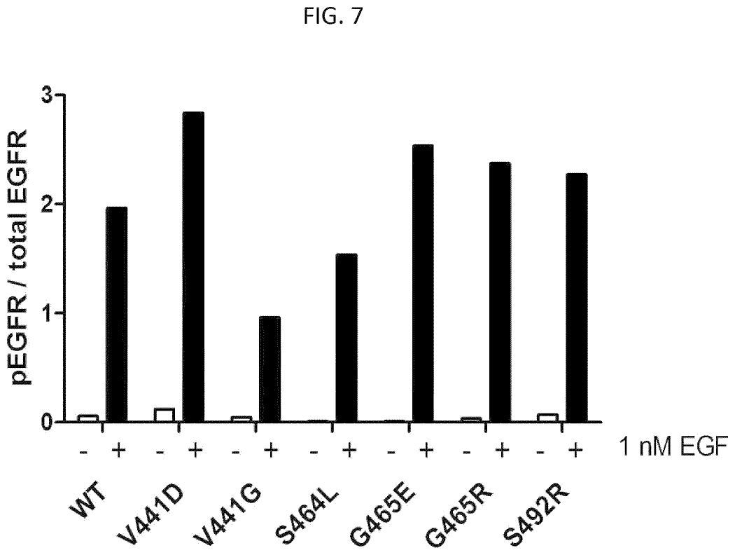

All EGFR ECD mutant cell lines showed similar basal EGFR phosphorylation levels as the EGFR WT expressing cell line and retained the ability to be activated by EGF (FIG. 7), suggesting that the EGFR ECD mutations per se do not have a major impact on the activity of the receptor.

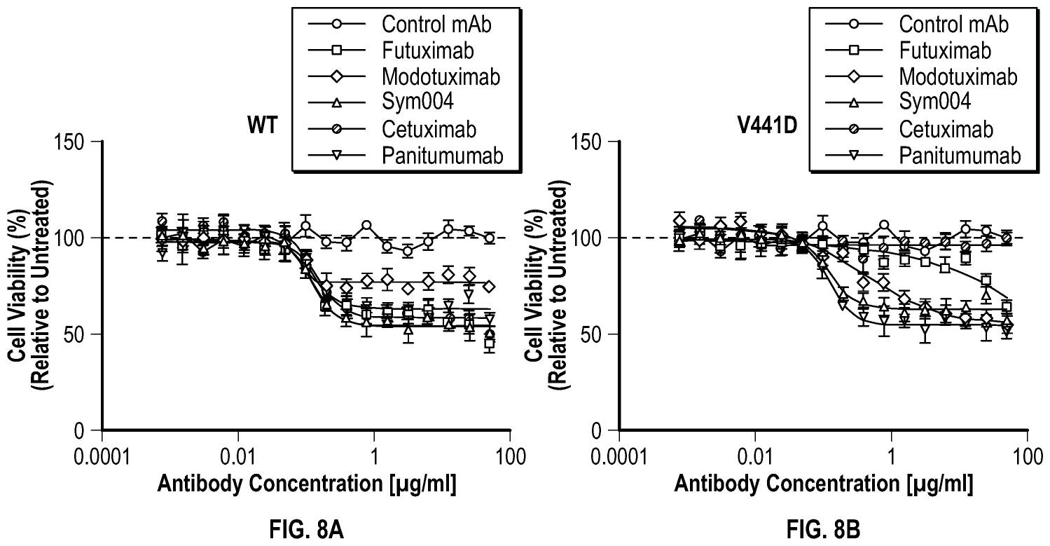

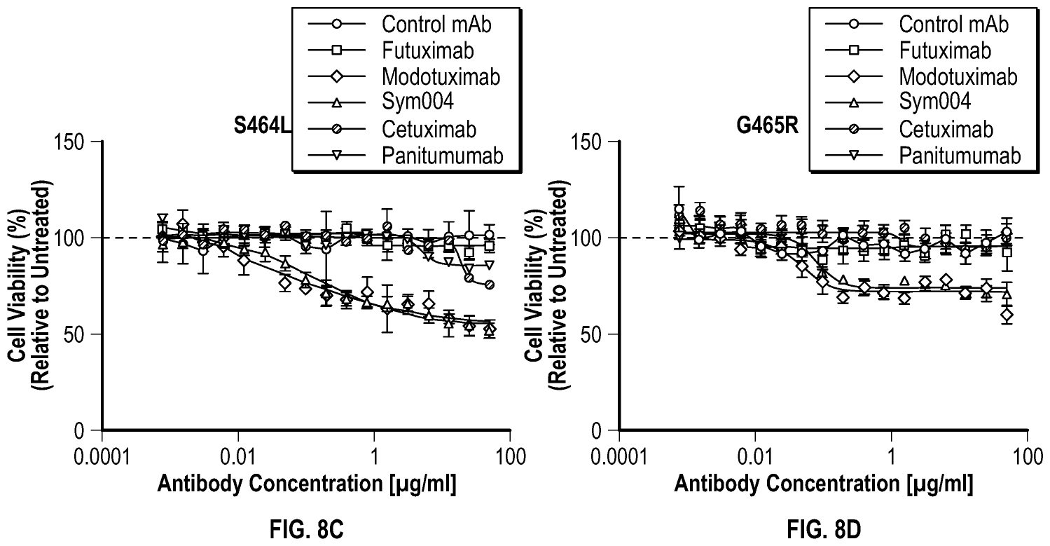

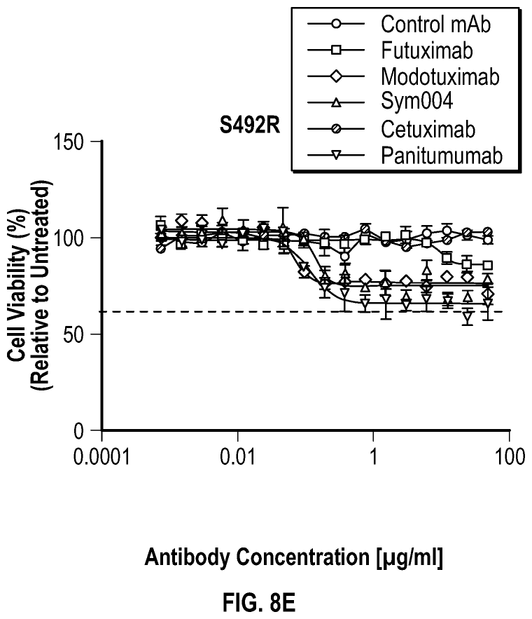

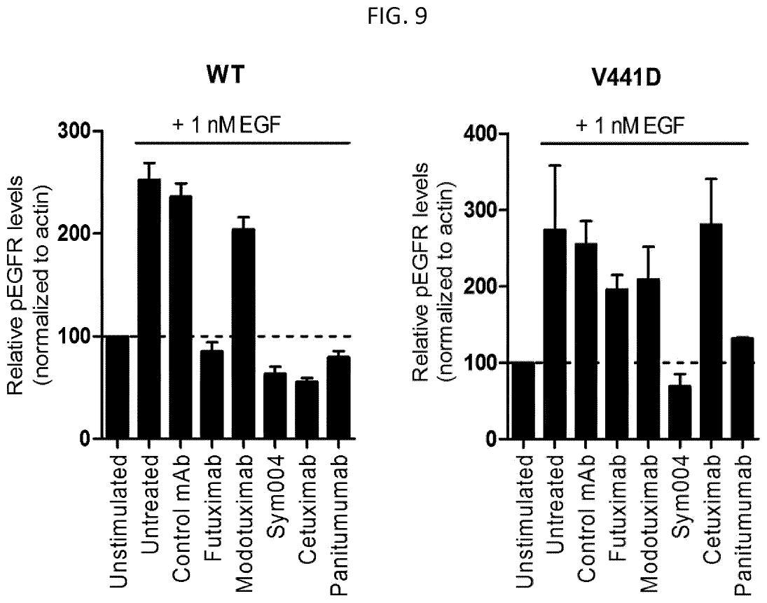

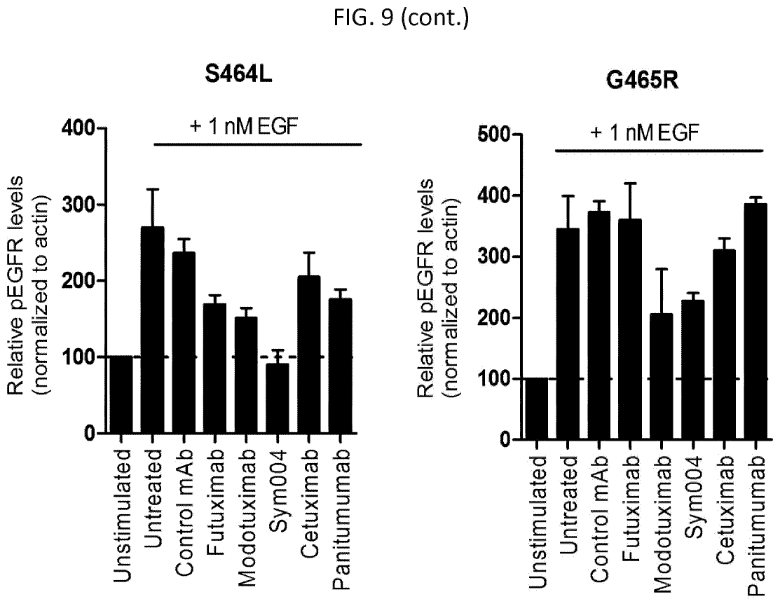

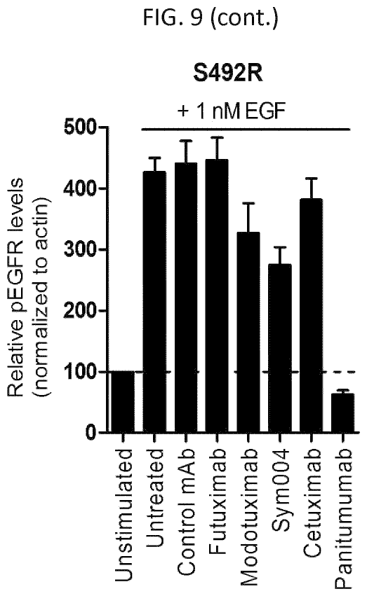

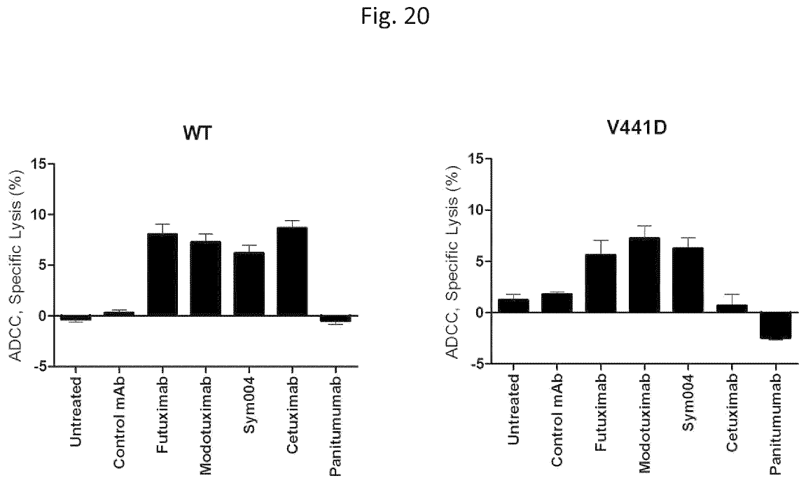

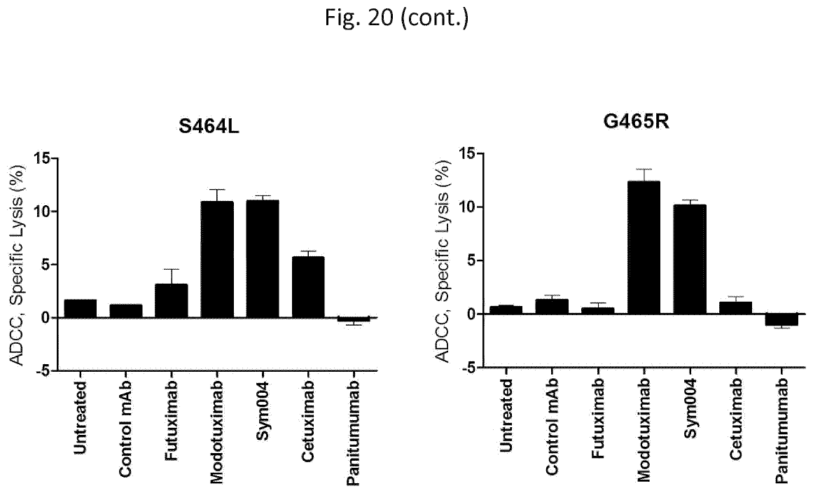

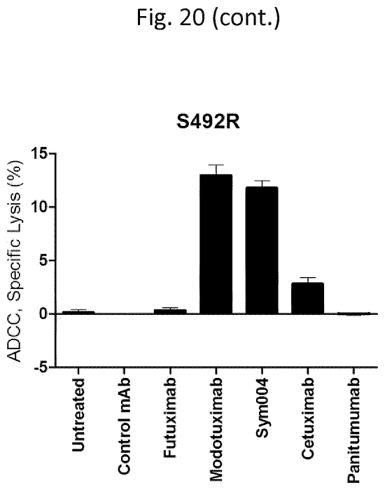

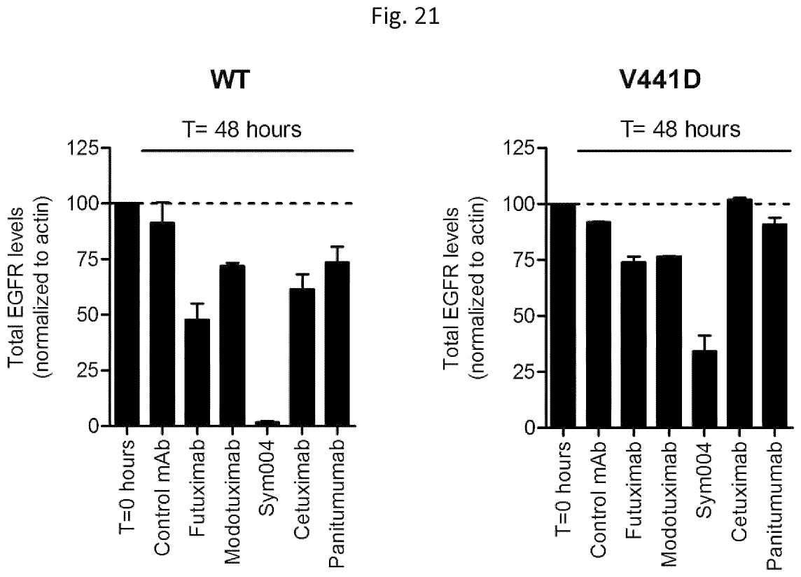

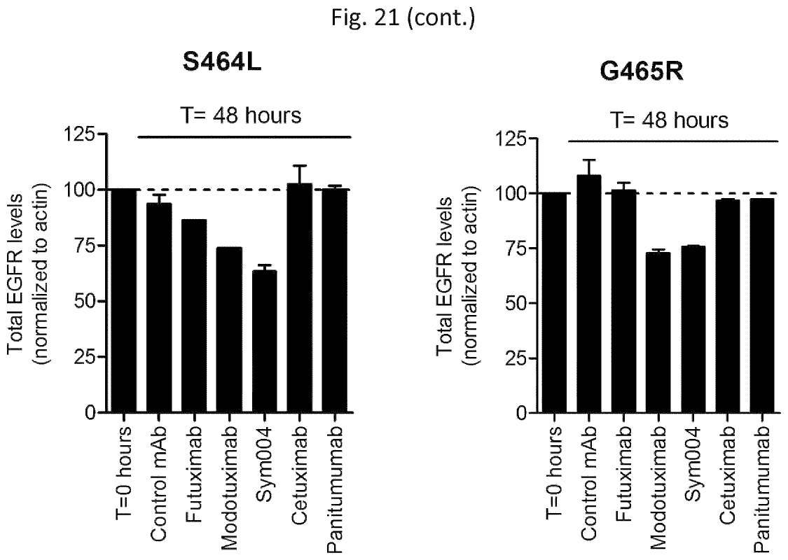

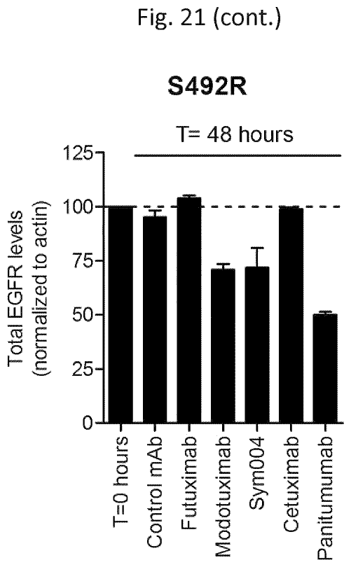

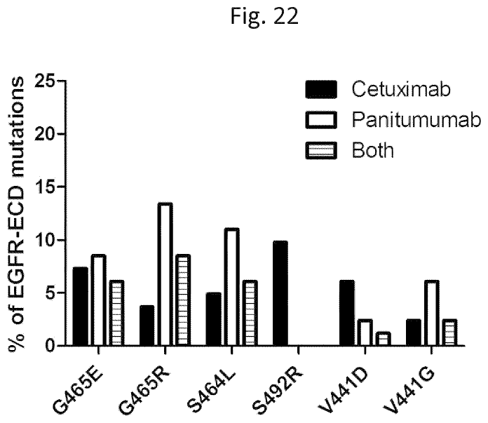

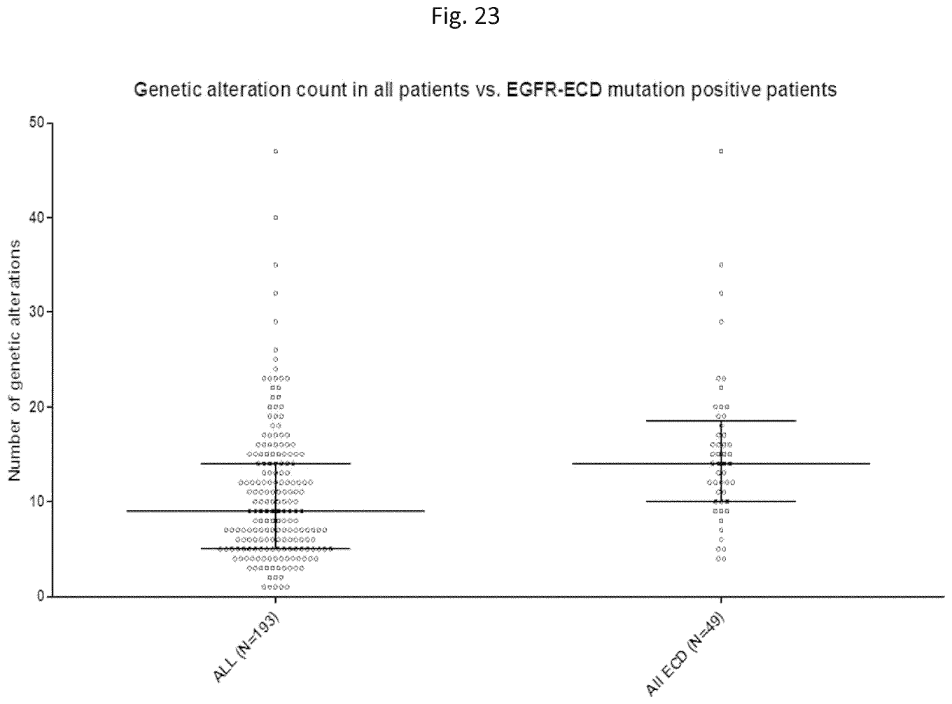

Next, the effect of Sym004, the individual Sym004 mAbs, cetuximab, and panitumumab on cell viability, EGF induced receptor phosphorylation, and antibody-dependent cellular cytotoxicity (ADCC) was determined. In line with the binding data, cetuximab had no or very little impact on the viability of cells expressing any of the four mutant receptors, failed to block EGF induced receptor phosphorylation, and failed to induce ADCC (FIGS. 8, 9, and 20). The functional activity of panitumumab was impaired by the S464L and G465R mutations. As expected, panitumumab (IgG2) did not induce ADCC (FIG. 20). All the EGFR ECD mutations were detrimental to the functional activity of the futuximab component of Sym004 but did not impact the ability of the modotuximab component to reduce cell viability, block EGF induced phosphorylation, or induce ADCC (FIGS. 8, 9, and 20). As expected, the lack of binding and function of the futuximab component resulted in a Sym004 activity profile that was identical to that of modotuximab. EGFR down-modulation, a hallmark of synergistic Sym004 activity, was determined in all the mutant cell lines. The data show that Sym004 induced extensive degradation of WT EGFR but failed to induce significant degradation of the mutated receptors (FIG. 21).

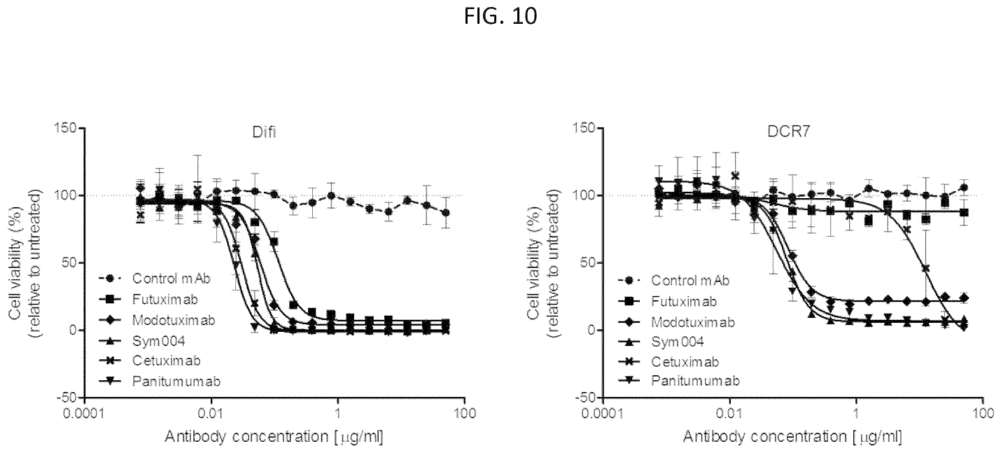

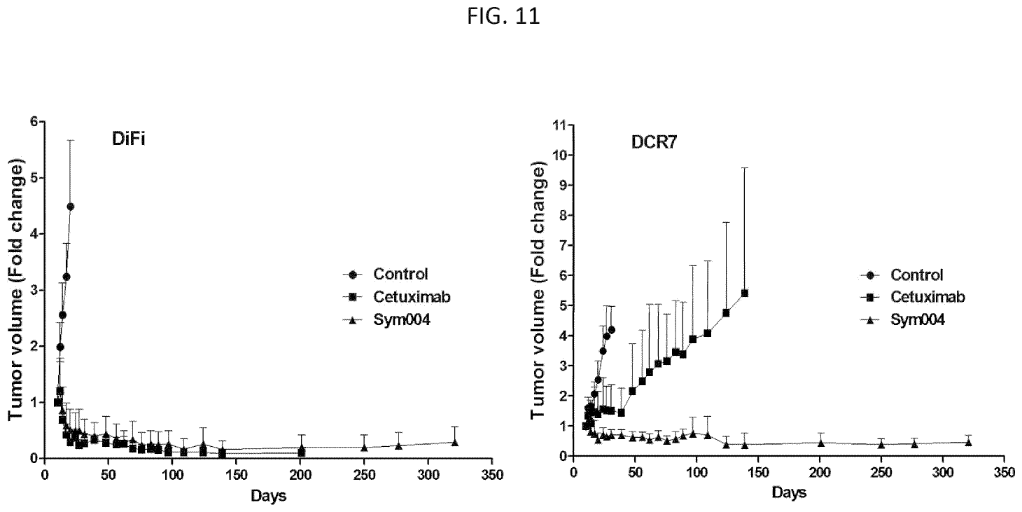

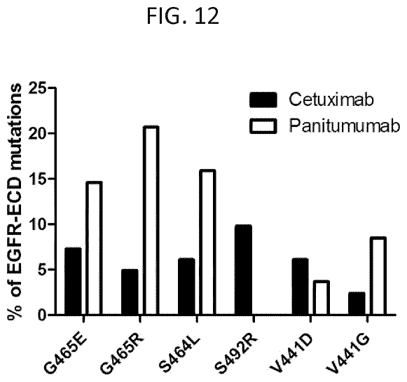

The S492R mutation has also emerged upon continuous exposure of the cell line DiFi to cetuximab (DCR7 cells), offering an interesting preclinical model to assess the cell viability effects of Sym004 in vitro and in vivo. Sym004 and its individual component antibodies showed similar effects on cell viability of EGFR WT parental DiFi cells in vitro (FIG. 10). In contrast, only the mAbs with evidence of strong binding to the EGFR ECD mutant S492R (modotuximab and panitumumab, as well as Sym004 due to the modotuximab component) caused profound and sustained decreases in the viability of DCR7-S492R mutant cells. Despite the lack of binding and activity of the futuximab component, Sym004 eradicated DCR7-S492R mutant tumors, demonstrating that the modotuximab component has ample activity in this model due to its direct effect on the mutated receptors and ADCC induction (FIG. 11).

Taken together, these data show that Sym004 remains partially inhibitory in vitro and in vivo of EGFR with ECD mutations at surface exposed amino acids in the V441-S492 region of domain III. This is due to rescue by the modotuximab component, which retains full binding and activity towards the most frequent EGFR ECD mutations in mCRCs.

### Example 5: EGFR ECD Mutations in Patients Treated with Sym004

The six most frequent EGFR ECD mutations (V441D, V441G, S464L, G465E, G465R and S492R) were detected in baseline ctDNA from 25% of the 193 patients included in the genetic characteristic study. EGFR ECD mutations occurred more frequently in patients who received panitumumab as last treatment (40% of 68 patients) compared to patients who received cetuximab as last treatment (18% of 125 patients). Time since last anti-EGFR treatment (cetuximab or panitumumab) was similar for the EGFR ECD mutated patients and the genetic characteristic profiled patients as a whole. Of note, 35 patients had previously received both cetuximab and panitumumab at different time points during the course of their disease, and the rate of EGFR ECD mutations in ctDNA in this subset of patients was 39%. The most frequent EGFR ECD mutations following panitumumab therapy were G465R/E and S464L, whereas in patients treated with cetuximab the most frequently detected mutation was S492R. The S492R mutation only emerged in patients treated with cetuximab (Montagut et al., supra; Arena et al., supra; and Newhall et al., Ann. Oncol. 25:ii109 (2014)).

### Example 6: Molecular Subsets Predictive of Sym004 Clinical Efficacy

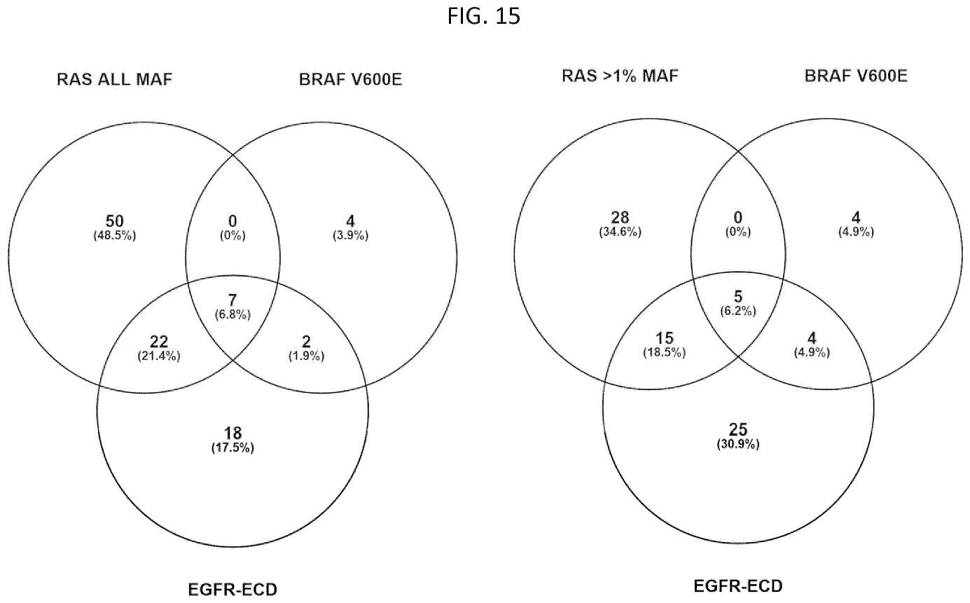

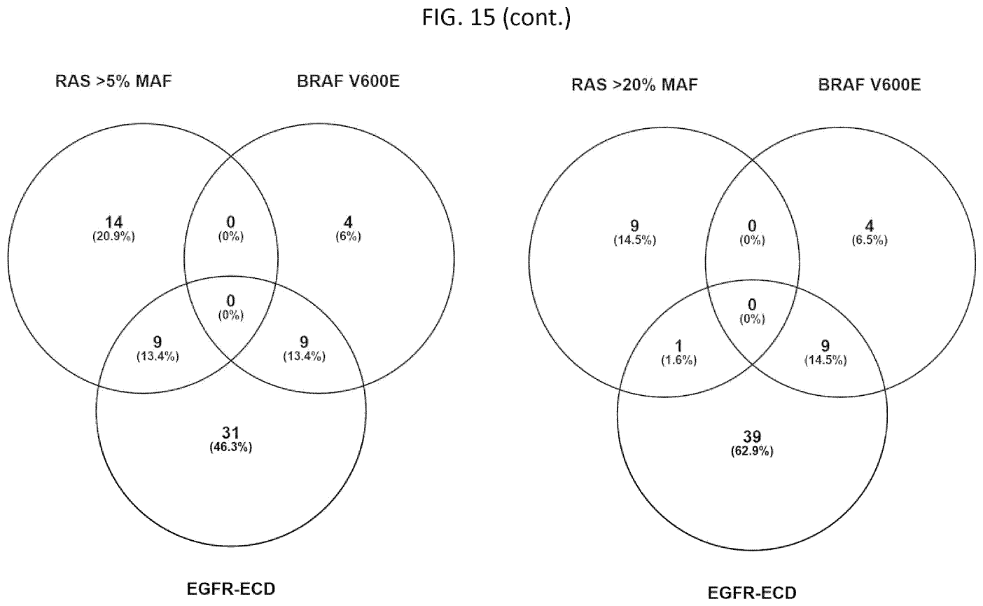

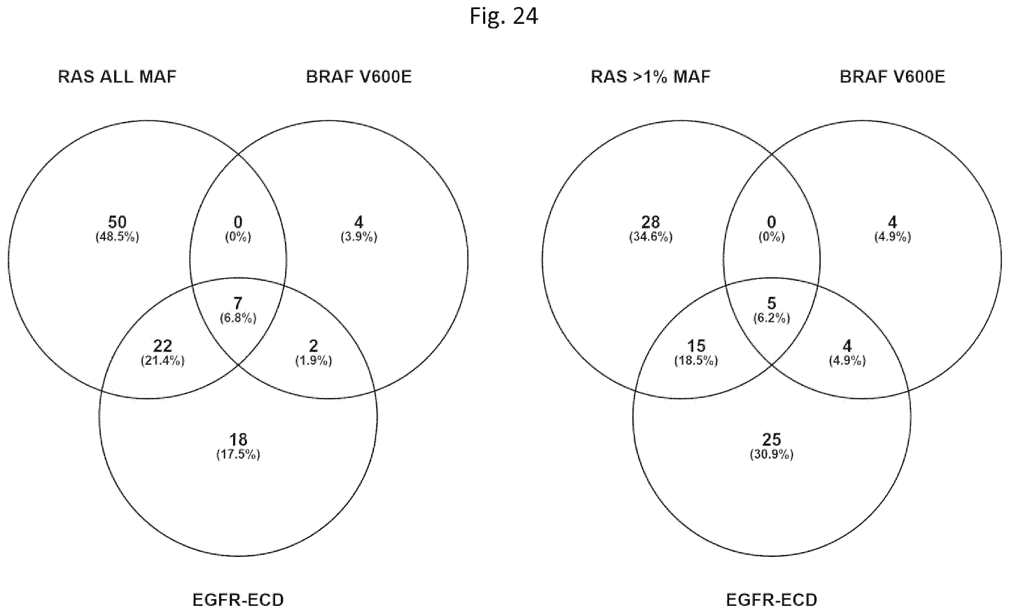

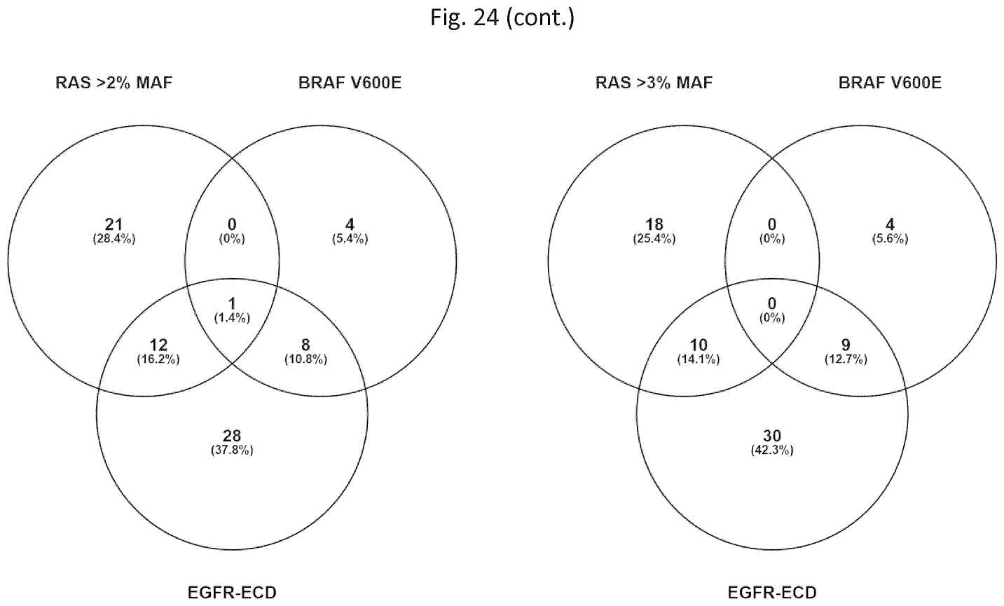

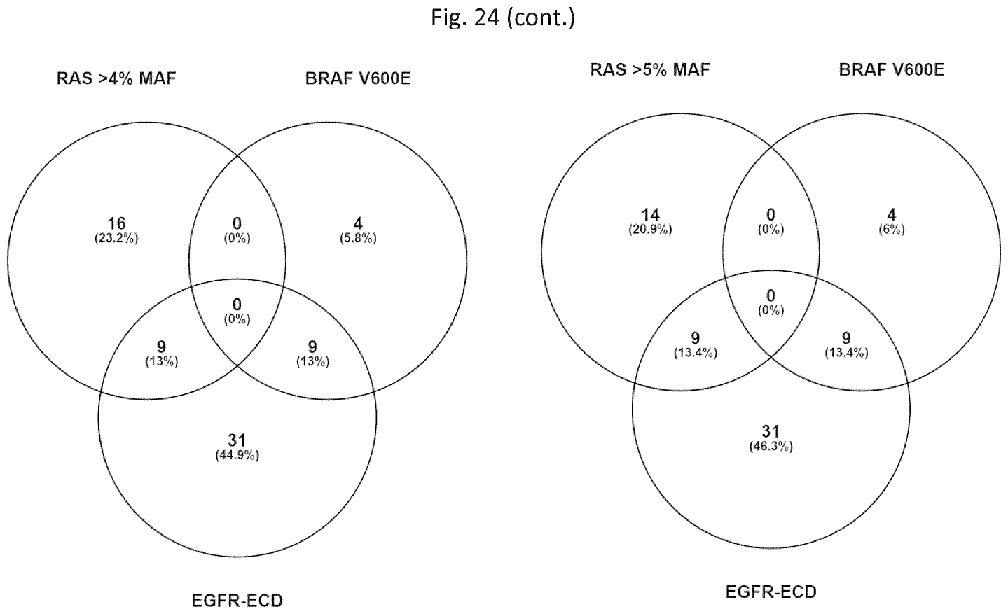

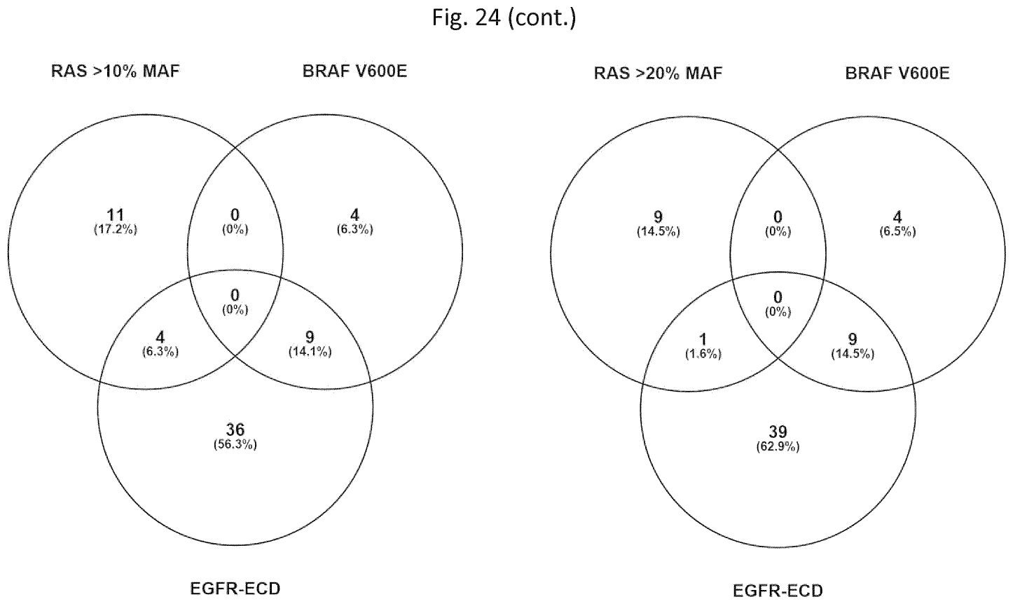

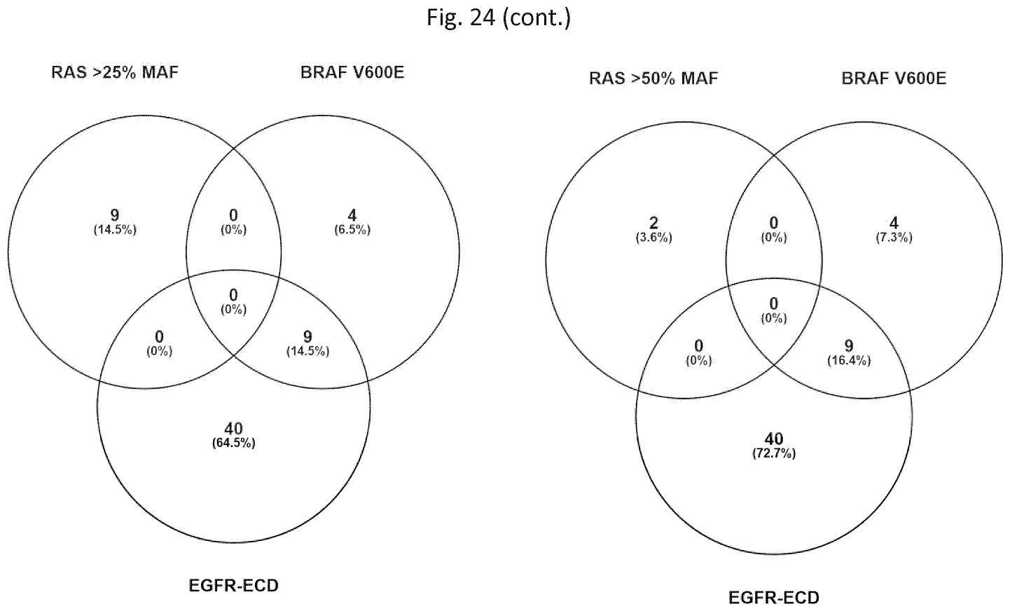

We aimed to define molecular subgroups that would predict Sym004 efficacy, as a predefined exploratory secondary objective of the study. In particular, we analyzed mutations in three genes often mutated in cancer patients, RAS, BRAF, and EGFR. First, in order to help define a clinically relevant mutational cut-off, we analyzed the overlap between different MAFs at baseline (FIGS. 15 and 24). A very high MAF (>20%) was detected in 10 patients (3 for NRAS mutations and 7 for KRAS mutations; 5% of the overall study population). With one exception, these high percentage RAS mutations did not coexist with other resistance mutations (FIG. 15). They potentially existed at diagnosis and defined a subgroup of patients. Of note, none of the EGFR ECD mutations had a MAF of >20%.

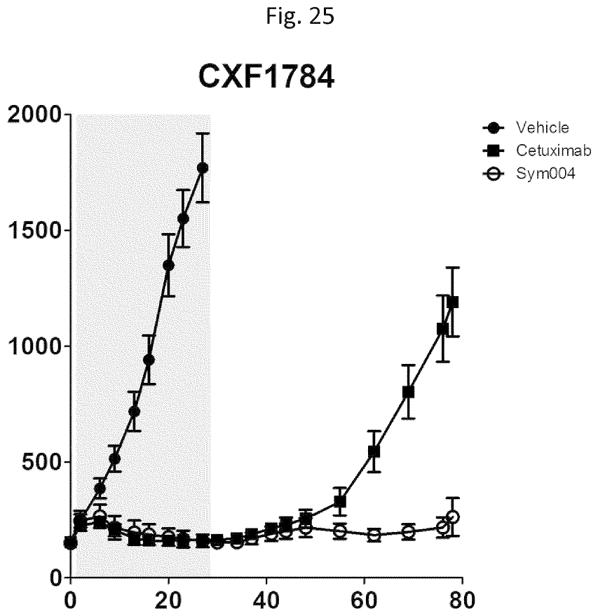

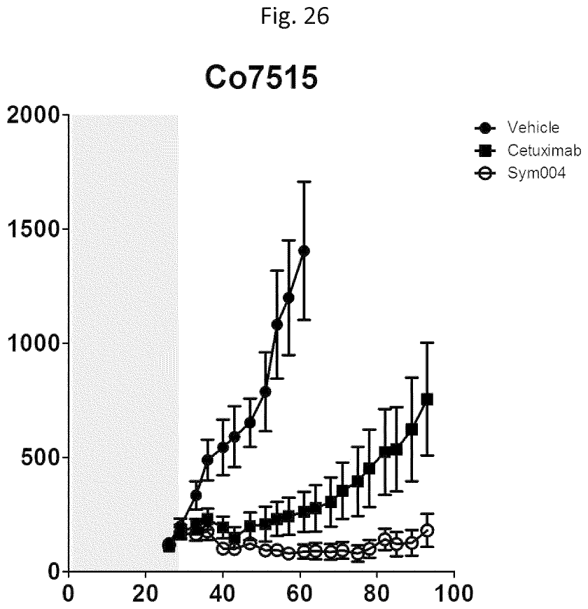

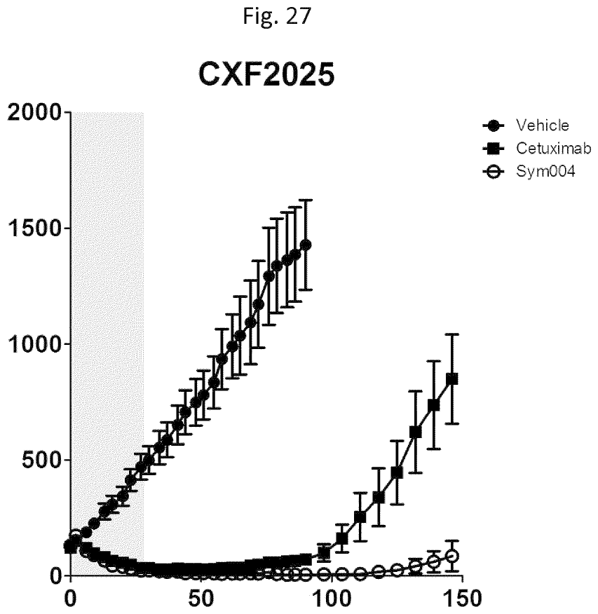

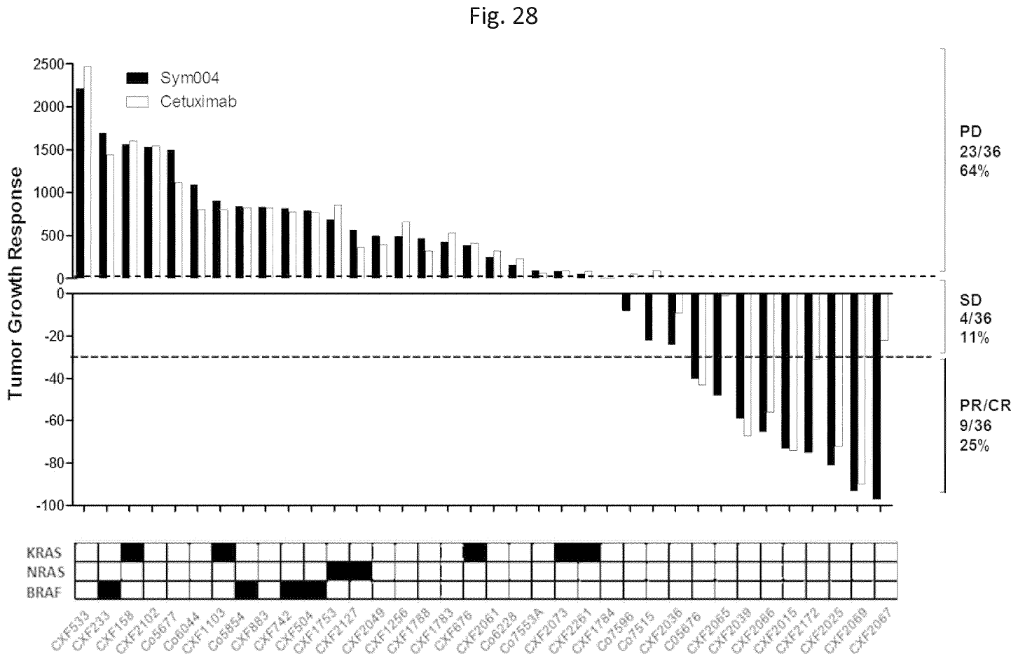

In pre-clinical patient-derived xenograft (PDX) CRC models with KRAS, NRAS, and BRAF V600E mutations, no or limited Sym004 activity was observed, indicating that mutations in these genes cause resistance to Sym004 (FIGS. 25-28).

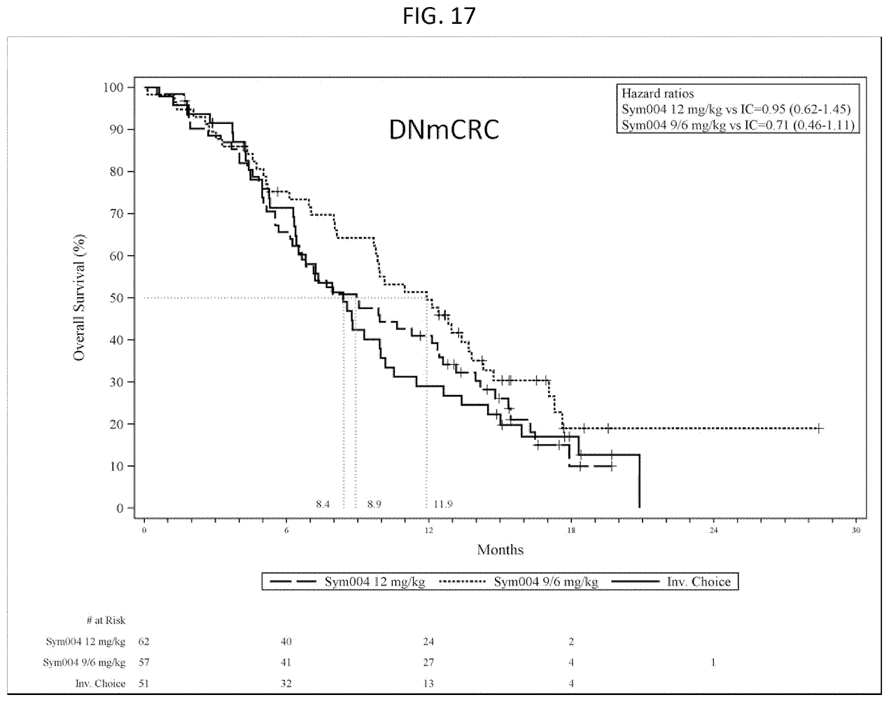

Efficacy analysis in a genomically-defined subpopulation, which included patients who had a RAS MAF of <20% (5% of patients in the study) and were negative for the BRAF V600E mutation (6.7% of patients in the study) (named double-negative mCRC, DNmCRC; 170 patients), showed a one-year survival rate of 50% (95% CI 36-62) for the Sym004 9/6 group (arm B), 41% (95% CI 29-53) for the Sym004 12 group (arm A), and 29% (95% CI 17-43) for the IC group (arm C). Further, the analysis showed an increase in OS of 2 months (11.9 vs. 9.9 months) for the Sym004 9/6 group (arm B) and a smaller increase in the Sym004 12 group (arm A) (8.9 vs. 7.7 months) compared to the ITT population as a whole. OS in the IC population (arm C) was unchanged (8.4 vs. 8.5 months in the ITT population and DNmCRC subgroup, respectively). Thus, this subset analysis showed an increase in median OS of as many as 3.5 months in the DNmCRC population of patients treated with Sym004 9/6 (11.9 months) compared to patients randomized to IC (8.4 months) (FIG. 17).

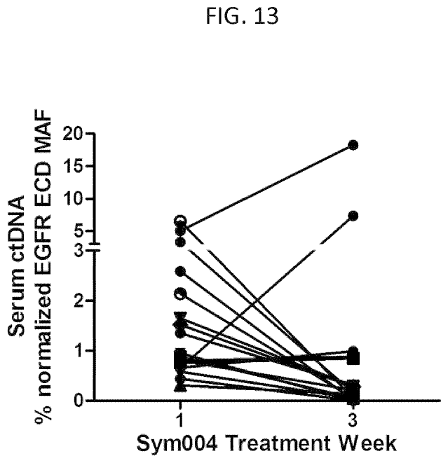

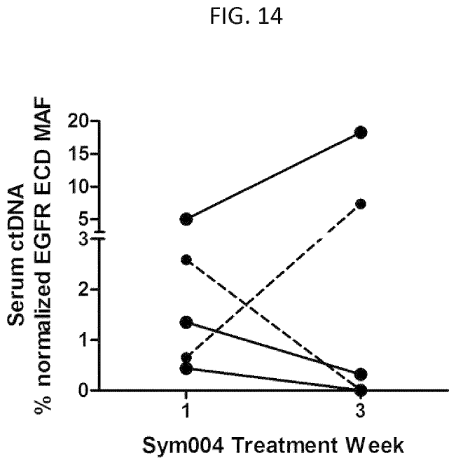

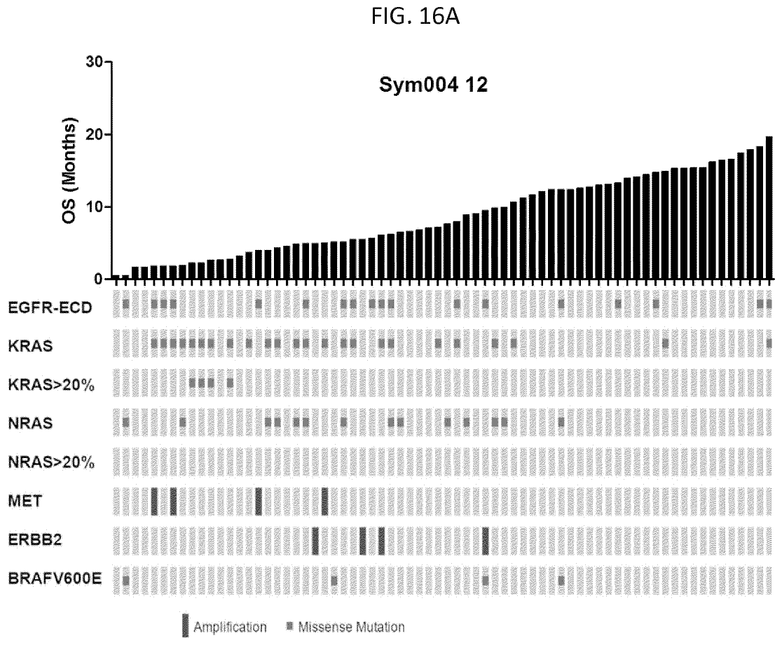

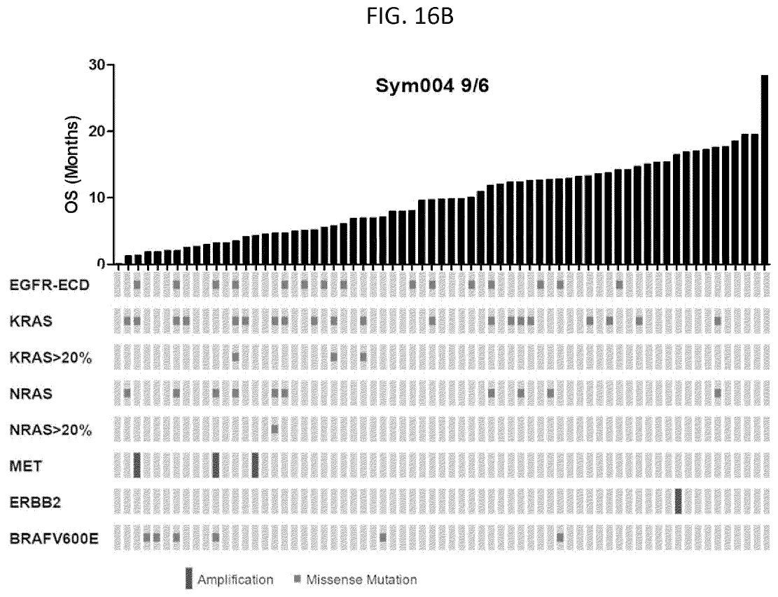

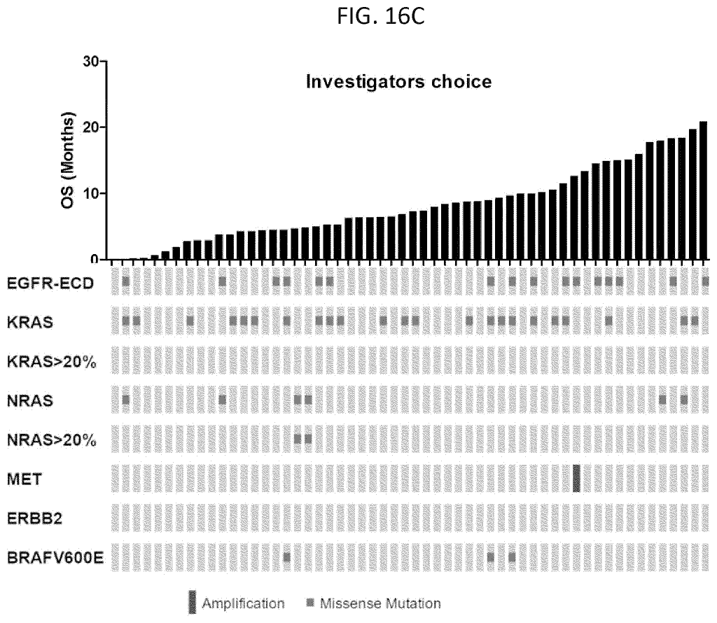

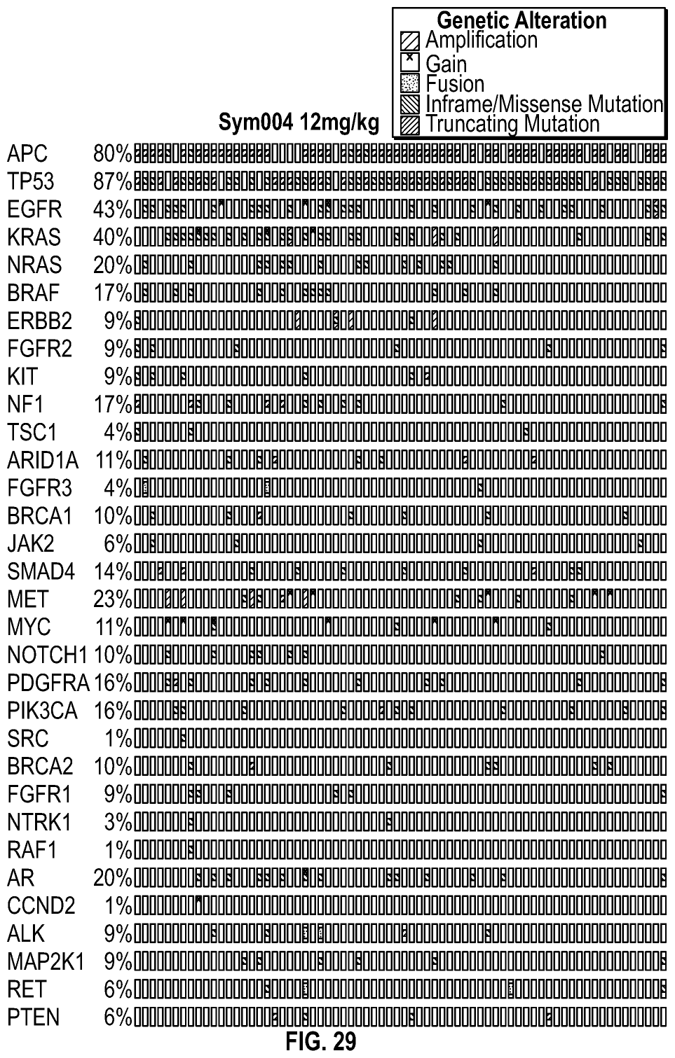

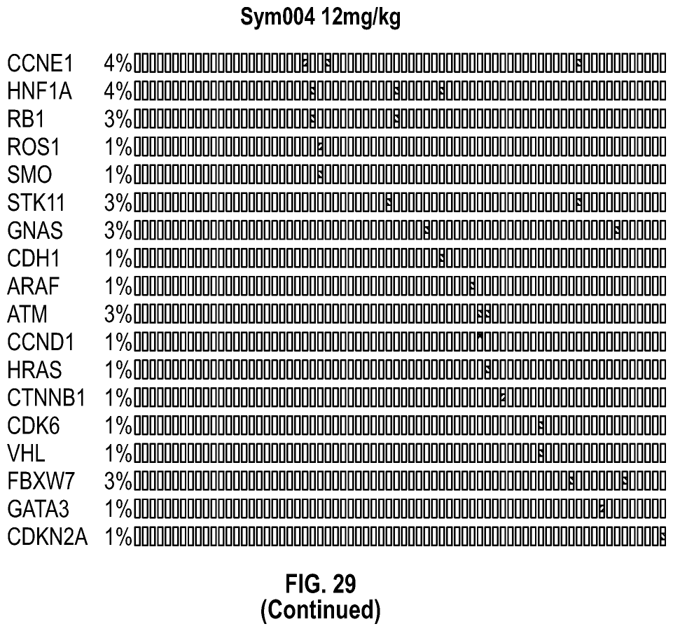

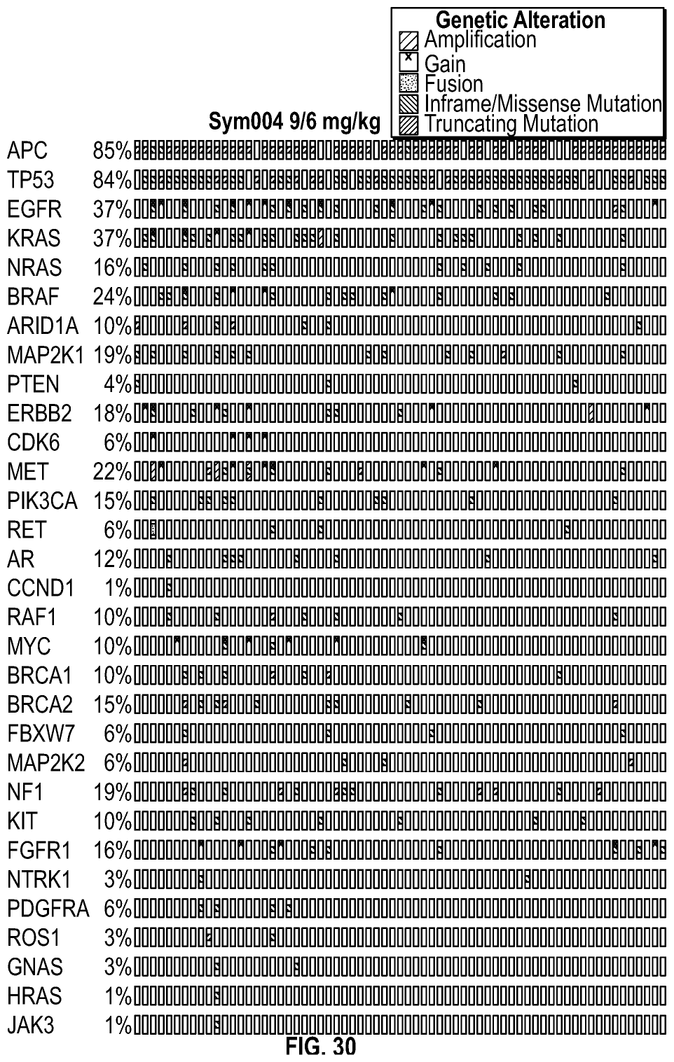

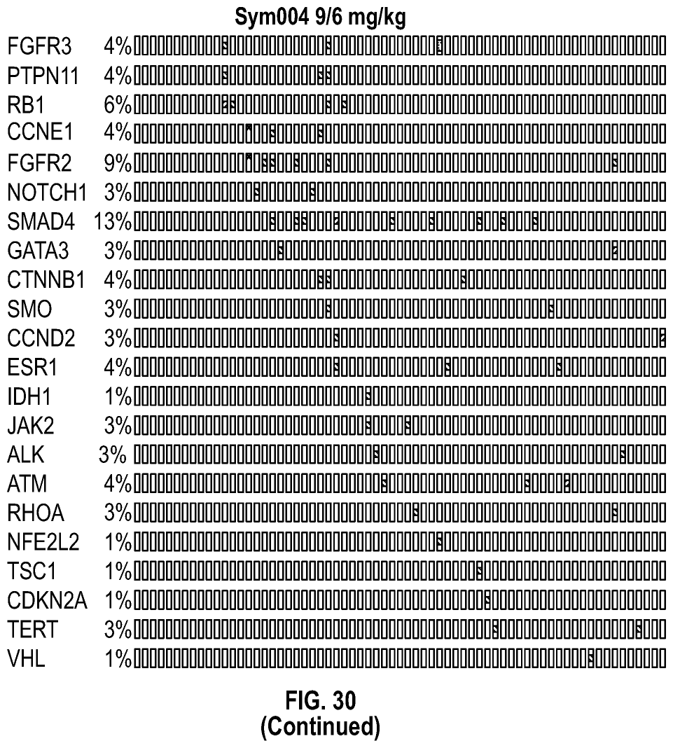

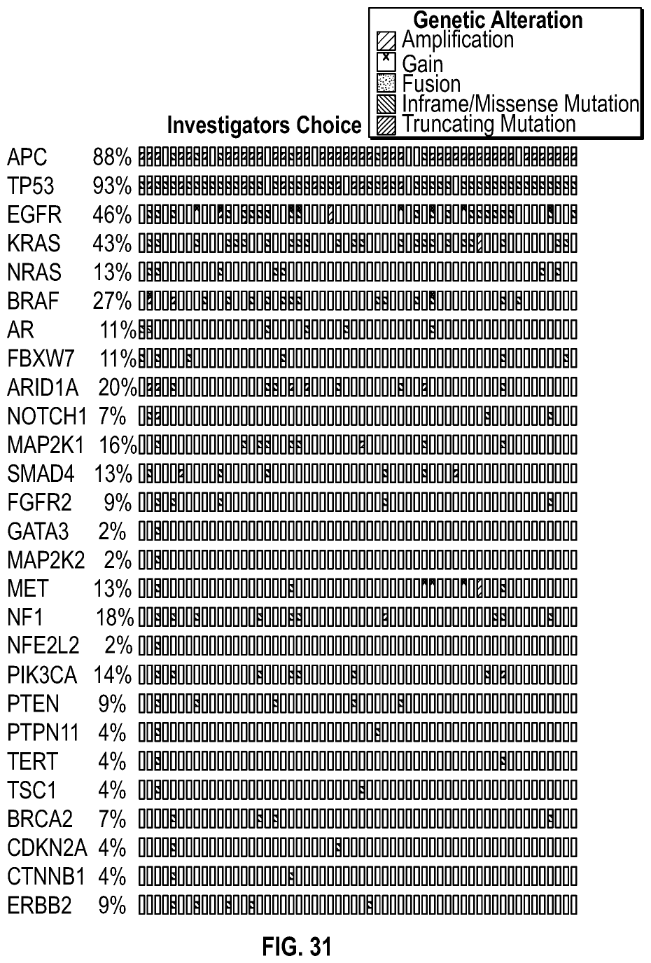

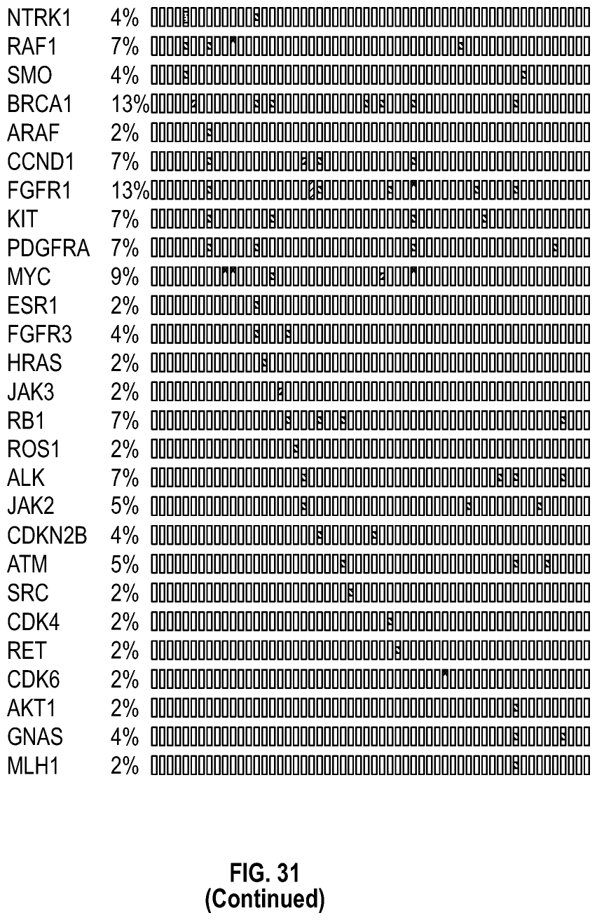

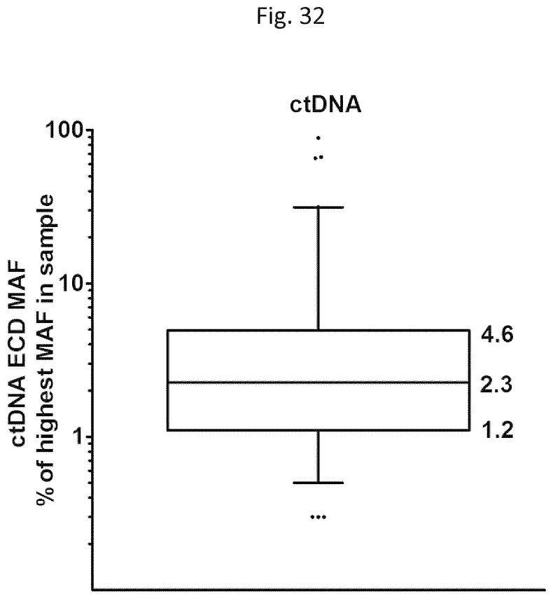

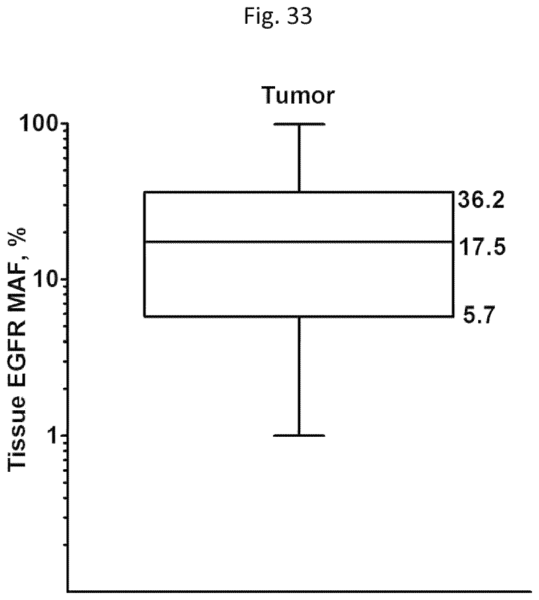

We sought to analyze the dynamics of EGFR ECD mutations in serum from patients during Sym004 treatment. While serum samples are not ideally suited for ctDNA analyses, we found that a digital droplet Polymerase Chain Reaction (PCR) assay detected EGFR ECD mutations in serum. The percentage of EGFR ECD mutations decreased in the majority of patients treated with Sym004, suggesting that subclones carrying EGFR ECD mutations might be targeted by the modotuximab component of Sym004, as shown in the preclinical in vitro and in vivo studies (FIGS. 13 and 14). However, this retained activity did not translate into a clinically meaningful OS benefit, likely due to other co-occurring resistance mechanisms (FIGS. 16A-16C and 29-31) and subclonality of EGFR ECD mutations, as mentioned above (FIGS. 2, 32, and 33). Interestingly, in the two patients who experienced an increase in percentage of one EGFR ECD mutation in ctDNA during Sym004 therapy, other EGFR ECD mutations that were concomitantly detected in the same sample declined following Sym004 therapy (FIG. 14).

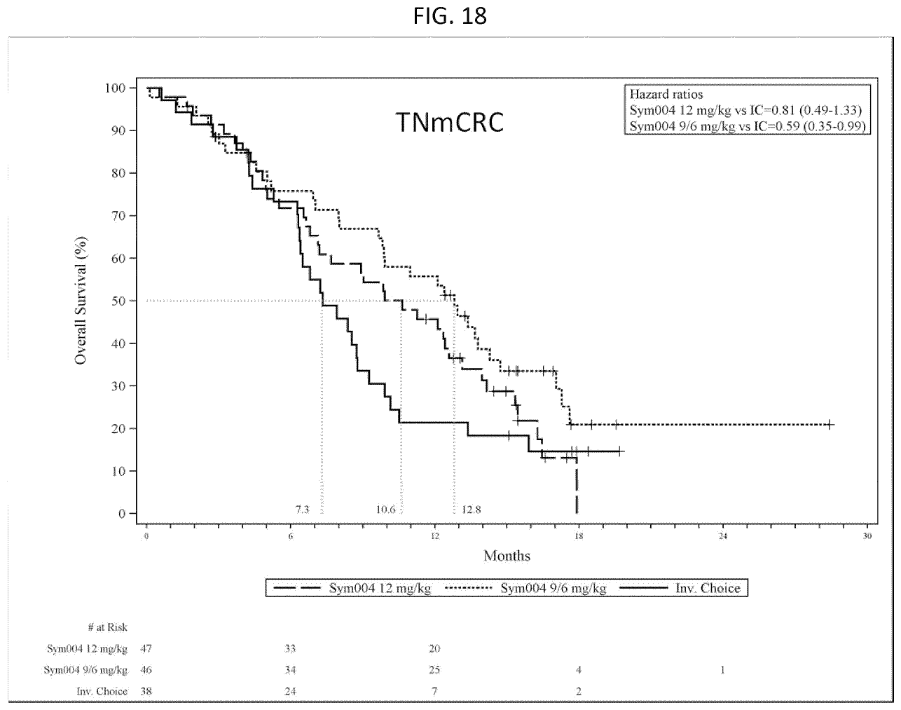

Most notably, we found that the molecular heterogeneity related to resistance markers (which we defined as the number of resistance alterations, including RAS, BRAF, and EGFR ECD mutations and ERBB2 and MET amplifications) was associated with worse OS (FIGS. 16A-16C). This suggests that the occurrence of heterogeneous resistance mutations (including EGFR ECD mutations) may pinpoint a subset of patients in which high genomic complexity due to previous chemotherapy and EGFR blockade impaired effectiveness of Sym004 treatment. To test this hypothesis, we assessed outcomes in patients negative for RAS, BRAF, and EGFR ECD mutations, which we named triple negative mCRC (TNmCRC). Patients in whom the KRAS/NRAS MAF in ctDNA was higher than 20% were excluded from the analysis. We found that the TNmCRC population had a one-year survival rate of 56% (95% CI 40-69) for the Sym004 9/6 treatment group, 46% (95% CI 31-59) for the Sym004 12 group, and 21% (95% CI 9-36) for the IC group. Further, the population had markedly prolonged OS (12.8 months; CI 9.7-14.7) in the 9/6 mg/kg Sym004 treatment arm compared to the IC arm (7.3 months; CI 6.3-8.8) (FIG. 18).

Use of next generation sequencing (NGS) and ctDNA technology will allow selection of patients who will maximally benefit from Sym004 treatment. Indeed, evaluation of OS in TNmCRC patients showed increases in median OS in both Sym004 treatment arms. The present studies show that definition of TNmCRC is an effective method to enrich the patient population for responsiveness to Sym004. In the TNmCRC subpopulation, median OS for patients on the Sym004 9/6 arm (12.8 months, 95% CI 9.7-14.7) was longer than that for either the Sym004 12 arm (10.6, 95% CI 6.8-13.1) or the IC arm (7.3 months, 95% CI, 6.3-8.8). The overall safety profile was better for the 9/6 arm than for the 12 arm, and the 9/6 arm had fewer dose reductions and treatment discontinuations during therapy. The 5.5 month prolongation of median OS in the Sym004 9/6 arm compared to the IC arm (HR 0.59) strongly suggests that the present genetic analysis can delineate uniquely sensitive patient populations. In sum, the results of the present study demonstrate that genotyping of ctDNA can define a patient population likely to benefit from treatment with Sym004 in an extremely challenging clinical setting: mCRC that has developed resistance to EGFR blockade.

SEQ ID NO Table

| SEQ ID NO: | Description |
|---|---|
| 1 | 992 VH |
| 2 | 992 VL |
| 3 | 1024 VH |
| 4 | 1024 VL |
| 5 | 992 H-CDR1 |
| 6 | 992 H-CDR2 |
| 7 | 992 H-CDR3 |
| 8 | 992 L-CDR1 |
| 9 | 992 L-CDR2 |
| 10 | 992 L-CDR3 |
| 11 | 1024 H-CDR1 |
| 12 | 1024 H-CDR2 |
| 13 | 1024 H-CDR3 |
| 14 | 1024 L-CDR1 |
| 15 | 1024 L-CDR2 |
| 16 | 1024 L-CDR3 |
| 17 | P1X VL |
| 18 | P1X VH |
| 19 | P2X VL |
| 20 | P2X VH |
| 21 | P3X VL |
| 22 | P3X VH |
| 23 | human IGHG1 |
| 24 | 992 LC |
| 25 | 1024 LC |
| 26 | 992 HC |
| 27 | 1024 HC |
| 28 | human KRAS |
| 29 | human NRAS |
| 30 | human BRAF |
| 31 | human EGFR |
| 32 | (Gly4-Ser)3 |

VH: heavy chain variable domain
VL: light chain variable domain
H-CDR1-3: heavy chain CDR1-3
L-CDR1-3: light chain CDR1-3
HC: heavy chain
LC: light chain

### SEQUENCES

SEQ ID NO: 1
EVQLQQPGSELVRPGASVKLSCKASGYTFTSYWMHWVKQRPGQGLEWIGNIYPGSRSTNY
DEKFKSKATLTVDTSSSTAYMQLSSLTSEDSAVYYCTRNGDYYVSSGDAMDYWGQGTSVT
VSS
SEQ ID NO: 2
DIQMTQTTSSLSASLGDRVTISCRTSQDIGNYLNWYQQKPDGTVKLLIYYTSRLHSGVPS
RFSGSGSGTDFSLTINNVEQEDVATYFCQHYNTVPPTFGGGTKLEIK
SEQ ID NO: 3
QVQLQQPGAELVEPGGSVKLSCKASGYTFTSHWMHWVKQRPGQGLEWIGEINPSSGRNNY
NEKFKSKATLTVDKSSSTAYMQFSSLTSEDSAVYYCVRYYGYDEAMDYWGQGTSVTVSS
SEQ ID NO: 4
DIVMTQAAFSNPVTLGTSASISCRSSKSLLHSNGITYLYWYLQKPGQSPQLLIYQMSNLA
SGVPDRFSSSGSGTDFTLRISRVEAEDVGVYYCAQNLELPYTFGGGTKLEIK
SEQ ID NO: 5
GYTFTSYW
SEQ ID NO: 6
IYPGSRST
SEQ ID NO: 7
TRNGDYYVSSGDAMDY
SEQ ID NO: 8
QDIGNY
SEQ ID NO: 9
YTS
SEQ ID NO: 10
QHYNTVPPT
SEQ ID NO: 11
GYTFTSHW
SEQ ID NO: 12
INPSSGRN
SEQ ID NO: 13
VRYYGYDEAMDY
SEQ ID NO: 14
KSLLHSNGITY
SEQ ID NO: 15
QMS
SEQ ID NO: 16
AQNLELPYT
SEQ ID NO: 17
DIQMTQSPSTLSASVGDRVTITCRASQSISSWWAWYQQKPGKAPKLLIYDASSLESGVPS
RFSGSGSGTEFTLTISSLQPDDFATYYCQQYHAHPTTFGGGTKVEIK
SEQ ID NO: 18:
QVQLVQSGAEVKKPGSSVKVSCKASGGTFSSYAISWVRQAPGQGLEWMGSIIPIFGTVNY
AQKFQGRVTITADESTSTAYMELSSLRSEDTAVYYCARDPSVNLYWYFDLWGRGTLVTVS
S
SEQ ID NO: 19:
DIVMTQSPDSLAVSLGERATINCKSSQSVLYSPNNKNYLAWYQQKPGQPPKLLIYWASTR
ESGVPDRFSGSGSGTDFTLTISSLQAEDVAVYYCQQYYGSPITFGGGTKVEIK
SEQ ID NO: 20:
QVQLVQSGAEVKKPGSSVKVSCKASGGTFGSYAISWVRQAPGQGLEWMGSIIPIFGAANP
AQKSQGRVTITADESTSTAYMELSSLRSEDTAVYYCAKMGRGKVAFDIWGQGTMVTVSS
SEQ ID NO: 21:
EIVMTQSPATLSVSPGERATLSCRASQSVSSNLAWYQQKPGQAPRLLIYGASTRATGIPA
RFSGSGSGTEFTLTISSLQSEDFAVYYCQDYRTWPRRVFGGGTKVEIK
SEQ ID NO: 22:
QVQLVQSGAEVKKPGASVKVSCKASGYAFTSYGINWVRQAPGQGLEWMGWISAYNGNTYY
AQKLRGRVTMTTDTSTSTAYMELRSLRSDDTAVYYCARDLGGYGSGSVPFDPWGQGTLVT
VSS
SEQ ID NO: 23
SASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKRVEPKSCDKTHTCPPCPAPELLG
GPSVFLFPPKPKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREPQVYTLPPSRE
LEMTKNQVSTCLVKGFYPSDIAVEWESNGQPENNYKTTPPVLDSDGSFFLYSKLTVDKSR
WQQGNVFSCSVMHEALHNHYTQKSLSLSPGK
SEQ ID NO: 24
DIQMTQTTSSLSASLGDRVTISCRTSQDIGNYLNWYQQKPDGTVKLLIYYTSRLHSGVPS
RFSGSGSGTDFSLTINNVEQEDVATYFCQHYNTVPPTFGGGTKLEIKRTVAAPSVFIFPP
SDEQLKSGTASVVCLLNNFYPREAKVQWKVDNALQSGNSQESVTEQDSKDSTYSLSSTLT
LSKADYEKHKVYACEVTHQGLSSPVTKSFNRGEC
SEQ ID NO: 25
DIVMTQAAFSNPVTLGTSASISCRSSKSLLHSNGITYLYWYLQKPGQSPQLLIYQMSNLA
SGVPDRFSSSGSGTDFTLRISRVEAEDVGVYYCAQNLELPYTFGGGTKLEIKRTVAAPSV
FIFPPSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
SEQ ID NO: 26
EVQLQQPGSELVRPGASVKLSCKASGYTFTSYWMHWVKQRPGQGLEWIGNIYPGSRSTNY
DEKFKSKATLTVDTSSSTAYMQLSSLTSEDSAVYYCTRNGDYYVSSGDAMDYWGQGTSVT
VSSASTKGPSVFPLAPSSKSTSGGTAALGCLVKDYFPEPVTVSWNSGALTSGVHTFPAVL
QSSGLYSLSSVVTVPSSSLGTQTYICNVNHKPSNTKVDKRVEPKSCDKTHTCPPCPAPEL
LGGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPS
REEMTKNQVSLTCLVKGFYPSDIAVEWESNGQPENNYKTTPPVLDSDGSFFLYSKLTVDK
SRWQQGNVFSCSVMHEALHNHYTQKSLSLSPG
SEQ ID NO: 27
QVQLQQPGAELVEPGGSVKLSCKASGYTFTSHWMHWVKQRPGQGLEWIGEINPSSGRNNY
NEKFKSKATLTVDKSSSTAYMQFSSLTSEDSAVYYCVRYYGYDEAMDYWGQGTSVTVSSA
STKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKRVEPKSCDKTHTCPPCPAPELLGGP
SVFLFPPKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPPSREEM
TKNQVSLTCLVKGFYPSDIAVEWESNGQPENNYKTTPPVLDSDGSFFLYSKLTVDKSRWQ
QGNVFSCSVMHEALHNHYTQKSLSLSPG
SEQ ID NO: 28
MTEYKLVVVGAGGVGKSALTIQLIQNHFVDEYDPTIEDSYRKQVVIDGETCLLDILDTAG
QEEYSAMRDQYMRTGEGFLCVFAINNTKSFEDIHHYREQIKRVKDSEDVPMVLVGNKCDL
PSRTVDTKQAQDLARSYGIPFIETSAKTRQRVEDAFYTLVREIRQYRLKKISKEEKTPGC
VKIKKCIIM
SEQ ID NO: 29
MTEYKLVVVGAGGVGKSALTIQLIQNHFVDEYDPTIEDSYRKQVVIDGETCLLDILDTAG
QEEYSAMRDQYMRTGEGFLCVFAINNSKSFADINLYREQIKRVKDSDDVPMVLVGNKCDL
PTRTVDTKQAHELAKSYGIPFIETSAKTRQGVEDAFYTLVREIRQYRMKKLNSSDDGTQG
CMGLPCVVM
SEQ ID NO: 30
MAALSGGGGGGAEPGQALFNGDMEPEAGAGAGAAASSAADPAIPEEVWNIKQMIKLTQEH
IEALLDKFGGEHNPPSIYLEAYEEYTSKLDALQQREQQLLESLGNGTDFSVSSSASMDTV
TSSSSSSLSVLPSSLSVFQNPTDVARSNPKSPQKPIVRVFLPNKQRTVVPARCGVTVRDS
LKKALMMRGLIPECCAVYRIQDGEKKPIGWDTDISWLTGEELHVEVLENVPLTTHNFVRK
TFFTLAFCDFCRKLLFQGFRCQTCGYKFHQRCSTEVPLMCVNYDQLDLLFVSKFFEHHPI
PQEEASLAETALTSGSSPSAPASDSIGPQILTSPSPSKSIPIPQPFRPADEDHRNQFGQR
DRSSSAPNVHINTIEPVNIDDLIRDQGFRGDGGSTTGLSATPPASLPGSLTNVKALQKSP
GPQRERKSSSSSEDRNRMKTLGRRDSSDDWEIPDGQITVGQRIGSGSFGTVYKGKWHGDV
AVKMLNVTAPTPQQLQAFKNEVGVLRKTRHVNILLFMGYSTKPQLAIVTQWCEGSSLYHH
LHIIETKFEMIKLIDIARQTAQGMDYLHAKSIIHRDLKSNNIFLHEDLTVKIGDFGLATV
KSRWSGSHQFEQLSGSILWMAPEVIRMQDKNPYSFQSDVYAFGIVLYELMTGQLPYSNIN
NRDQIIFMVGRGYLSPDLSKVRSNCPKAMKRLMAECLKKKRDERPLFPQILASIELLARS
LPKIHRSASEPSLNRAGFQTEDFSLYACASPKTPIQAGGYGAFPVH
SEQ ID NO: 31
MRPSGTAGAALLALLAALCPASRALEEKKVCQGTSNKLTQLGTFEDHFLSLQRMFNNCEV
VLGNLEITYVQRNYDLSFLKTIQEVAGYVLIALNTVERIPLENLQIIRGNMYYENSYALA
VLSNYDANKTGLKELPMRNLQEILHGAVRFSNNPALCNVESIQWRDIVSSDFLSNMSMDF
QNHLGSCQKCDPSCPNGSCWGAGEENCQKLTKIICAQQCSGRCRGKSPSDCCHNQCAAGC
TGPRESDCLVCRKFRDEATCKDTCPPLMLYNPTTYQMDVNPEGKYSFGATCVKKCPRNYV
VTDHGSCVRACGADSYEMEEDGVRKCKKCEGPCRKVCNGIGIGEFKDSLSINATNIKHFK
NCTSISGDLHILPVAFRGDSFTHTPPLDPQELDILKTVKEITGFLLIQAWPENRTDLHAF
ENLEIIRGRTKQHGQFSLAVVSLNITSLGLRSLKEISDGDVIISGNKNLCYANTINWKKL
FGTSGQKTKIISNRGENSCKATGQVCHALCSPEGCWGPEPRDCVSCRNVSRGRECVDKCN
LLEGEPREFVENSECIQCHPECLPQAMNITCTGRGPDNCIQCAHYIDGPHCVKTCPAGVM
GENNTLVWKYADAGHVCHLCHPNCTYGCTGPGLEGCPTNGPKIPSIATGMVGALLLLLVV
ALGIGLFMRRRHIVRKRTLRRLLQERELVEPLTPSGEAPNQALLRILKETEFKKIKVLGS
GAFGTVYKGLWIPEGEKVKIPVAIKELREATSPKANKEILDEAYVMASVDNPHVCRLLGI
CLTSTVQLITQLMPFGCLLDYVREHKDNIGSQYLLNWCVQIAKGMNYLEDRRLVHRDLAA
RNVLVKTPQHVKITDFGLAKLLGAEEKEYHAEGGKVPIKWMALESILHRIYTHQSDVWSY
GVTVWELMTFGSKPYDGIPASEISSILEKGERLPQPPICTIDVYMIMVKCWMIDADSRPK
FRELIIEFSKMARDPQRYLVIQGDERMHLPSPTDSNFYRALMDEEDMDDVVDADEYLIPQ
QGFFSSPSTSRTPLLSSLSATSNNSTVACIDRNGLQSCPIKEDSFLQRYSSDPTGALTED
SIDDTFLPVPEYINQSVPKRPAGSVQNPVYHNQPLNPAPSRDPHYQDPHSTAVGNPEYLN
TVQPTCVNSTFDSPAHWAQKGSHQISLDNPDYQQDFFPKEAKPNGIFKGSTAENAEYLRV
APQSSEFIGA
SEQ ID NO: 32
GGGGSGGGGSGGGGS

1. A method for treating cancer in a patient, comprising:
a) selecting a patient with said cancer from whom a tumor DNA sample:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146);
ii) has a MAF of less than 0.1% for BRAF mutation V600E; and
iii) has a MAF of less than 0.1% for EGFR ECD mutations V441 D, V441G, S464L, G465E, G465R, and S492R, and
b) administering to the patient an anti-EGFR antibody composition comprising two anti-human EGFR antibodies that bind to distinct epitopes in the EGFR extracellular domain (ECD).

2. A method for treating cancer in a patient, comprising:
a) selecting a patient with said cancer from whom a tumor DNA sample:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146); and
ii) has a MAF of less than 0.1% for BRAF mutation V600E, and
b) administering to the patient an anti-EGFR antibody composition comprising two anti-human EGFR antibodies that bind to distinct epitopes in the EGFR extracellular domain (ECD).

3. The method of claim 1, wherein the tumor DNA sample has no detectable levels of EGFR ECD mutations V441D, V441G, S464L, G465E, G465R, and S492R.

4. The method of any one of claims 1-3, wherein the tumor DNA sample has no detectable levels of BRAF mutation V600E.

5. The method of any one of claims 1-4, wherein the tumor DNA sample has been determined to be also negative for gene amplification of MET and ERBB2.

6. The method of claim 5, wherein the tumor DNA sample has been determined to be also negative for gene amplification of KRAS.

7. The method of any one of claims 1-6, wherein the cancer is selected from the group consisting of colorectal cancer, non-small cell lung cancer (NSCLC), and squamous cell carcinoma of the head and neck (SCCHN).

8. The method of claim 7, wherein the cancer is colorectal cancer.

9. The method of claim 8, wherein the cancer is metastatic colorectal cancer.

10. The method of any one of claims 1-9, wherein the patient has received prior treatment with an anti-EGFR antibody that is not an antibody in said antibody composition.

11. The method of claim 10, wherein the prior anti-EGFR antibody is selected from the group consisting of cetuximab, panitumumab, zalutumumab, nimotuzumab, ICR62, mAb806, matuzumab, and antibodies capable of binding the same epitope as any of these.

12. The method of claim 10, wherein the patient has been treated with cetuximab, panitumumab, or both.

13. The method of any one of claims 1-12, wherein said cancer is resistant or partially resistant to prior treatment with an anti-EGFR antibody that is not an antibody in said antibody composition.

14. The method of claim 13, wherein the prior anti-EGFR antibody is selected from the group consisting of cetuximab, panitumumab, zalutumumab, nimotuzumab, ICR62, mAb806, matuzumab, and antibodies capable of binding the same epitope as any of these.

15. The method of claim 13, wherein said cancer is resistant or partially resistant to prior treatment with cetuximab, panitumumab, or both.

16. The method of any one of claims 13-15, wherein the resistance or partial resistance has been determined by assaying a sample of cancer cells isolated from said patient.

17. The method of any one of claims 1-16, wherein the patient has demonstrated intolerance to, or failed on prior treatment with, at least one chemotherapy agent selected from the group consisting of 5-FU, oxaliplatin, irinotecan, FOLFOX (folinic acid, fluorouracil and oxaliplatin), and FOLFIRI (folinic acid, fluorouracil and irinotecan).

18. The method of any one of claims 1-17, wherein the tumor DNA sample is a circulating tumor (ct) DNA sample from the patient.

19. The method of any one of claims 1-17, wherein the tumor DNA sample is obtained from a tumor tissue sample or circulating tumor cells from the patient.

20. The method of any one of claims 1-19, wherein the anti-EGFR antibody composition has at least one of the following properties:
a) enhances internalization and degradation of EGFR;
b) induces complement-dependent cytotoxicity (CDC);
c) induces differentiation of tumor cells in vivo; and
d) increases involucrin expression in vivo.

21. The method of claim 20, wherein the anti-EGFR antibody composition has all of said properties.

22. The method of any one of claims 1-21, wherein the anti-EGFR antibody composition comprises a first anti-human EGFR antibody and a second anti-human EGFR antibody, wherein:
the first anti-human EGFR antibody comprises the heavy chain CDR1, CDR2, and CDR3 in SEQ ID NO: 1 and the light chain CDR1, CDR2, and CDR3 in SEQ ID NO: 2; and
the second anti-human EGFR antibody comprises the heavy chain CDR1, CDR2, and CDR3 in SEQ ID NO: 3 and the light chain CDR1, CDR2, and CDR3 in SEQ ID NO: 4.

23. The method of claim 22, wherein
the first anti-human EGFR antibody comprises a heavy chain comprising the amino acid sequence of SEQ ID NO: 1 and a light chain comprising the amino acid sequence of SEQ ID NO: 2; and
the second anti-human EGFR antibody comprises a heavy chain comprising the amino acid sequence of SEQ ID NO: 3 and a light chain comprising the amino acid sequence of SEQ ID NO: 4.

24. The method of claim 23, wherein
the first anti-human EGFR antibody comprises a heavy chain comprising the amino acid sequence of SEQ ID NO: 26 and a light chain comprising the amino acid sequence of SEQ ID NO: 24; and
the second anti-human EGFR antibody comprises a heavy chain comprising the amino acid sequence of SEQ ID NO: 27 and a light chain comprising the amino acid sequence of SEQ ID NO: 25.

25. The method of any one of claims 1-23, wherein the first and second anti-human EGFR antibodies of the composition are of isotype IgG1 or IgG2.

26. The method of any one of claims 1-25, wherein the ratio of the first anti-human EGFR antibody relative to the second anti-human EGFR antibody is 1.1.

27. The method of any one of claims 1-26, wherein the antibody composition is administered to the patient at a loading dose of 9 mg/kg, followed by a weekly dose of 6 mg/kg.

28. The method of any one of claims 1-26, wherein the antibody composition is administered to the patient at a weekly dose of 12 mg/kg.

29. The method of any one of claims 1-28, wherein the patient is human.

30. A method for treating cancer in a human patient, comprising administering to the patient an anti-EGFR antibody composition comprising:
a first anti-human EGFR antibody that comprises a heavy chain comprising the amino acid sequence of SEQ ID NO: 26 and a light chain comprising the amino acid sequence of SEQ ID NO: 24; and
a second anti-human EGFR antibody that comprises a heavy chain comprising the amino acid sequence of SEQ ID NO: 27 and a light chain comprising the amino acid sequence of SEQ ID NO: 25;
wherein the antibody composition is administered intravenously to the patient at a loading dose of 9 mg/kg, followed one week later by a weekly dose of 6 mg/kg.

31. A method for treating cancer in a human patient, comprising:
a) selecting a patient with said cancer from whom a tumor DNA sample:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146),
ii) has a MAF of less than 0.1% for BRAF mutation V600E, and
iii) has a MAF of less than 0.1% for EGFR ECD mutations V441D, V441G, S464L, G465E, G465R, and S492R; and
b) administering to the patient an anti-EGFR antibody composition comprising:
a first anti-human EGFR antibody that comprises a heavy chain comprising the amino acid sequence of SEQ ID NO: 26 and a light chain comprising the amino acid sequence of SEQ ID NO: 24; and
a second anti-human EGFR antibody that comprises a heavy chain comprising the amino acid sequence of SEQ ID NO: 27 and a light chain comprising the amino acid sequence of SEQ ID NO: 25;
wherein the antibody composition is administered intravenously to the patient at a loading dose of 9 mg/kg, followed one week later by a weekly dose of 6 mg/kg.

32. A method for treating cancer in a patient, comprising:
a) selecting a patient with said cancer from whom a tumor DNA sample:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146), and
ii) has a MAF of less than 0.1% for of BRAF mutation V600E; and
b) administering to the patient an anti-EGFR antibody composition comprising:
a first anti-human EGFR antibody that comprises a heavy chain comprising the amino acid sequence of SEQ ID NO: 26 and a light chain comprising the amino acid sequence of SEQ ID NO: 24; and
a second anti-human EGFR antibody that comprises a heavy chain comprising the amino acid sequence of SEQ ID NO: 27 and a light chain comprising the amino acid sequence of SEQ ID NO: 25;
wherein the antibody composition is administered intravenously to the patient at a loading dose of 9 mg/kg, followed one week later by a weekly dose of 6 mg/kg.

33. The method of claim 31, wherein the tumor DNA sample has no detectable levels of EGFR ECD mutations V441D, V441G, S464L, G465E, G465R, and S492R.

34. The method of any one of claims 31-33, wherein the tumor DNA sample has no detectable levels of BRAF mutation V600E.

35. The method of any one of claims 30-34, wherein the patient has metastatic colorectal cancer.

36. Use of an antibody composition comprising two anti-human EGFR antibodies that bind to distinct epitopes in the EGFR extracellular domain (ECD) for the manufacture of a medicament for treating cancer in a patient, wherein a tumor DNA sample from the patient:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146);
ii) has a MAF of less than 0.1% for BRAF mutation V600E; and
iii) has a MAF of less than 0.1% for EGFR ECD mutations V441D, V441G, S464L, G465E, G465R, and S492R.

37. Use of an antibody composition comprising two anti-human EGFR antibodies that bind to distinct epitopes in the EGFR extracellular domain (ECD) for the manufacture of a medicament for treating cancer in a patient, wherein a tumor DNA sample from the patient:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146); and
ii) has a MAF of less than 0.1% for BRAF mutation V600E.

38. The use of claim 36 or 37, wherein the medicament is for treating cancer in a patient in the method of any one of claims 1-35.

39. An antibody composition comprising two anti-human EGFR antibodies that bind to distinct epitopes in the EGFR extracellular domain (ECD) for use in treating cancer in a patient in a method comprising:
a) selecting a patient with said cancer from whom a tumor DNA sample:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146);
ii) has a MAF of less than 0.1% for BRAF mutation V600E; and
iii) has a MAF of less than 0.1% for EGFR ECD mutations V441 D, V441G, S464L, G465E, G465R, and S492R, and
b) administering to the patient an anti-EGFR antibody composition comprising two anti-human EGFR antibodies that bind to distinct epitopes in the EGFR extracellular domain (ECD).

40. An antibody composition comprising two anti-human EGFR antibodies that bind to distinct epitopes in the EGFR extracellular domain (ECD) for use in treating cancer in a patient in a method comprising:
a) selecting a patient with said cancer from whom a tumor DNA sample:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146); and
ii) has a MAF of less than 0.1% for BRAF mutation V600E, and
b) administering to the patient an anti-EGFR antibody composition comprising two anti-human EGFR antibodies that bind to distinct epitopes in the EGFR extracellular domain (ECD).

41. The antibody composition of claim 39 or 40, for use in treating cancer in a patient in the method of any one of claims 1-35.

42. An article of manufacture suitable for treating cancer in a patient, comprising an antibody composition comprising two anti-human EGFR antibodies that bind to distinct epitopes in the EGFR extracellular domain (ECD), wherein said treatment comprises:
a) selecting a patient with said cancer from whom a tumor DNA sample:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146);
ii) has a MAF of less than 0.1% for BRAF mutation V600E; and
iii) has a MAF of less than 0.1% for EGFR ECD mutations V441 D, V441G, S464L, G465E, G465R, and S492R, and
b) administering to the patient the antibody composition.

43. An article of manufacture suitable for treating cancer in a patient, comprising an antibody composition comprising two anti-human EGFR antibodies that bind to distinct epitopes in the EGFR extracellular domain (ECD), wherein said treatment comprises:
a) selecting a patient with said cancer from whom a tumor DNA sample:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146); and
ii) has a MAF of less than 0.1% for BRAF mutation V600E, and
b) administering to the patient the antibody composition.

44. The article of manufacture of claim 42 or 43, wherein the article is suitable for treating cancer in a patient in the method of any one of claims 1-35.

45. A kit suitable for treating cancer in a patient from whom a tumor DNA sample:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146);
ii) has a MAF of less than 0.1% for BRAF mutation V600E; and
iii) has a MAF of less than 0.1% for EGFR ECD mutations V441D, V441G, S464L, G465E, G465R, and S492R,
wherein the kit comprises an antibody composition comprising two anti-human EGFR antibodies that bind to distinct epitopes in the EGFR extracellular domain (ECD).

46. A kit suitable for treating cancer in a patient from whom a tumor DNA sample:
i) has a mutant allele frequency (MAF) of less than 20% for (1) mutations in KRAS exon 2 (codons 12 and 13), exon 3 (codons 59 and 61), and exon 4 (codons 117 and 146); and (2) mutations in NRAS exon 2 (codons 12 and 13), exon 3 (codons 59 and 61), and exon 4 (codons 117 and 146); and
ii) has a MAF of less than 0.1% for BRAF mutation V600E;
wherein the kit comprises an antibody composition comprising two anti-human EGFR antibodies that bind to distinct epitopes in the EGFR extracellular domain (ECD).

47. The kit of claim 45 or 46, wherein the kit is suitable for treating cancer in a patient in the method of any one of claims 1-35.

48. The use of claim 36 or 38, the antibody composition of claim 39 or 41, the article of manufacture of claim 42 or 44, or the kit of claim 45 or 47, wherein the tumor DNA sample:
a) has no detectable levels of EGFR ECD mutations V441D, V441G, S464L, G465E, G465R, and S492R;
b) has no detectable levels of BRAF mutation V600E; or
c) both a) and b).

49. The use of any one of claims 36-38, the antibody composition of any one of claims 39-41, the article of manufacture of any one of claims 42-44, or the kit of any one of claims 45-47, wherein the tumor DNA sample has no detectable levels of BRAF mutation V600E.

50. The use of any one of claims 36-38, the antibody composition of any one of claims 39-41, the article of manufacture of any one of claims 42-44, or the kit of any one of claims 45-47, wherein the anti-EGFR antibody composition comprises a first anti-human EGFR antibody and a second anti-human EGFR antibody, wherein:
the first anti-human EGFR antibody comprises the heavy chain CDR1, CDR2, and CDR3 in SEQ ID NO: 1 and the light chain CDR1, CDR2, and CDR3 in SEQ ID NO: 2; and
the second anti-human EGFR antibody comprises the heavy chain CDR1, CDR2, and CDR3 in SEQ ID NO: 3 and the light chain CDR1, CDR2, and CDR3 in SEQ ID NO: 4.

51. The use of any one of claims 36-38, the antibody composition of any one of claims 39-41, the article of manufacture of any one of claims 42-44, or the kit of any one of claims 45-47, wherein the anti-EGFR antibody composition comprises a first anti-human EGFR antibody and a second anti-human EGFR antibody, wherein:
the first anti-human EGFR antibody comprises a heavy chain comprising the amino acid sequence of SEQ ID NO: 1 and a light chain comprising the amino acid sequence of SEQ ID NO: 2; and
the second anti-human EGFR antibody comprises a heavy chain comprising the amino acid sequence of SEQ ID NO: 3 and a light chain comprising the amino acid sequence of SEQ ID NO: 4.

52. The use of any one of claims 36-38, the antibody composition of any one of claims 39-41, the article of manufacture of any one of claims 42-44, or the kit of any one of claims 45-47, wherein the anti-EGFR antibody composition comprises a first anti-human EGFR antibody and a second anti-human EGFR antibody, wherein:
the first anti-human EGFR antibody comprises a heavy chain comprising the amino acid sequence of SEQ ID NO: 26 and a light chain comprising the amino acid sequence of SEQ ID NO: 24; and
the second anti-human EGFR antibody comprises a heavy chain comprising the amino acid sequence of SEQ ID NO: 27 and a light chain comprising the amino acid sequence of SEQ ID NO: 25.


SEQUENCE LISTING (32 sequences)

SEQ ID NO: 1 - protein, 123 aa, Artificial Sequence
  source: /note=“Description of Artificial Sequence Synthetic polypeptide”
EVQLQQPGSELVRPGASVKLSCKASGYTFTSYWMHWVKQRPGQGLEWIGNIYPGSRSTNYDEKFKSKATLTVDTSSSTAYMQLSSLTSEDSAVYYCTRNGDYYVSSGDAMDYWGQGTSVTVSS

SEQ ID NO: 2 - protein, 107 aa, Artificial Sequence
  source: /note=“Description of Artificial Sequence Synthetic polypeptide”
DIQMTQTTSSLSASLGDRVTISCRTSQDIGNYLNWYQQKPDGTVKLLIYYTSRLHSGVPSRFSGSGSGTDFSLTINNVEQEDVATYFCQHYNTVPPTFGGGTKLEIK

SEQ ID NO: 3 - protein, 119 aa, Artificial Sequence
  source: /note=“Description of Artificial Sequence Synthetic polypeptide”
QVQLQQPGAELVEPGGSVKLSCKASGYTFTSHWMHWVKQRPGQGLEWIGEINPSSGRNNYNEKFKSKATLTVDKSSSTAYMQFSSLTSEDSAVYYCVRYYGYDEAMDYWGQGTSVTVSS

SEQ ID NO: 4 - protein, 112 aa, Artificial Sequence
  source: /note=“Description of Artificial Sequence Synthetic polypeptide”
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPYTFGGGTKLEIK

SEQ ID NO: 5 - protein, 8 aa, Artificial Sequence
  source: /note=“Description of Artificial Sequence Synthetic peptide”
GYTFTSYW

SEQ ID NO: 6 - protein, 8 aa, Artificial Sequence
  source: /note=“Description of Artificial Sequence Synthetic peptide”
IYPGSRST

SEQ ID NO: 7 - protein, 16 aa, Artificial Sequence
  source: /note=“Description of Artificial Sequence Synthetic peptide”
TRNGDYYVSSGDAMDY

SEQ ID NO: 8 - protein, 6 aa, Artificial Sequence
  source: /note=“Description of Artificial Sequence Synthetic peptide”
QDIGNY

SEQ ID NO: 9 - protein, 3 aa, Artificial Sequence
  source: /note=“Description of Artificial Sequence Synthetic peptide”
YTS

SEQ ID NO: 10 - protein, 9 aa, Artificial Sequence
  source: /note=“Description of Artificial Sequence Synthetic peptide”
QHYNTVPPT

SEQ ID NO: 11 - protein, 8 aa, Artificial Sequence
  source: /note=“Description of Artificial Sequence Synthetic peptide”
GYTFTSHW

SEQ ID NO: 12 - protein, 8 aa, Artificial Sequence
  source: /note=“Description of Artificial Sequence Synthetic peptide”
INPSSGRN

SEQ ID NO: 13 - protein, 12 aa, Artificial Sequence
  source: /note=“Description of Artificial Sequence Synthetic peptide”
VRYYGYDEAMDY

SEQ ID NO: 14 - protein, 11 aa, Artificial Sequence
  source: /note=“Description of Artificial Sequence Synthetic peptide”
KSLLHSNGITY

SEQ ID NO: 15 - protein, 3 aa, Artificial Sequence
  source: /note=“Description of Artificial Sequence Synthetic peptide”
QMS

SEQ ID NO: 16 - protein, 9 aa, Artificial Sequence
  source: /note=“Description of Artificial Sequence Synthetic peptide”
AQNLELPYT

SEQ ID NO: 17 - protein, 107 aa, Artificial Sequence
  source: /note=“Description of Artificial Sequence Synthetic polypeptide”
DIQMTQSPSTLSASVGDRVTITCRASQSISSWWAWYQQKPGKAPKLLIYDASSLESGVPSRFSGSGSGTEFTLTISSLQPDDFATYYCQQYHAHPTTFGGGTKVEIK

SEQ ID NO: 18 - protein, 121 aa, Artificial Sequence
  source: /note=“Description of Artificial Sequence Synthetic polypeptide”
QVQLVQSGAEVKKPGSSVKVSCKASGGTFSSYAISWVRQAPGQGLEWMGSIIPIFGTVNYAQKFQGRVTITADESTSTAYMELSSLRSEDTAVYYCARDPSVNLYWYFDLWGRGTLVTVSS

SEQ ID NO: 19 - protein, 113 aa, Artificial Sequence
  source: /note=“Description of Artificial Sequence Synthetic polypeptide”
DIVMTQSPDSLAVSLGERATINCKSSQSVLYSPNNKNYLAWYQQKPGQPPKLLIYWASTRESGVPDRFSGSGSGTDFTLTISSLQAEDVAVYYCQQYYGSPITFGGGTKVEIK

SEQ ID NO: 20 - protein, 119 aa, Artificial Sequence
  source: /note=“Description of Artificial Sequence Synthetic polypeptide”
QVQLVQSGAEVKKPGSSVKVSCKASGGTFGSYAISWVRQAPGQGLEWMGSIIPIFGAANPAQKSQGRVTITADESTSTAYMELSSLRSEDTAVYYCAKMGRGKVAFDIWGQGTMVTVSS

SEQ ID NO: 21 - protein, 108 aa, Artificial Sequence
  source: /note=“Description of Artificial Sequence Synthetic polypeptide”
EIVMTQSPATLSVSPGERATLSCRASQSVSSNLAWYQQKPGQAPRLLIYGASTRATGIPARFSGSGSGTEFTLTISSLQSEDFAVYYCQDYRTWPRRVFGGGTKVEIK

SEQ ID NO: 22 - protein, 123 aa, Artificial Sequence
  source: /note=“Description of Artificial Sequence Synthetic polypeptide”
QVQLVQSGAEVKKPGASVKVSCKASGYAFTSYGINWVRQAPGQGLEWMGWISAYNGNTYYAQKLRGRVTMTTDTSTSTAYMELRSLRSDDTAVYYCARDLGGYGSGSVPFDPWGQGTLVTVSS

SEQ ID NO: 23 - protein, 331 aa, Homo sapiens
SASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 214 aa, Artificial Sequence
  source: /note=“Description of Artificial Sequence Synthetic polypeptide”
DIQMTQTTSSLSASLGDRVTISCRTSQDIGNYLNWYQQKPDGTVKLLIYYTSRLHSGVPSRFSGSGSGTDFSLTINNVEQEDVATYFCQHYNTVPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 219 aa, Artificial Sequence
  source: /note=“Description of Artificial Sequence Synthetic polypeptide”
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 452 aa, Artificial Sequence
  source: /note=“Description of Artificial Sequence Synthetic polypeptide”
EVQLQQPGSELVRPGASVKLSCKASGYTFTSYWMHWVKQRPGQGLEWIGNIYPGSRSTNYDEKFKSKATLTVDTSSSTAYMQLSSLTSEDSAVYYCTRNGDYYVSSGDAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - protein, 448 aa, Artificial Sequence
  source: /note=“Description of Artificial Sequence Synthetic polypeptide”
QVQLQQPGAELVEPGGSVKLSCKASGYTFTSHWMHWVKQRPGQGLEWIGEINPSSGRNNYNEKFKSKATLTVDKSSSTAYMQFSSLTSEDSAVYYCVRYYGYDEAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 28 - protein, 189 aa, Homo sapiens [sequence omitted]

SEQ ID NO: 29 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 30 - protein, 766 aa, Homo sapiens [sequence omitted]

SEQ ID NO: 31 - protein, 1210 aa, Homo sapiens [sequence omitted]

SEQ ID NO: 32 - protein, 15 aa, Artificial Sequence
  source: /note=“Description of Artificial Sequence Synthetic peptide”
GGGGSGGGGSGGGGS